(19) United States
(12) Patent Application Publication
(10) Pub. No.: US 2026/0345830 A1
(43) Pub. Date: Nov. 5, 2026

(54) AAV VECTORS WITH MYELIN PROTEIN ZERO PROMOTER AND USES THEREOF FOR TREATING SCHWANN CELL-ASSOCIATED DISEASES LIKE CHARCOT-MARIE-TOOTH DISEASE

(71) Applicant: THE CYPRUS FOUNDATION FOR MUSCULAR DYSTROPHY RESEARCH, Nicosia (CY)

(72) Inventors: Kleopas KLEOPA, Nicosia (CY); Alexia KAGIAVA, Nicosia (CY); Natasa SCHIZA, Nicosia (CY); Irene SARGIANNIDOU, Nicosia (CY)

(21) Appl. No.: 19/547,202

(22) Filed: Feb. 23, 2026

Related U.S. Application Data
(63) Continuation of application No. 17/616,006, filed on Jun. 3, 2020, now Pat. No. 12,582,725.

(30) Foreign Application Priority Data
Jun. 3, 2019 (GB) 1907882.3

(51) Int. Cl. A61K 48/00; A61P 25/02; C07K 14/47; C07K 14/705; C12N 15/86

(52) U.S. Cl. CPC A61K 48/0058; A61K 48/0075; A61P 25/02; C07K 14/47; C07K 14/70503; C12N 15/86; C12N 2750/14143; C12N 2830/008

(57) ABSTRACT

The present invention provides viral vectors for use in the treatment and prevention of diseases associated with Schwann cells by delivering polynucleotides specifically to Schwann cells and achieving Schwann cell specific expression. The present invention has particular application in treatment and prevention of Charcot-Marie-Tooth disease and other demyelinating neuropathies.

### REFERENCE TO SEQUENCE LISTING

The sequence listing submitted Jun. 26, 2026, as an xml file named P7347UScon1 Seq List.xml, created on Apr. 4, 2026, and having a size of 95,557 bytes is herein incorporated by reference pursuant to 37 C.F.R. § 1.52(e)(5).

### FIELD OF THE INVENTION

The present invention relates to viral vectors targeting diseases associated with Schwann cells.

### BACKGROUND

Charcot-Marie-Tooth (CMT) disease encompasses numerous types of non-syndromic inherited neuropathies, which together are considered to be one of the most common neurogenetic disorders, with a frequency of affected individuals reaching 1:2500 of the general population (1, 2). CMT neuropathies are characterized by the involvement of an ever-increasing number of causative genes and overlapping phenotypes caused by different genes. Moreover, several different genes may cause identical phenotypes. Despite the increasing understanding of the complex genetic basis and diverse disease mechanisms underpinning CMT neuropathies, there is currently no effective treatment for any of the CMT forms and only symptomatic and supportive therapy can be offered to patients. Thus, there is a great need for new treatment strategies for CMT. In the last two decades there has been an effort to develop gene therapies for the treatment of CMT. While different gene therapy approaches hold promise for the future to treat diseases of the central and peripheral nervous system (PNS), multiple challenges remain to be overcome (3).

For example, (49) shows how it is possible to achieve a therapeutic effect in the treatment of CMT4C using a lentiviral vector. However, this effect was partial and the lentiviral vector has safety limitations for in vivo human therapies. Previously, other vectors such as adeno-associated viral vectors (AAVs) were not considered as useful, as despite being more stable and not integrating into the host genome, due to their maximum packaging capacity of inserts of approximately 4.4 kb in length, the utility of AAVs in gene therapy strategies was limited, especially in cases where the gene to be replaced is relatively long.

Gene therapy techniques targeting Schwann cells can be applied to many other diseases associated with Schwann cells aside from CMT, for example motor neuron disease (MND), and include those not exclusively caused by genetic factors. Many of these diseases have multiple causes and are not well understood, and therefore targeting these diseases using viral vectors may be particularly advantageous.

Overall, there remains a need for improved methods of targeting diseases associated with Schwann cells, including demyelinating neuropathies such as CMT, to achieve better therapeutic effects.

### SUMMARY OF THE INVENTION

The inventors have developed for the first time a useful means of delivering polynucleotides, for example therapeutic polynucleotides to the Schwann cells of the peripheral nervous system (PNS) and driving expression of said polynucleotides specifically in Schwann cells. The present invention can be applied to the treatment of diseases associated with Schwann cells, and is considered to be particularly beneficial when applied to the treatment of demyelinating neuropathies such as Charcot-Marie-Tooth disease (CMT). However, the underlying mechanism of the invention is considered to be applicable to many other diseases that affect Schwann cells and is also considered to have general utility in any situation where delivery of a polynucleotide to a Schwann cell is considered to be advantageous, for example in imaging of Schwann cells.

A feature of one aspect of the present invention is the use of an AAV vector to achieve transcription of a first nucleic acid resulting in the production of first polynucleotide of interest specifically in Schwann cells of the PNS. In some embodiments, this cell-type specific expression is achieved using a myelin specific promoter, and in some embodiments this is achieved using a minimal version of a myelin specific promoter.

Another feature of the present invention is the provision of a minimal myelin specific promoter, which in some embodiments is based on the sequence of the full length myelin protein zero (Mpz) promoter. In some embodiments, viral vectors that comprise a shorter minimal promoter allow larger nucleic acid sequences, for example therapeutic nucleic acid sequences, to be included in the vector and delivered to Schwann cells. This is considered to have the advantageous property of providing a universal vector for delivery of nucleic acids to the Schwann cells, and can be used to treat a large range of diseases, since current approaches are limited in, for example, the genes that can be expressed from the viral vector due to their size.

### DETAILED DESCRIPTION OF THE INVENTION

The invention is as defined by the claims.

The invention generally provides a viral vector as described herein for use in medicine and also a provides a method of therapy that comprises administering a vector according to the invention, for example administering by any of the means described herein.

A first aspect of the invention provides a viral vector for use in treating or preventing a disease associated with Schwann cells. In some embodiments, the viral vector comprises a first nucleic acid sequence that can be transcribed into a first polynucleotide.

The viral vector may be any viral vector.

Viral vectors are well known in the art and examples include but are not limited to: adeno-associated viral vectors (AAV vectors); lentiviral vectors (e.g. those derived from Human Immunodeficiency Virus (HIV)); retroviral vectors (e.g. MMLV).

In some embodiments, the viral vector is an adeno-associated viral vector (AAV vector). In a preferred embodiment, the invention provides an AAV vector for use in treating or preventing a disease associated with Schwann cells, wherein the AAV vector comprises a first nucleic acid sequence that can be transcribed into a first polynucleotide.

It is preferred if the first nucleic acid sequence is transcribed, for example is transcribed in a target cell or target organism. Accordingly, a further embodiment provides a viral vector for use in treating or preventing a disease associated with Schwann cell wherein the viral vector comprises a first nucleic acid sequence that is transcribed into a first polynucleotide.

A further embodiment provides an AAV for use in treating or preventing a disease associated with Schwann cells wherein the viral vector comprises a first nucleic acid sequence that is transcribed into a first polynucleotide.

The first nucleic acid sequence may be transcribed into a first polynucleotide in a target cell or target organism, for example is transcribed in a Schwann cell. The Schwann cell may be in vivo, for example may be in a mammalian organism, which for example may be a human, cat, dog, mouse, rabbit, horse, for example.

Schwann cells are glial cells of the peripheral nervous system (PNS) that wrap around the axons of sensory and motor neurons and produce the surrounding myelin sheath. The myelin sheath is made up of several protein components (e.g. myelin protein zero) and is an essential insulating component of neurons that allows fast conduction of nervous impulses (action potentials) along nerves.

Some current viral-vector based therapeutic strategies utilize vectors that have undesirable characteristics. For example, some viral vectors integrate into the host genome with clear potential deleterious consequences. Accordingly, in one embodiment the viral vector is not a viral vector that integrates into the genome of the host cell, for example will not integrate into the nucleic acid of a Schwann cell. Viral vectors that are not considered to integrate into the host genome, in some embodiments are particularly preferred, and include AAVs and adenoviral vectors. AAV vectors infect target cells and the delivered genetic material does not integrate into the genome of the host cell. Instead, the delivered genetic material remains episomal.

Viral vectors that are considered to integrate into the host genome include the retroviral vectors, for example lentiviral vectors. Accordingly, in one embodiment the viral vector is not a vector that integrates into the host genome, for example is not a retroviral vector, for example is not a lentiviral vector.

Some vectors are also not able to transduce Schwann cells. The skilled person will understand the types of vectors that can and cannot transduce Schwann cells. Accordingly, in one embodiment the viral vector of the invention is not a viral vector that is unable to transduce Schwann cells. In some embodiments, the viral vector has the ability to transduce Schwann cells. By “transduce” we mean that the viral vector is capable of infecting the target cells and delivering the polynucleotide construct found within it into the target cell. Examples of such vectors include AAVs and lentiviral vectors.

In one embodiment the vector is a vector in which only an insert of limited size can be incorporated before becoming unstable. For example, such vectors include AAV vectors.

Preferably, the viral vector is an AAV vector and in some embodiments, the AAV vector is selected from the group comprising or consisting of: AAV9 and AAVrh10. In a particularly preferred embodiment, the AAV is an AAV9.

It is preferred that transcription of the first nucleic acid only occurs, or substantially only occurs in Schwann cells. Accordingly, in some embodiments, the viral vector also contains a Schwann cell specific promoter operably linked to the first nucleic acid.

By “Schwann cell specific promoter” we include the meaning of a promoter that results in significant expression in Schwann cells and no or low expression in non-Schwann cells. For example, a Schwann cell specific promoter may drive high levels of transcription from the first nucleic acid in Schwann cells (e.g. 95% or more total expression occurs in Schwann cells) whereas expression of the first polynucleotide is low in other cell types, for example those of the central nervous system (e.g. less than 5% of total expression occurs in cells other than Schwann cells). For example, in one embodiment, the ratio of transcription in Schwann cells to non-Schwann cell is at least 100:0; 95:5; 90:10; 85:15; 80:20; 75:25; 70:30; 65:35; 60:40; or 55:45.

In one embodiment the level of transcription in a Schwann cell is at least 1.5, 2, 3, 4, 5, 6, 7, 8, 9, 10, 20, 30, 40, 50, 60, 70, 80, 90, 100, 250, 500, 750, 1000, 2500, 5000, 7500, 10000 times higher than in any other non-Schwann cell.

In one embodiment a Schwann cell specific promoter results in the majority of expression occurring in Schwann cells rather than non-Schwann cells.

The skilled person will understand that even very specific promoters may result in some expression in other cells or tissues. The skilled person is well aware of the differential expression level between a target cell or tissue and a non-target cell or tissue that is required to classify a promoter as cell or tissue specific, for example Schwann cell specific. For example, (66) and (67) demonstrate the identification of cell-specific promoters in the central nervous system (CNS). The skilled person would be aware that for a promoter to be cell-specific it must contain regulatory elements that activate the promoter in certain cell types only (e.g. binding sites for transcription factors), and the promoter must be able to drive demonstrable expression of reporter genes or other genes in vitro and in vivo.

Preferably the Schwann cell specific promoter results in transcription of the first nucleic acid at a detectable level only in Schwann cells. The skilled person is well aware of routine methods to detect transcription, for example northern blot, PCR based techniques and immunofluorescence labelling. In one embodiment the Schwann cell specific promoter results in detectable transcription of the first nucleic acid in Schwann cells but does not result in detectable levels of transcription of the first nucleic acid in non-Schwann cells, for example in other cells of the peripheral nervous system or brain when the detection is performed using a northern blot analysis. In another embodiment the Schwann cell specific promoter results in detectable transcription of the first nucleic acid in Schwann cells but does not result in detectable levels of transcription of the first nucleic acid in non-Schwann cells, for example in other cells of the peripheral nervous system or brain when the detection is performed using an immunofluorescence labelling analysis with cell markers.

For example (32) and (33) demonstrate that Schwann cell specific expression can be achieved both in vitro and in vivo using constructs driven by the full-length Mpz promoter using lentiviral vectors.

Schwann cell specific promoters include, in some embodiments, myelin specific promoters. By “myelin specific promoter” we mean a promoter that typically drives the expression of genes encoding proteins making up the myelin sheath. Examples of myelin specific promoters include but are not limited to: the myelin protein zero (Mpz) promoter; the peripheral myelin protein 22 (PMP22) promoter; myelin associated glycoprotein (Mag) promoter.

In some embodiments, the expression of the first polynucleotide is under the control of a full-length myelin protein zero (Mpz) promoter, such as the full-length rat myelin protein zero (Mpz) promoter, the sequence of which is defined in SEQ ID NO. 4. In some embodiments, the sequence of the Mpz promoter has a sequence with at least 75% sequence homology or sequence identity with SEQ ID NO. 4, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 4.

In some embodiments, it will be clear to the skilled person that it is preferable for the promoter sequence to be derived from a human or humanised promoter sequence. In some embodiments the expression of the first polynucleotide is under the control of the full-length human myelin protein zero (hP0) promoter, the sequence of which is defined in SEQ ID NO. 18. In some embodiments, the sequence of the hP0 promoter has a sequence with at least 75% sequence homology or sequence identity with SEQ ID NO. 18, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 18.

As discussed above, it is considered advantageous if the promoter is as short as possible, particular where the vector is a vector that can only handle a limited insert size before becoming unstable. Therefore, in some embodiments, the expression of the first polynucleotide is under the control of a promoter that is between 100 bp and 1100 bp in length, optionally wherein the promoter ranges from 200 bp to 900 bp in length, 300 bp to 800 bp in length, 400 bp to 700 bp in length, optionally wherein the promoter is between 500 bp and 600 bp in length, for example is 410 bp in length. In the same or other embodiments, the promoter is less than 1100 bp in length, for example is less than 1000 bp, 900 bp, 800 bp, 700 bp, 600 bp, 500 bp, 400 bp, 300 bp, 200 bp or less than 100 bp in length.

In some embodiments the promoter is a naturally occurring Mpz promoter of a length as defined herein. In an alternative embodiment, the promoter is an engineered Mpz promoter of a length as defined herein. By “naturally occurring promoter” we mean a promoter that has not been modified, shortened or lengthened compared to the corresponding promoter sequence that is found in wild-type Schwann cells. By “engineered promoter”, we mean a wild-type promoter that has been altered in some way. For example, the sequence may have been modified to have for example at least 75% sequence homology or sequence identity with the naturally occurring promoter sequence, optionally at least 80%, or 82%, or 84%, or 86%, or 88%, or 90%, or 92%, or 94%, or 96%, or 97%, or 98%, or 99%, or 100% sequence identity or sequence homology to the naturally occurring promoter sequence. In another or the same embodiment, the promoter may also have been modified in length, for example the length of the wild-type promoter may have been reduced from a longer sequence to, for example between 100 bp and 1100 bp in length, optionally from 200 bp to 900 bp in length, 300 bp to 800 bp in length, 400 bp to 700 bp in length, optionally between 500 bp and 600 bp in length, for example is 410 bp in length, or is less than 1100 bp in length, for example is less than 1000 bp, 900 bp, 800 bp, 700 bp, 600 bp, 500 bp, 400 bp, 300 bp, 200 bp or less than 100 bp in length.

In another embodiment, the promoter length may have been increased relative to a wildtype promoter.

The skilled person will understand that it is possible that only portions of a particular nucleic acid region considered to be a promoter are actually required for promoter activity. In another example, the engineered promoter includes part of the sequence of the wild type promoter, or includes the whole sequence of the wild type promoter as part of a longer promoter sequence. As discussed, preferably the promoter is specifically active in Schwann cells. The skilled person would be able to test whether a particular fragment of a full-length promoter results in Schwann cell specific expression of a protein under control of said promoter fragment, for example by screening for expression of a reporter gene in Schwann cells. In some examples the reporter gene is EGFP.

In another embodiment, the engineered promoter is a truncated version of the wild type promoter and may have, for example, at least 75% sequence homology or sequence identity with the naturally occurring promoter sequence, optionally at least 80%, or 82%, or 84%, or 86%, or 88%, or 90%, or 92%, or 94%, or 96%, or 97%, or 98%, or 99%, or 100% sequence identity or sequence homology to the naturally occurring promoter sequence.

In addition to being a truncated version of a native promoter, the engineered promoter may additionally or alternatively comprise mutations, substitutions, deletions and insertions relative to the native promoter sequences. For example, an engineered promoter may comprise various different regions of the native promoter in one consecutive sequence.

An engineered promoter that is shorter in length than the corresponding native or wildtype promoter may be termed a minimal promoter.

In some embodiments, an engineered promoter retains the same function as the corresponding naturally occurring promoter that it is derived from i.e. it can still effectively drive transcription of polynucleotide sequences from nucleic acid sequences to which the promoter is operably linked and can in preferable instances effectively drive transcription in a cell-specific manner, i.e. in a Schwann cell specific manner.

In some embodiments the expression of the first polynucleotide may be under the control of, for example, a shortened naturally occurring myelin specific promoter, which is termed herein a minimal myelin specific promoter, optionally this is a minimal myelin protein zero (Mpz) promoter. In some embodiments the sequence of the minimal myelin specific promoter comprises or consists of the 410 bp sequence as defined in SEQ ID NO. 5, which is derived from the full length rat Mpz promoter sequence. In some embodiments, the minimal myelin specific promoter comprises or consists of a sequence with at least 75% sequence homology or sequence identity with SEQ ID NO. 5, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 5.

In some embodiments it is preferable for the minimal promoter to be derived from a human or humanised promoter sequence. In some embodiments the sequence of the minimal myelin specific promoter comprises or consists of the 429 bp sequence as defined in SEQ ID NO. 22, which is derived from the full length human hP0 promoter sequence. In some embodiments, the minimal myelin specific promoter comprises or consists of a sequence with at least 75% sequence homology or sequence identity with SEQ ID NO. 22, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 22. The minimal myelin specific promoters derived from rat Mpz or human hP0 are termed miniMpz herein.

By “sequence identity” or “sequence homology” we mean the identical sequence of base pairs in the specific DNA region. For example, in a sequence that has 75% sequence homology or sequence identity to a reference sequence, 75% of the base pairs are identical.

The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (Thompson et al., (1994) Nucleic Acids Res 22, 4673-80). The parameters used may be as follows: Fast pairwise alignment parameters: K-tuple(word) size; 1, window size; 5, gap penalty; 3, number of top diagonals; 5. Scoring method: x percent.

Multiple alignment parameters: gap open penalty; 10, gap extension penalty; 0.05.

Scoring matrix: BLOSUM.

In one embodiment, the minimal Mpz promoter described herein may be produced as described in Example 9, for example by taking the 410 base pair region upstream of the start codon of the full length promoter, for example the full length myelin protein zero (Mpz) promoter. In another embodiment a minimal Mpz promoter described herein may be produced as described in Example 13. AAV vectors have a maximum capacity to carry polynucleotides of around 4.4 kb, therefore the use of a shorter Mpz promoter as described herein, rather than the full length Mpz promoter which is approximately 1.1 kb in length, has the advantage of allowing longer first nucleic acid sequences to be operably linked to the promoter region for packaging into the AAV. In some embodiments where the promoter is a shorter promoter, for example with a length of between 100 bp and 1100 bp in length, 200 bp to 900 bp, 300 bp to 800 bp, 400 bp to 700 bp, 500 bp to 600 bp, or 410 bp in length, or with a length of less than 1100 bp in length, for example is less than 1000 bp, 900 bp, 800 bp, 700 bp, 600 bp, 500 bp, 400 bp, 300 bp, 200 bp or less than 100 bp, for example an engineered promoter or a minimal promoter, this allows the present invention to be applied to a wider range of genes of greater lengths than AAV vectors utilizing the full length promoter. For example, currently there are some situations where it is not possible to insert a nucleic acid sequence, for example a gene, of a particular length into an AAV since the length of the nucleic acid can exceed the maximum capacity of the AAV when longer promoters, for example the full length Mpz promoter is used. For example, if the first nucleic acid, for example the therapeutic gene, is longer than 3.0-3.3 kb in length (4.4 kb−1.1 kb=3.3 kb). In this case, use of the advantageous shorter promoters described herein, for example the minimal myelin specific promoter, allows the present invention to be applied to, for example, replacement of larger genes, such as the SH3TC2 gene which causes CMT type 4C (CMT4C), which is approximately 3.9 kb in length. Other Schwann cell-related genes that may be close to the stability limit of the AAV, and will therefore be optimally delivered under a minimal Mpz promoter, include EGR2 (2.98 kb) which is associated with CMT4E, and FGD4 (2.3 kb) which is associated with CMT4H.

In further additional embodiments, the vectors described herein can be modified in the inverted terminal repeat segment to further reduce their size. For example, the woodchuck hepatitis virus post-transcriptional regulatory element (WPRE), as described in Example 1, can be removed and/or the polyA sequence can be replaced with a minimal synthetic polyA (68, 69). Such modifications can further reduce the size of the vector to allow it to remail within the maximum capacity of the AAV and to allow efficient packaging when delivering larger genes. In further additional embodiments, the size of the vector can be further reduced by, for example, also using minimal versions of the protein coding gene to be delivered, wherein the minimal version of the protein coding gene is still able to produce functional protein.

In some embodiments, the viral vector described herein may be produced as described in Example 12. In some embodiments the viral vector has the sequence shown in SEQ ID NO: 20, which has the WPRE removed and has a synthetic polyA sequence. In some embodiments the synthetic polyA sequence comprises or consists of a minimum sequence required for efficient poyadenylation of mRNA constructs (68, 69). In some embodiments the synthetic polyA sequence comprises or consists of the sequence of SEQ ID NO: 24, which is included in the sequences of SEQ ID NO: 20 and 21. In other embodiments the synthetic polyA sequence has at least 80%, or 82%, or 84%, or 86%, or 88%, or 90%, or 92%, or 94%, or 96%, or 97%, or 98%, or 99%, or 100% sequence identity or sequence homology to SEQ ID NO. 24.

In some embodiments, the viral vector also contains binding sites for Egr2 and Sox10 transcription factors. For example, the viral vector may also contain enhancer elements to which transcription factors such as Egr2 and Sox10 can bind.

In some embodiments, the first nucleic acid of the viral vector is transcribed into a first polynucleotide that in some embodiments encodes and is translated into a first polypeptide or protein. In some embodiments, the first nucleic acid is the open reading frame (ORF) of a gene sequence or the cDNA corresponding to a gene sequence. In some embodiments, the first nucleic acid is the ORF or cDNA of a wild-type or other therapeutically beneficial gene sequence. In a preferred embodiment, the first nucleic acid is the ORF or cDNA of a wild-type or therapeutically beneficial sequence of a neuropathy-associated gene, optionally wherein the neuropathy is a demyelinating neuropathy.

By “wild type or therapeutically beneficial form” we include any form of the gene sequence that encodes a polypeptide or protein that can be used to effectively treat the disease associated with Schwann cells. The skilled person would understand that this would typically be the wild type form of the protein (i.e. that which is naturally occurring in Schwann cells) in situations where the disease arises through underproduction of the wildtype form of the polypeptide by the Schwann cells, but can also include forms of the protein that have mutations or insertions or are truncated compared to the wild type sequence to provide a therapeutic advantage, for example increased expression levels, resistance to degradation, increased stability, increased activity, or an advantageous gain of function, or to suppress a toxic gain of function. For example, in the latter case, the polypeptide may be an antibody capable of binding to the toxic gain of function mutant and suppress the toxicity.

The skilled person will be aware that protein expression is routinely carried out by introducing the ORF of the relevant gene, or the cDNA into the viral vector. In one embodiment the first nucleic acid is a cDNA sequence that when transcribed produces a first polynucleotide that is translated into a first polypeptide or protein. For example, the cDNA may be a cDNA sequence that is transcribed into GJB1 mRNA which is subsequently translated into Cx32 protein.

The skilled person will understand that the use of ORF sequences rather than cDNA sequences may be preferable in some instances, since the ORF sequence lacks additional non-coding elements found in cDNA and is smaller in size, which is particularly advantageous in the present invention when the viral vector is a vector that becomes unstable when the size increases above a particular threshold.

In some additional embodiments, the first nucleic acid sequence as described herein also optionally contains other regulatory elements in addition to the cDNA or ORF of the gene. These additional elements may be downstream of the ORF.

As discussed above, the invention has utility in the prevention or treatment of a disease associated with Schwann cells. By a “disease associated with Schwann cells” we include the meaning of all diseases that are associated with abnormal functioning of Schwann cells. This includes diseases associated with destruction of the myelin sheath formed by Schwann cells and/or diseases associated with reduced expression of myelin sheath formed by Schwann cells. In some embodiments, diseases associated with Schwann cells are demyelinating neuropathies. Examples of demyelinating neuropathies include but are not limited to Charcot-Marie-Tooth disease (CMT).

A “disease associated with Schwann cells” also includes in its meaning diseases that are associated with Schwann cells, but which are also associated with other cell types or tissues for example. It is considered that the invention is useful in such situations since an improvement in the function of Schwann cells can alleviate some symptoms, even if the invention does not target any other cell types that are associated with the disease.

Therefore, in one embodiment, the viral vectors described herein can be for use in treatment or prevention of a disease selected from the group consisting of: Charcot-Marie-Tooth disease (CMT); hereditary neuropathy with liability to pressure palsies (HNPP); diabetic and other toxic peripheral neuropathies; motor neuron disease (MND).

In some specific embodiments, the viral vectors described herein can be for use in treatment or prevention of Charcot-Marie-Tooth type 1X (CMT1X); Charcot-Marie-Tooth types 1A-1F (i.e. CMT1A, CMT1B, CMT1C, CMT1 D, CMT1E and CMT1 F); Charcot-Marie-Tooth types 4A-4H (i.e. CMT4A, CMT4B, CMT4C, CMT4D, CMT4E, CMT4F, CMT4G and CMT4H). In a more specific embodiment the viral vectors described herein can be used to treat or prevent Charcot-Marie-Tooth type 1X. In an alternative more specific embodiment, the viral vectors described herein can be used to treat or prevent Charcot-Marie-Tooth disease type 4C.

Charcot-Marie Tooth disease (CMT) is a group of demyelinating neuropathies caused by mutations in numerous different genes resulting in overlapping phenotypes. Charcot-Marie-Tooth type 1X (CMT1X) neuropathy is the second most common CMT form (4, 5) and presents with characteristic CMT1 symptoms, including progressive weakness and atrophy starting in distal leg muscles, difficulty running and frequently sprained ankles, with onset by 10 years of age or earlier in most affected males (6-8). The disease is slowly progressive causing weakness of foreleg muscles, foot drop, foot deformities, hand muscle weakness, and distal sensory loss with sometimes painful paresthesias by late adolescence or early adulthood and slow progression over the lifespan. Heterozygous females with CMT1X may be asymptomatic or develop milder clinical manifestations at an older age, but exceptionally severe neuropathy has been reported (9, 10). Transient CNS manifestations may occur in some, mostly younger CMT1X patients (11). Intermediate slowing (30-40 m/s) of motor nerve conduction velocities (MNCV) and progressive loss of motor units due to length-dependent axonal degeneration are typical electrophysiological features (6, 7). Nerve biopsies show mixed axonal and demyelinating abnormalities (12, 13) with thin myelin sheaths and loss of large myelinated fibers replaced by regenerating axon clusters (6, 14).

Cx32 is a transmembrane protein forming gap junction (GJ) channels through the non-compact myelin layers specifically expressed by myelinating Schwann cells in the peripheral nervous system (PNS), as well as by a subset of oligodendrocytes in the CNS. GJ channels formed by Cx32 serve important homeostatic and signaling functions that are essential for the function and survival of myelin and axons (4, 5). The corresponding gene that encodes Cx32 is GJB1.

More than 400 GJB1 mutations have been reported to date occurring throughout the open reading frame (ORF) and many in more than one family, including: 498 missense (71%); 3 stop-lost; 49 Inframe INDELs (7%); 25 Stop-Gained (4%); and 122 Frameshift INDELs (17%) (http://hihg.med.miami.edu/code/http/cmt/public_html/index.html #/). Several mutations have been reported also in non-coding GJB1 regions. Frameshift, premature stop and non-coding mutations are likely to cause complete loss of protein synthesis or rapid degradation, and are not expected to result in any dominant-negative effects. Several missense and in-frame mutations expressed in vitro showed intracellular retention (15-17) in the ER and/or Golgi (17-21) with failure to form functional channels. Some also exerted dominant-negative effects on co-expressed WT Cx32(15). Other mutants formed functional channels with altered biophysical characteristics (19). Cx32 knockout (KO) mice with complete deletion of the Gjb1/Cx32 gene develop a progressive, predominantly motor demyelinating peripheral neuropathy beginning at about three months of age with reduced sciatic MNCV and motor amplitude (24, 25). Expression of WT human Cx32 protein driven by the rat Mpz/P0 promoter prevented demyelination in Cx32 KO mice (26), confirming that loss of Schwann cell autonomous expression of Cx32 is sufficient to cause CMT1X pathology.

Thus, several in vitro and in vivo studies of CMT1X mutants support the overall conclusion that loss of Cx32 function mainly leads to the neuropathy in CMT1X (8, 17-19, 21-23). Accordingly, in one embodiment, gene replacement therapy using the viral vector and therapeutic methods as described herein is used to treat or prevent CMT1X, for example when the viral vector comprises a first nucleic acid sequences that encodes the wild type or therapeutically beneficial Cx32 protein.

Transgenic mice with mutations causing CMT1X on a KO background showed no detectable Cx32 protein in the 175fs mutant line (27), while R142W, T551, R75W and N175D transgenic mice showed retention of the mutant protein in the perinuclear region, similar to in vitro pattern (above) and developed a demyelinating neuropathy similar to Cx32 KO mice (22, 28, 29). In the presence of the Golgi-retained R142W, R75W and N175D mutants (but not of the ER-retained T551 mutant), there was reduced expression of the endogenous mouse WT Cx32, indicating that Golgi-retained mutants may have dominant-negative effects on WT Cx32. This is not clinically relevant for CMT1X patients expressing only one GJB1 allele in each cell, but must be considered when planning a gene addition therapy. None of the mutants expressed in vivo had any other toxic or dominant effects on other co-expressed connexins (22, 28). The C-terminus mutants C280G and S281X were properly localized and prevented demyelination in Cx32 KO mice, leaving unclear how they cause neuropathy in humans (30).

Accordingly, CMT1X may also be caused by dominant negative mutations in the Cx32 protein. In this instance the skilled person will understand that it is beneficial if the viral vector of the invention comprises a first nucleic acid that is transcribed into a non-coding RNA that itself is directed towards the mutant Cx32 mRNA to prevent translation of the mutant protein. The skilled person will understand how to arrive at suitable nucleic acid sequences that, for example, target the mutant Cx32 mRNA but not a wild-type or therapeutically advantageous Cx32 mRNA. In this way, in one embodiment, the subject may be treated with a viral vector that comprises a first nucleic acid that is transcribed into a non-coding RNA that targets the mutant Cx32, and the subject may also be treated with a second viral vector according to the invention wherein the second viral vector comprises a second nucleic acid that encodes the wild type or therapeutically advantageous Cx32 protein. In some embodiments, the first and second nucleic acid may be on the same viral vector according to the invention. A similar approach may be taken in the treatment or prevention of any Schwann cell associated disease as described herein.

In some embodiments, the first nucleic acid may be the ORF or cDNA of a wild-type gene sequence of a neuropathy associated gene. In some embodiments, the first nucleic acid may be the cDNA of a wild-type sequence of the gap junction beta 1 (GJB1) gene, which is considered to have a sequence as defined in SEQ ID NO. 6. In some embodiments, the first nucleic acid has at least 75% sequence homology or sequence identity with SEQ ID NO. 6, optionally at least 80%, or 82%, or 84%, or 86%, or 88%, or 90%, or 92%, or 94%, or 96%, or 97%, or 98%, or 99%, or 100% sequence identity or sequence homology with SEQ ID NO. 6. In other embodiments the first nucleic acid has at least 75% sequence homology or sequence identity with the ORF sequence of GJB1, optionally at least 80%, or 82%, or 84%, or 86%, or 88%, or 90%, or 92%, or 94%, or 96%, or 97%, or 98%, or 99%, or 100% sequence identity or sequence homology with the ORF sequence of GJB1.

CMT1X is caused by mutations in the GJB1 gene, causing under-expression of wild-type functional Cx32 protein. It follows that in some embodiments the viral vectors described herein may be for use in treatment or prevention of CMT1X by delivery of a wild-type copy or other therapeutically beneficial copy of the open reading frame or cDNA of the GJB1 gene.

Charcot-Marie-Tooth type 4C (CMT4C) disease is an autosomal recessive inherited neuropathy that appears to be the most prevalent among the overall rare recessive demyelinating CMT4 forms of neuropathies, being responsible for almost half of all CMT4 cases (35). Patients with CMT4C usually present in the first decade of life with foot deformities and scoliosis, weakness, areflexia and sensory loss (36-38). Cranial nerve involvement with hearing impairment, slow pupillary light reflexes, and lingual fasciculation are common and phenotypic variations in patients with identical mutations have been described (39-41). Electrophysiological studies in CMT4C patients confirm the demyelinating process with mean median motor nerve conduction velocity (NCV) at 22.6 m/s. Nerve biopsy findings are characterized by an increase of basal membranes around myelinated, demyelinated, and unmyelinated axons, relatively few onion bulbs, and, most typically, large cytoplasmic extensions of Schwann cells (36, 37, 42).

Molecular genetics of CMT4C: Linkage analysis studies and homozygosity mapping (43) led to the discovery of the disease locus on chromosome 5q32 and subsequently to the initial discovery of 11 different mutations in the SH3TC2 gene, mostly truncating but also missense (42). At least 28 different SH3TC2 mutations have been described to date, and they may be more common among certain ethnic groups (44) with likely founder effects (39). The full transcript cDNA length measures 3864 bp. SH3TC2 encodes a protein of 1,288 aa containing two Src homology 3 (SH3) and 10 tetratricopeptide repeat (TPR) domains sharing no overall significant similarity to any other human protein with known function. The presence of SH3 and TPR domains suggests that SH3TC2 could act as a scaffold protein (42). SH3TC2 is well conserved among vertebrate species, whereas no non-vertebrate orthologs were identified. SH3TC2 is present in several components of the endocytic pathway including early and late endosomes, and clathrin-coated vesicles close to the trans-Golgi network and in the plasma membrane. This localization is altered in CMT4C (45).

The Sh3tc2−/− KO mouse model of CMT4C develops an early onset but progressive peripheral neuropathy with slowing of motor and sensory nerve conduction velocities and early onset hypomyelination (46, 47). This phenotype is progressive with increasing myelin pathology at 2 and 12 months of age. Murine Sh3tc2 is specifically expressed in Schwann cells and is localized to the plasma membrane and to the perinuclear endocytic recycling compartment, suggesting a possible function in myelination and/or in regions of axoglial interactions (48). Ultrastructural analysis of myelin in the peripheral nerve of mutant mice showed abnormal organization of the node of Ranvier, a phenotype that was confirmed in nerve biopsies from CMT4C patients. These findings suggested a role for the SH3TC2 gene product in myelination and in the integrity of the node of Ranvier (46). Thus, the Sh3tc2−/− mouse recapitulates all major features of CMT4C disease and provides a relevant model to test therapies.

Therefore, in some embodiments, the first nucleic acid may be the ORF or cDNA of the wild-type sequence of the gene SH3 domain and tetratricopeptide repeats 2 (SH3TC2) gene. The ORF of SH3TC2 is considered to have a sequence as defined in SEQ ID NO. 7. In some embodiments, the first nucleic acid has at least 75% sequence homology or sequence identity with SEQ ID NO. 7, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 7. In other embodiments the first nucleic acid has at least 75% sequence homology or sequence identity with the cDNA sequence of SH3TC2, optionally at least 80%, or 82%, or 84%, or 86%, or 88%, or 90%, or 92%, or 94%, or 96%, or 97%, or 98%, or 99%, or 100% sequence identity or sequence homology with the cDNA sequence of SH3TC2.

As discussed above, CMT4C is caused by mutations in the SH3TC2 gene, causing under-expression of wild-type functional SH3TC2 protein. It follows that in some embodiments the viral vectors described herein may be for use in treatment or prevention of CMT4C by delivery of a wild-type copy or other therapeutically beneficial copy of the open reading frame or cDNA of SH3TC2, to, for example, increase expression of the wildtype SH3TC2.

In another embodiment, the viral vectors described herein can be used in methods of treatment or prevention of other types of autosomal dominant demyelinating CMT.

CMT1B is caused by mutations in the myelin protein zero (Mpz) gene, causing under-expression of wild-type functional Mpz protein.

Therefore, in some embodiments, the first nucleic acid may be the ORF or cDNA of the wild-type sequence of the myelin protein zero (MPZ) gene. The ORF of MPZ is considered to have a sequence as defined in SEQ ID NO. 9. In some embodiments, the first nucleic acid has at least 75% sequence homology or sequence identity with SEQ ID NO. 9, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 9. In other embodiments the first nucleic acid has at least 75% sequence homology or sequence identity with the cDNA sequence of MPZ, optionally at least 80%, or 82%, or 84%, or 86%, or 88%, or 90%, or 92%, or 94%, or 96%, or 97%, or 98%, or 99%, or 100% sequence identity or sequence homology with the cDNA sequence of MPZ.

It follows that in some embodiments the viral vectors described herein may be for use in treatment or prevention of CMT1B by delivery of non-coding RNAs as described herein targeting and knocking down toxic mutant alleles of the MPZ gene in addition to delivering a wild-type copy or other therapeutically beneficial copy of the open reading frame or cDNA of the MPZ gene.

CMT1 D is caused by mutations in the EGR2 gene, causing under-expression of wild-type functional EGR2 protein.

Therefore, in some embodiments, the first nucleic acid may be the ORF or cDNA of the wild-type sequence of the early growth response 2 (EGR2) gene. The ORF of EGR2 is considered to have a sequence as defined in SEQ ID NO. 10. In some embodiments, the first nucleic acid has at least 75% sequence homology or sequence identity with SEQ ID NO. 10, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 10. In other embodiments the first nucleic acid has at least 75% sequence homology or sequence identity with the cDNA sequence of EGR2, optionally at least 80%, or 82%, or 84%, or 86%, or 88%, or 90%, or 92%, or 94%, or 96%, or 97%, or 98%, or 99%, or 100% sequence identity or sequence homology with the cDNA sequence of EGR2.

It follows that in some embodiments the viral vectors described herein may be for use in treatment or prevention of CMT1 D by delivery of a wild-type copy or other therapeutically beneficial copy of the open reading frame or cDNA of the EGR2 gene.

In another embodiment, the viral vectors described herein can be used in methods of treatment or prevention of other types of autosomal recessive demyelinating CMT. CMT4A is caused by mutations in the GDAP1 gene, causing under-expression of wild-type functional GDAP1 protein.

Therefore, in some embodiments, the first nucleic acid may be the ORF of the wild-type sequence of the ganglioside induced differentiation associated protein 1 (GDAP1) gene. The ORF of GDAP1 is considered to have a sequence as defined in SEQ ID NO. 11. In some embodiments, the first nucleic acid has at least 75% sequence homology or sequence identity with SEQ ID NO. 11, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 11. In other embodiments the first nucleic acid has at least 75% sequence homology or sequence identity with the cDNA sequence of GDAP1, optionally at least 80%, or 82%, or 84%, or 86%, or 88%, or 90%, or 92%, or 94%, or 96%, or 97%, or 98%, or 99%, or 100% sequence identity or sequence homology with the cDNA sequence of GDAP1.

It follows that in some embodiments, the viral vectors described herein may be for use in treatment or prevention of CMT4A by delivery of a wild-type copy or other therapeutically beneficial copy of the open reading frame or cDNA of the GDAP1 gene.

CMT4D is caused by mutations in the NDRG1 gene, causing under-expression of wild-type functional NDRG1 protein.

Therefore, in some embodiments, the first nucleic acid may be the ORF or cDNA of the wild-type sequence of the N-Myc downstream regulated 1 (NDRG1) gene. The ORF of NDRG1 is considered to have a sequence as defined in SEQ ID NO. 12. In some embodiments, the first nucleic acid has at least 75% sequence homology or sequence identity with SEQ ID NO. 12, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 12. In other embodiments the first nucleic acid has at least 75% sequence homology or sequence identity with the cDNA sequence of NDRG1, optionally at least 80%, or 82%, or 84%, or 86%, or 88%, or 90%, or 92%, or 94%, or 96%, or 97%, or 98%, or 99%, or 100% sequence identity or sequence homology with the cDNA sequence of NDRG1.

It follows that in some embodiments the viral vectors described herein may be for use in treatment or prevention of CMT4D by delivery of a wild-type copy or other therapeutically beneficial copy of the open reading frame or cDNA of the NDRG1 gene.

CMT4E is caused by mutations in the EGR2 gene, causing under-expression of wild-type functional EGR2 protein.

Therefore, in some embodiments, the first nucleic acid may be the ORF or cDNA of the wild-type sequence of the early growth response 2 (EGR2) gene. The ORF of EGR2 is considered to have a sequence as defined in SEQ ID NO. 10. In some embodiments, the first nucleic acid has at least 75% sequence homology or sequence identity with SEQ ID NO. 10, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 10. In other embodiments the first nucleic acid has at least 75% sequence homology or sequence identity with the cDNA sequence of EGR2, optionally at least 80%, or 82%, or 84%, or 86%, or 88%, or 90%, or 92%, or 94%, or 96%, or 97%, or 98%, or 99%, or 100% sequence identity or sequence homology with the cDNA sequence of EGR2.

It follows that in some embodiments the viral vectors described herein may be for use in treatment or prevention of CMT4E by delivery of a wild-type copy or other therapeutically beneficial copy of the open reading frame or cDNA of the EGR2 gene.

Hereditary neuropathy with liability to pressure palsies (HNPP) is associated with a mutation in the PMP22 gene, causing under-expression of wild-type functional PMP22 protein.

Therefore, in some embodiments, the first nucleic acid may be the ORF or cDNA of the wild-type sequence of the peripheral myelin protein 22 (PMP22) gene. The ORF of PMP22 is considered to have a sequence as defined in SEQ ID NO. 8. In some embodiments, the first nucleic acid has at least 75% sequence homology or sequence identity with SEQ ID NO. 8, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 8. In other embodiments the first nucleic acid has at least 75% sequence homology or sequence identity with the cDNA sequence of PMP22, optionally at least 80%, or 82%, or 84%, or 86%, or 88%, or 90%, or 92%, or 94%, or 96%, or 97%, or 98%, or 99%, or 100% sequence identity or sequence homology with the cDNA sequence of PMP22.

It follows that in some embodiments, the viral vectors described herein may be for use in treatment or prevention of HNPP by delivery of a wild-type copy or other therapeutically beneficial copy of the open reading frame or cDNA of the PMP22 gene.

In another embodiment, the first nucleic acid may be the ORF or cDNA of another gene associated with a demyelinating neuropathy and/or Schwann cell dysfunction. It follows that, in some embodiments, the viral vectors described herein may be for use in treatment or prevention of diseases associated with a demyelinating neuropathy and/or Schwann cell dysfunction by delivery of a wild-type copy or other therapeutically beneficial copy of the open reading frame or cDNA of a gene associated with such a disease.

Motor neuron disease (MND) (also called amyotrophic lateral sclerosis) is a neurodegenerative disorder with complex causes that have not been fully determined. In some embodiments, the viral vectors described herein may be used to deliver polynucleotides encoding trophic factors (for example brain-derived neurotrophic factor (BDNF), glial cell-derived neurotrophic factor (GDNF), neurotrophin-3 (NT-3), vascular endothelial growth factor (VEGF)). Expression of such trophic factors in target cells is considered to be useful in regenerating and saving stressed motor neurons.

It follows that in some embodiments, the viral vectors described herein are for use in methods of treating or preventing MND.

It would be clear to the skilled person that the wild-type or therapeutically beneficial form of the proteins disclosed herein could be expressed either from the nucleotide sequence of the full gene, just from the open reading frame sequence (ORF), or just from the cDNA sequence. All of these types of sequences would be readily accessed by the skilled person from a sequence database e.g. GenBank (accessible here: https://www.ncbi.nlm.nih.gov/genbank/).

In some embodiments, the first polynucleotide encodes and is translated into a first polypeptide or protein. In some embodiments, the first polynucleotide encodes a wild-type form of a protein. In some embodiments, the wild-type form of the protein is used to replace or supplement expression of a mutant form of the same protein that is expressed by a subject in need thereof.

In some embodiments, the first polynucleotide may encode a wild-type or therapeutically beneficial form of one or more of the following proteins: connexin-32 (Cx32); SH3 domain and tetratricopeptide repeats 2 (SH3TC2); peripheral myelin protein 22 (PMP22); myelin protein zero (MPZ); early growth response 2 (EGR2); ganglioside induced differentiation associated protein 1 (GDAP1); N-Myc downstream regulated 1 (NDRG1). The skilled person would understand that the amino acid sequences of the proteins disclosed herein could be readily accessed from a sequence database e.g. the NCBI Protein Database (accessible here: https://www.ncbi.nlm.nih.gov/protein).

Therefore, in some embodiments the invention can be applied to methods of gene replacement by providing an AAV vector containing a wild-type form or other therapeutically beneficial form of a gene to be replaced. In some non-limiting examples, the gene to be replaced may be mutated in such a way that it does not encode protein, it encodes a truncated version of the wild-type protein (for example there is a premature stop codon), it encodes a reduced amount of functional protein or it encodes a non-functional mutant form of the protein.

In an additional or alternative embodiment, the first nucleic acid encodes and is translated into a trophic factor (for example brain-derived neurotrophic factor (BDNF), glial cell-derived neurotrophic factor (GDNF), neurotrophin-3 (NT-3), vascular endothelial growth factor (VEGF)). By trophic factor we include biomolecules (for example proteins or peptides) which support the growth, differentiation and/or development of developing and mature neurons. In another additional or alternative embodiment, the first polynucleotide encodes a regenerative factor (for example Angiogenin, Oct-6, Egr2, Sox-10). In another additional or alternative embodiment, the first polynucleotide encodes a growth factor (for example IGF).

Using the vectors described herein to deliver nucleic acids encoding the trophic factors, regenerative factors and/or growth factors described above may be used in some embodiments to treat or prevent acquired peripheral nerve disorders. In one example, diabetic and other toxic peripheral neuropathies could be treated by delivering vectors as described herein encoding trophic factors and/or growth factors to Schwann cells and axons. In another example, motor neuron disease (MND) (also known as amyotrophic lateral sclerosis) may be treated by delivering vectors as described herein encoding trophic factors that can be delivered to axons of stressed motor neurons to retroactively save said motor neurons.

In another embodiment, the administration of a viral vector comprising a first nucleic acid that encodes a first protein or polypeptide leads to improved functioning of Schwann cells and/or increased formation of myelin sheath. In some embodiments this improvement in function is achieved by increased formation of myelin sheath by Schwann cells when compared to the formation of myelin sheath by Schwann cells in the subject prior to treatment and the improvement in function can be detected via the detection of an increased production of myelin sheath. In some embodiments, the improvement in function can be measured by an improvement in muscle strength and/or improved sciatic nerve conduction velocity and/or changes to potential response of blood biomarkers when compared to these measures in the subject prior to treatment. The skilled person is aware of techniques to determine an improvement in the function of Schwann cells and/or increased formation of myelin sheath. Some such techniques are provided in the Examples.

In some specific embodiments, the increased formation of myelin sheath by the Schwann cells leads to improved myelination of the peripheral nerves. By improved myelination of the peripheral nerves we mean that there is increased myelination of peripheral nerves compared to the subject before treatment. This includes a decrease in demyelinated and remyelinated fibers and/or a reduction in abnormally myelinated fibers. Improved myelination may also be associated with a reduction in the number of foamy macrophages, which is a marker of inflammation, in some embodiments. Improved myelination may also be associated with increased myelin thickness and reduced g-ratios (axonal diameter divided by myelinated fiber diameter).

As described above, the first nucleic acid may encode a polypeptide or protein that has therapeutic benefits, for example when the native protein is mutated or expressed at a level which is too low to result in normal functionality.

It will be appreciated that in an alternative embodiment, the first nucleic acid may be transcribed into an RNA that is not an mRNA, i.e. is not an RNA that is translated into a protein. Accordingly, the first nucleic acid may be transcribed into a non-coding RNA.

By “non-coding RNA” we mean any RNA molecule that is not translated into a polypeptide or protein. The skilled person will be aware of such RNA polymers and how they can be used to affect the expression of polypeptides. In one embodiment the first nucleic acid is transcribed into a non-coding micro-RNA (miR). In a further additional alternative embodiment, the first nucleic acid is transcribed into a short-hairpin RNA (shRNA). In a further embodiment the first nucleic acid is transcribed into a guide RNA (gRNA), for example as part of a CRISPR-based system.

Expression of the non-coding RNA described above when the viral vector is in a target organism may lead to reduction in expression of a target polynucleotide, optionally wherein the target polynucleotide is a gene located in a target organism, optionally wherein it is located in a cell in a target organism. In some embodiments the target polynucleotide is a gene sequence. Therefore, in some embodiments the invention described herein can be used to knock-down expression of a target gene. By “knock-down” we mean that the expression of the target gene is reduced compared to expression levels prior to treatment with the viral vector.

For example, the invention can be applied to situations where a target nucleic acid, for example a target gene, is over-expressed. The viral vector can be used to deliver a first nucleic acid that is transcribed into a non-coding RNA to, for example, target the mRNA produced by the gene that this over-expressed for degradation (e.g. by the RISC complex, which is well known in the art) or to directly block translation of said mRNA into protein.

This embodiment of the invention also applies to situations in which the target nucleic acid is itself transcribed into a non-coding RNA, and it is beneficial to reduce the levels of the host non-coding RNA in the cell.

This embodiment of the invention can also be used to target deleterious gain-of-function mutants and reduce their protein or mRNA expression levels.

Therefore, in some embodiments, expression of non-coding RNA results in reduction in expression of a target nucleic acid, polynucleotide or gene. In one embodiment expression or overexpression of the target polynucleotide in a target organism is considered to be associated with a disease associated with Schwann cells, optionally wherein the disease is a dominant demyelinating neuropathy (CMT1), optionally wherein the target polynucleotide is a mutated allele of myelin protein zero (Mpz/P0) and the disease associated with Schwann cells is CMT1B, or wherein the target polynucleotide is another dominant gene associated with CMT1.

In some embodiments, the administration of a viral vector encoding a first nucleic acid results in expression of a non-coding RNA that leads to improved functioning of Schwann cells. As discussed above, in some embodiments this improvement in function is achieved by increased formation of myelin sheath by Schwann cells when compared to the formation of myelin sheath by Schwann cells in the subject prior to treatment. In some embodiments, this improvement in function can be measured by an improvement in muscle strength and/or improved sciatic nerve conduction velocity and/or changes to potential response of blood biomarkers when compared to these measures in the subject prior to treatment.

In some embodiments, the viral vectors described herein comprises a first nucleic acid sequence that encodes a first polypeptide or protein, and the vector can also comprise a second nucleic acid that is transcribed into a non-coding RNA. Therefore, the invention can be used in some embodiments to knock-down expression of a mutant gene using a non-coding RNA and to also replace the mutant gene with a wild-type copy of said gene, resulting in complete gene replacement. This approach is considered to be particularly useful where the subject in need of therapy has a gain of function mutation in a particular protein.

In some embodiments, the viral vector also contains a second or third nucleic acid sequence that encodes a transcription factor capable to driving expression or increased expression from the Schwann cell specific promoter, optionally the myelin specific promoter or minimal myelin specific promoters as defined herein. Examples of such transcription factors that can drive expression of polynucleotides under the control of Schwann cell specific promoters include Egr2 and Sox10.

The viral vector may also comprise a nucleic acid sequence that encodes a Cas9 polypeptide or similar that is routinely used in CRISPR techniques and variations thereof, such as dead-Cas9.

It will be understood that the viral vectors described herein can be administered to the subject in a variety of ways. In a preferred embodiment, the viral vectors described herein are administered by intrathecal injection. By “intrathecal injection” we include injection into the spinal canal which results in the injected material reaching the cerebrospinal fluid (CSF). In a particularly preferred embodiment, the viral vectors described herein are administered by lumbar intrathecal injection. The viral vectors described herein are also suitable for administration by thoracic intrathecal injection or cervical intrathecal injection. Alternatively, the viral vectors described herein could be administered by direct injection into peripheral nerves. Alternatively, the viral vectors described herein could be administered by direct intravenous injection.

Intrathecal injection provides advantages over other administration methods such as intraneural and intraveneous injection. Compared to intraneural injection, intrathecal injection provides a more widespread distribution to multiple spinal roots and nerves. In contrast, intraneural injection provides distribution only within the injected nerve. In addition, intrathecal injection can be done routinely in the clinic, does not require surgical procedure and is considered safe, while intraneural injections will require surgical procedure, and multiple nerves to be exposed, higher risk, so much more difficult to translate in the clinic.

While intravenous injection is easier to administer in the clinic, it has the disadvantage of requiring much higher doses of the vector to reach the nervous system compared to intrathecal delivery. Intravenous delivery can also lead to more toxicity, due to higher doses of the virus and liver toxicity risk. In addition, intravenous injection is likely to cause more immune reactions, whereas intrathecal delivery provides a possibility to evade the immune system with lower immune response.

Once the AAV vectors described herein have transduced the target cell the genetic material that is delivered remains stable and episomal, providing the target cell has differentiated and is not dividing, as is the case with mature Schwann cells. Therefore, a single administration of an AAV vector should be sufficient to achieve therapeutic effects, and in some embodiments the viral vectors described herein are administered by a single intrathecal injection. However, in some cases, it may be necessary to administer multiple doses of different AAV vectors to the subject at different time points. These different vectors may express different first polynucleotides, or may express the same first polynucleotide and differ in the type of AAV that is used. Therefore, in some embodiments the viral vectors disclosed herein may be useful in treating or preventing diseases associated with Schwann cells that are associated with multiple different genes.

It will be appreciated that the viral vectors disclosed herein are suitable for use in human subjects. The viral vectors are also suitable for use in mammals in general such as: cat, dog, mouse, rabbit, horse. The subjects may be treated with the viral vectors disclosed herein either prior to the onset of symptoms of the disease associated with Schwann cells or after the onset of symptoms of said disease. The subjects to be treated may be any age at the onset of treatment. For example, the subjects may be treated with the vector(s) of the invention as soon as it is confirmed that the subject has a mutation or other defect compromising performance of the Schwann cells. This may be before any symptoms are exhibited.

It will be appreciated that the dose of viral vector used will be adjusted according to the requirement of the subject in need thereof, for example it may be adjusted due to the age, weight or height of the subject. As a general example, a dose of (for intrathecal delivery) escalating doses at 3.5×10¹³ vector genomes (vg), 3.3× higher dose of 1.2×10¹⁴ vg, and the 5 times higher dose of 1.8×10¹⁴ vg, could be used. Doses such as these have been used previously in clinical trials using AAVs (e.g. https://clinicaltrials.gov/ct2/show/NCT02362438), and the skilled person would be aware that they could be applied to the present invention.

It will be clear to the skilled person that in addition to therapeutic methods of preventing or treating a disease associate with Schwann cells, the invention also provides the viral vector per se. Accordingly, in another aspect, the invention provides a viral vector as described herein comprising a nucleic acid sequence as defined herein. In a preferred embodiment the viral vector is an AAV. In a particularly preferred embodiment the AAV is an AAV9. Preferences for features of this aspect are as described elsewhere in this specification, for example the preferences for the vector, nucleic acid, promoter, Schwann cell associated disease are as defined herein.

In a further aspect, the invention provides a minimal myelin specific promoter comprising or consisting of the sequence as defined in SEQ ID NO. 5 or a sequence with at least 75% sequence homology or sequence identity with SEQ ID NO. 5, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 5. Preferences for features of this aspect are as described in this specification, for example the preferences for the vector, nucleic acid, promoter, Schwann cell associated disease are as defined herein.

In a further aspect, the invention provides a minimal myelin specific promoter comprising or consisting of the sequence as defined in SEQ ID NO. 22 or a sequence with at least 75% sequence homology or sequence identity with SEQ ID NO. 22, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 22. Preferences for features of this aspect are as described in this specification, for example the preferences for the vector, nucleic acid, promoter, Schwann cell associated disease are as defined herein. In some embodiments, the invention provides a human minimal myelin specific promoter, wherein the human minimal myelin specific promoter has a sequence homology with at least 75%, 80%, or 82%, or 84%, or 86%, or 88%, or 90%, or 92%, or 94%, or 96%, or 97%, or 98%, or 99%, or 100% sequence homology or sequence identity with SEQ ID NO. 22.

In a further aspect, the invention provides a polynucleotide construct comprising a first nucleic acid sequence that is a Schwann cell specific promoter, optionally a myelin specific promoter, optionally comprising the myelin protein zero (Mpz) promoter or a minimal myelin specific promoter as defined herein, operably linked to a second nucleic acid sequence which is transcribed into a first polynucleotide, wherein the second nucleic acid: a) is the open reading frame or cDNA or other elements of a gene; or b) is transcribed into a non-coding RNA.

The invention also provides a viral vector comprising such a polynucleotide construct, for example provides an AAV vector comprising the construct. Preferences for features of this aspect are as described in this specification, for example the preferences for the vector, nucleic acid, promoter, Schwann cell associated disease are as defined herein. For example in one embodiment the polynucleotide construct of the invention comprises a Schwann cell specific promoter, wherein the promoter is a) a minimal Schwann cell specific promoter, optionally a minimal Mpz promoter as described herein, for example where the promoter has a sequence with at least 75%, 80%, or 82%, or 84%, or 86%, or 88%, or 90%, or 92%, or 94%, or 96%, or 97%, or 98%, or 99%, or 100% sequence homology or sequence identity with SEQ ID NO. 5 or SEQ ID NO. 22; or b) a full-length Mpz promoter optionally wherein the promoter has a sequence with at least 75%, 80%, or 82%, or 84%, or 86%, or 88%, or 90%, or 92%, or 94%, or 96%, or 97%, or 98%, or 99%, or 100% sequence homology or sequence identity with SEQ ID NO. 4 or SEQ ID NO. 18.

Preferably the polynucleotide construct of the invention comprises a human minimal Mpz or human full-length Mpz promoter as described herein.

In a further aspect, the invention provides the following viral vectors:
a) An AAV-Mpz.Egfp vector comprising an AAV9 vector, the myelin protein zero (Mpz) promoter and the EGFP reporter gene (SEQ ID NO. 1), optionally wherein the promoter has a sequence with at least 75%, 80%, or 82%, or 84%, or 86%, or 88%, or 90%, or 92%, or 94%, or 96%, or 97%, or 98%, or 99%, or 100% sequence homology or sequence identity with SEQ ID NO. 4 or SEQ ID NO. 18;
b) An AAV9-Mpz-GJB1 vector comprising an AAV9 vector, the myelin protein zero (Mpz) promoter and the open reading frame (ORF) of the gap junction beta 1 (GJB1) gene (SEQ ID NO. 2), optionally wherein the promoter has a sequence with at least 75%, 80%, or 82%, or 84%, or 86%, or 88%, or 90%, or 92%, or 94%, or 96%, or 97%, or 98%, or 99%, or 100% sequence homology or sequence identity with SEQ ID NO. 4 or SEQ ID NO. 18;
c) An AAV9-miniMpz.Egfp vector comprising an AAV9 vector, the minimal myelin protein zero (miniMpz) promoter and the EGFP reporter gene (SEQ ID NO. 3), optionally wherein the miniMPZ promoter has a sequence homology with at least 75%, 80%, or 82%, or 84%, or 86%, or 88%, or 90%, or 92%, or 94%, or 96%, or 97%, or 98%, or 99%, or 100% sequence homology or sequence identity with SEQ ID NO. 5 or SEQ ID NO. 22;
d) An AAV9-human Mpz-GJB1 vector comprising an AAV9 vector, the human myelin protein zero (hP0) promoter and the open reading frame (ORF) of the gap junction beta 1 (GJB1) gene (SEQ ID NO. 17);
e) An AAV9-human Mpz-Egfp vector comprising an AAV9 vector, the human myelin protein zero (hP0) promoter and the EGFP reporter gene (SEQ ID NO. 19);
f) An AAV9-miniMpz-SH3TC2.myc.ITR vector comprising an AAV9 vector, a minimal myelin protein zero (Mpz) promoter and the open reading frame (ORF) of the SH3TC2 gene (SEQ ID NO. 20);
g) An AAV9-human-miniMpz-SH3TC2 vector comprising an AAV9 vector, a human minimal myelin protein zero (hP0) promoter and the open reading fram (ORF) of the SH3TC2 gene (SEQ ID NO. 21);
h) An AAV9-human-miniMpz-Egfp vector comprising an AAV9 vector, a human minimal myelin protein zero (hP0) promoter and the EGFP reporter gene (SEQ ID NO. 23); or i) an AAV, optionally wherein the AAV vector is an AAV9;

Preferences for features of this aspect are as described in this specification, for example the preferences for the vector, nucleic acid, promoter, Schwann cell associated disease are as defined herein.

In one specific embodiment, the invention also provides a viral vector for use in treating or preventing a disease associated with Schwann cells in a subject in need thereof, wherein the viral vector comprises a first nucleic acid sequence that is transcribed into a first polynucleotide, and wherein transcription of said first nucleic acid is under the control of a minimal myelin specific promoter, optionally comprising or consisting of the sequence defined in SEQ ID NO. 5 or SEQ ID NO. 22 or that has at least 75% sequence homology or sequence identity with SEQ ID NO. 5 or 22, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 5 or 22. In one embodiment, the viral vector may be an AAV vector. In another alternative embodiment, the viral vector may be a lentiviral vector. Preferences for features of this aspect are as described in this specification, for example the preferences for the vector, nucleic acid, promoter, Schwann cell associated disease are as defined herein.

In another aspect, the invention also provides pharmaceutical compositions comprising any of the viral vectors as described herein. In some embodiments, the pharmaceutical composition comprises an appropriate amount of the viral vector and further comprises a pharmaceutically acceptable excipient, diluent, carrier, buffer or adjuvant. Preferences for features of this aspect are as described in this specification, for example the preferences for the vector, nucleic acid, promoter, Schwann cell associated disease are as defined herein.

As used herein, “pharmaceutical composition” means a therapeutically effective formulation for use in the treatment or prevention of diseases associated with Schwann cells.

The pharmaceutical compositions may be prepared in a manner known in the art that is sufficiently storage stable and suitable for administration to humans.

By “pharmaceutically acceptable” we mean a non-toxic material that does not decrease the effectiveness of the biological activity of the active ingredients, i.e. the viral vector. Such pharmaceutically acceptable carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), which are incorporated herein by reference).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to mean an aqueous or non-aqueous solution with the purpose of diluting the viral vector in the pharmaceutical preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the viral vector. The adjuvant may be one or more of colloidal silver, or zinc, copper or silver salts with different anions, for example, but not limited to fluoride, chloride, bromide, iodide, tiocyanate, sulfite, hydroxide, phosphate, carbonate, lactate, glycolate, citrate, borate, tartrate, and acetates of different acyl composition. The adjuvant may also be cationic polymers such as PHMB, cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, sucrose, mannitol, and cyclodextrines, which are added to the composition, e.g., for facilitating lyophilisation. Examples of polymers are starch, cellulose ethers, cellulose, carboxymethylcellulose, hydroxypropylmethyl cellulose, hydroxyethyl cellulose, ethylhydroxyethyl cellulose, ethyl cellulose, methyl cellulose, propyl cellulose, alginates, carageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, poly(lactic acid), poly(glycholic acid) or copolymers thereof with various composition, and polyvinylpyrrolidone, all of different molecular weight, which are added to the composition, e.g. for viscosity control, for achieving bioadhesion, or for protecting the active ingredient from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

In another aspect the invention provides the use of a viral vector as described herein in a method of manufacture of a medicament for the treatment or prevention of a disease associated with Schwann cells. In some embodiments the disease causes destruction and/or reduced formation of myelin sheath by Schwann cells. In a preferred embodiment the disease is Charcot-Marie-Tooth disease. Preferences for features of this aspect are as described in this specification, for example the preferences for the vector, nucleic acid, promoter, Schwann cell associated disease are as defined herein.

In yet another aspect, the invention provides methods of treatment or prevention of a disease associated with Schwann cells using any of the viral vectors described herein. In a specific embodiment, the invention provides methods of treatment or prevention of Charcot-Marie-Tooth disease. In a preferred embodiment, the disease is Charcot-Marie-Tooth disease type 1X or type 4C. Preferences for features of this aspect are as described in this specification, for example the preferences for the vector, nucleic acid, promoter, Schwann cell associated disease are as defined herein.

The skilled person will appreciate that the viral vectors described herein could be used in a CRISPR/Cas system for use in gene editing or gene silencing, for example by using a dead-Cas9 polypeptide. Accordingly, in another aspect the invention includes a viral vector or polynucleotide construct as described herein for use in a CRISPR/Cas9 system comprising any one or more of:
a) a polynucleotide encoding a single guide RNA (sgRNA) targeting a gene of interest;
b) a polynucleotide encoding a Cas9 polypeptide;
c) a polynucleotide encoding a polypeptide of interest.

Preferences for features of this aspect are as described in this specification, for example the preferences for the vector, nucleic acid, promoter, Schwann cell associated disease are as defined herein.

It would be clear to the skilled person that the viral vectors disclosed herein could have a variety of uses other than for the treatment or prevention of diseases associated with Schwann cells. For example, the viral vectors disclosed herein may be used in a method of labelling Schwann cells, for example with fluorescent protein, for example green fluorescent protein (GFP) or enhanced green fluorescent protein (EGFP), or with other non-fluorescent reporters. In some examples, the labelling of Schwann cells can be used in a method of diagnosing a disease associated with Schwann cells.

Preferences for features of this aspect are as described in this specification, for example the preferences for the vector, nucleic acid, promoter, Schwann cell associated disease are as defined herein.

In another example, the viral vectors disclosed herein may be used in methods of inducing Schwann cells to differentiate into alternative cell types, for example neurons, oligodednrocytes, or astrocytes.

In yet another example, the viral vectors disclosed herein may be used in methods of stimulating Schwann cells to support regeneration in a subject in need thereof, for example after an injury or trauma. Preferences for features of this aspect are as described in this specification, for example the preferences for the vector, nucleic acid, promoter, Schwann cell associated disease are as defined herein.

In yet another example, the viral vectors disclosed herein may be suitable for use in ex vivo methods of treating diseases associated with Schwann cells. For example, target cells could be removed from the subject in need of treatment and transduced with a viral vector as described herein before being introduced back into the subject.

Preferences for features of this aspect are as described in this specification, for example the preferences for the vector, nucleic acid, promoter, Schwann cell associated disease are as defined herein.

The invention also provides a cell that has been transduced by the viral vector of the invention, for example a Schwann cell.

The invention also provides a cell that comprises the nucleic acid construct of the invention that comprises the relevant promoter and first nucleic acid. The skilled person would be aware that the viral vectors of the present invention can be produced in cultured cells, preferably HEK293 cells, for example as described in (58).

It will be appreciated that the vectors and methods described herein can be performed in vivo, but may also be used ex vivo or in vitro, for example cells such as Schwann cells may be transduced in vitro or ex vivo for subsequent therapeutic or research purposes.

The invention also provides kits that can be used to implement any of the viral vectors described herein. For example, the invention provides a kit for use with the viral vector or polynucleotide of any of the preceding claims wherein the kit comprises one or more of:
a) a viral vector as defined herein;
b) a polynucleotide construct as defined herein;
c) a viral vector;
d) a viral vector comprising the polynucleotide construct as defined herein;
e) a pharmaceutically acceptable carrier and/or excipient;
f) a single-use syringe, for example a single-use syringe suitable for intrathecal lumbar injection;
g) instructions for use.

In one embodiment the kit comprises more than on viral vector according to the invention, for example the kit may comprise two different viral vectors as defined herein.

It will be clear to the skilled person that in any of the therapeutic uses of the invention, more than one viral vector according to the invention may be administered to the subject. It will be clear to the skilled person that in some situations this is advantageous, for example if more than one gene is known to be associated with the Schwann cell associated disease, multiple viral vectors may be administered, each vector directed towards expressing a different therapeutic protein. Alternatively, a single vector may express more than one therapeutic protein or non-coding RNA. in other situations, such as those described above, one viral vector can be used to express for example a Cas9 protein in Schwann cells, and a different viral vector can be used to express the relevant gRNA to target Cas9 to the required nucleic acid.

The listing or discussion of an apparently prior published document in this specification should not necessarily be taken as an acknowledgement that the document is part of the state of the art or is common general knowledge.

Preferences and options for a given aspect, feature or parameter of the invention should, unless the context indicates otherwise, be regarded as having been disclosed in combination with any and all preferences and options for all other aspects, features and parameters of the invention.

Accordingly, and to exemplify how the disclosure of one aspect of the invention relates to other aspects of the invention, and to demonstrate how these aspects may be combined, the invention, in some embodiments, provides:

A viral vector for use in the treatment or prevention of a disease associated with Schwann cells wherein the viral vector is an AAV and wherein the viral vector comprises a first nucleic acid that can be transcribed into a first polynucleotide, wherein expression of the first polynucleotide is under the control of a minimal myelin specific (Mpz) promoter; A viral vector for use in the treatment or prevention of a disease associated with Schwann cells wherein the viral vector is an AAV and wherein the viral vector comprises a first nucleic acid that can be transcribed into a first polynucleotide, wherein expression of the first polynucleotide is under the control of a) a myelin protein zero (Mpz) promoter, optionally wherein the promoter has a sequence with at least 75%, 80%, or 82%, or 84%, or 86%, or 88%, or 90%, or 92%, or 94%, or 96%, or 97%, or 98%, or 99%, or 100% sequence homology or sequence identity with SEQ ID NO. 4 or SEQ ID NO. 1; or
b) a minimal myelin specific promoter (miniMpz), optionally comprising or consisting of the sequence defined in SEQ ID NO. 5 or SEQ ID NO. 22, optionally wherein the miniMPZ promoter has a sequence homology with at least 75%, 80%, or 82%, or 84%, or 86%, or 88%, or 90%, or 92%, or 94%, or 96%, or 97%, or 98%, or 99%, or 100% sequence homology or sequence identity with SEQ ID NO. 5 or SEQ ID NO. 22;

A polynucleotide construct comprising a first nucleic acid sequence that is a minimal myelin specific (Mpz) promoter which is operably linked to a second nucleic acid sequence, wherein the second nucleic acid sequence is either the open reading frame of a gene sequence or encodes a non-coding RNA;

A minimal myelin specific (Mpz) promoter that drives high levels of expression in Schwann cells and is suitable for use in the viral vectors described herein.

The invention also provides:
a viral vector for use in treating or preventing CMT1X, wherein the vector comprises a human Mpz promoter (according to SEQ ID NO: 18) operably linked to the GJB1 gene, wherein the viral vector is AAV9;
use of a viral vector in a method of manufacture of a medicament for the treatment or prevention of CMT1X, wherein the vector comprises a human Mpz promoter (according to SEQ ID NO: 18) operably linked to the GJB1 gene, where the viral vector is AAV9; and
a method of treating or preventing CMT1X wherein the method comprises administering a viral vector to a patient in need thereof, wherein the viral vector comprises a human Mpz promoter (according to SEQ ID NO: 18) operably linked to the GJB1 gene, and where the viral vector is AAV9.

The invention also provides:
a viral vector for use in treating or preventing CMT4C, wherein the vector comprises the human minimal Mpz promoter (according to SEQ ID NO: 22) operably linked to the SH3TC2 gene, where the viral vector is AAV9;
   use of a viral vector in a method of manufacture of a medicament for the treatment of prevention of CMT4C, wherein the vector comprises a human Mpz promoter (according to SEQ ID NO: 22) operably linked to the SH3TC2 gene, where the viral vector is AAV9; and
a method of treating or preventing CMT4C wherein the method comprises administering a viral vector to a patient in need thereof, wherein the viral vector comprises a human Mpz promoter (according to SEQ ID NO: 22) operably linked to the SH3TC2 gene, and where the viral vector is AAV9.

A patient in need thereof includes a patient that has displayed symptoms or has otherwise received a diagnosis of one of the diseases disclosed herein, and also includes a patient that is suspected of having, or will develop, one of the diseases disclosed herein.

### DESCRIPTION OF THE FIGURES

FIG. 1: AAV vector transfer plasmids generated for Schwann cell-targeted gene expression: pAAV-Mbz.GJB1 vector containing the human GJB1 open reading frame expressing Cx32 (Full) and pAAV-Mpz.Egfp expressing the reporter gene EGFP (Mock).

FIG. 2: AAV9-mediated Schwann-cell targeted gene expression. A-D: Four weeks following lumbar intrathecal (i.th.) injection of the AAV9-Mpz-Egfp vector in 2-month old wild-type (WT) mice, immunostaining of lumbar root sections (A-B) with EGFP antibody (A2, B) shows perinuclear expression (asterisks) in a subset of Schwann cells at low (left) and higher (right) magnification. EGFP expression is also seen in the sciatic nerve section at low magnification without antibody staining (C2) and at higher magnification of teased sciatic nerve fibers immunostained with EGFP antibody (D2). A1, C1, and D1 show only nuclear staining with DAPI of the same areas shown in A2, C2, D2. E: Quantification of EGFP-positive Schwann cell ratios in lumbar roots and sciatic nerves. F: Vector copy numbers (VCNs) in lumbar roots, proximal and distal sections of the sciatic nerves demonstrate a gradient of biodistribution of the vector towards peripheral nerves after intrathecal injection. G: Immunoblot analysis of lumbar root (LR), femoral nerves (FN) and sciatic nerves (SN) lysates from different mice (1-4) shows the specific EGFP specific band in most of the tissue of injected mice corresponding to the positive control (+) from a transgenic sample, while it is absent in negative (−) control (Kagiava et al., unpublished).

FIG. 3: Expression of intrathecally delivered AAV9-Mpz.GJB1 vector in 2-month Cx32KO and R75W KO mice. A. Vector copy numbers (VCN) in relevant tissues. Immunostaining of WT (B) and Cx32 KO (C) sciatic teased fibers demonstrates the specific Cx32 localization at paranodal myelin areas in the WT fiber (arrows) which is absent in the Cx32 KO. AAV9-Mpz.GJB1 i.th. injection results in paranodal Cx32 expression not only in Cx32 KO sciatic fibers (D), but also in R75W KO fibers (E), despite the presence of R75W mutant in perinuclear areas (asterisk and open arrowheads). F: Western blot analysis of Cx32 expression in lumbar root and sciatic nerve samples (TG+: transgenic-positive; KO: untreated Cx32 KO-negative controls) (Kagiava et al., unpublished).

FIG. 4: Behavioral analysis of AAV9-Mpz.GJB1 (full) injected post-onset at 6 months of age Cx32 KO mice compared to AAV9-Mpz.Egfp (mock) treated littermates. Results of rotarod (left) and foot grip (right) testing of motor performance in AAV9-Mpz.GJB1 treated (GJB1) compared to mock treated Cx32 KO mice, as indicated. Time course analysis of each group showed improved motor performance of fully treated Cx32 KO mice in rotarod and foot grip analysis 2 months post-injection (8 months of age) and then motor performance remained stable up to 10 months of age. In contrast, mock treated mice did not improve over time as indicated by both behavioral tests.

FIG. 5: Results of sciatic nerve motor conduction studies. Motor nerve conduction velocities (MNCV) were improved in the 10-month old fully treated Cx32 KO mice compared to the mock vector injected littermates approaching the values of WT.

FIG. 6: Morphological analysis of anterior spinal roots of Cx32 KO mice following post-onset intrathecal delivery of the AAV9-Mpz.GJB1 compared to mock-treated mice vector. Representative images of semithin sections of anterior lumbar spinal roots attached to the spinal cord at low (A-B) and higher (C-D) magnification, as well as morphometric analysis results (E-F) from mock or full (GJB1) vector treated mice as indicated, at 10 months of age (4 months after treatment). AAV9-Mpz.GJB1 injected mouse roots (B, D) show improved myelination compared with roots of a mock-treated littermate (A, C) with fewer demyelinated (*) and remyelinated (r) fibers. Quantification of the ratios of abnormally myelinated fibers in multiple roots confirms significant improvement in the numbers of abnormally myelinated fibers (E) as well as significant reduction in the numbers of foamy macrophages (F) in fully treated compared to mock vector treated littermates.

FIG. 7: Morphological analysis of sciatic nerves of Cx32 KO mice following post-onset intrathecal delivery of the AAV9-Mpz.GJB1 vector. Representative images of semithin sections of sciatic nerves at low (A-B) and higher (C-D) magnification, as well as morphometric analysis results (E-F) from mock or full (GJB1) vector treated mice as indicated, at 10 months of age (4 months after treatment). AAV9-Mpz.GJB1 injected mouse nerves (B, D) show improved myelination compared with nerves of a mock-treated littermate (A, C) with fewer demyelinated (*) and remyelinated (r) fibers. Quantification of the ratios of abnormally myelinated fibers in multiple nerves confirms significant improvement in the numbers of abnormally myelinated fibers (E) as well as significant reduction in the numbers of foamy macrophages (F) in fully treated compared to mock vector treated littermates.

FIG. 8: Morphological analysis of femoral nerves of Cx32 KO mice following post-onset intrathecal delivery of the AAV9-Mpz.GJB1 vector. Representative images of semithin sections of femoral nerves at low (A-B) and higher (C-D) magnification, as well as morphometric analysis results (E-F) from mock or full (GJB1) vector treated mice as indicated, at 10 months of age (4 months after treatment). AAV9-Mpz.GJB1 injected mouse nerves (B, D) show improved myelination compared with nerves of a mock-treated littermate (A, C) with fewer demyelinated (*) and remyelinated (r) fibers. Quantification of the ratios of abnormally myelinated fibers in multiple nerves confirms significant improvement in the numbers of abnormally myelinated fibers (E) as well as significant reduction in the numbers of foamy macrophages (F) in fully treated compared to mock vector treated littermates.

FIG. 9: The miniMpz-Egfp construct cloned into the AAV transfer plasmid after PCR amplification of a 410 bp sequence from the 1127 bp full-length rat Mpz promoter.

FIG. 10: Immunostaining of lumbar root and sciatic nerve longitudinal sections 4 weeks following lumbar intrathecal injection of the AAV9-miniMpz-Egfp vector in 2-mo old WT mice with EGFP antibody shows perinuclear expression in a subset of Schwann cells (A, C). B and C are negative controls showing only the nuclear staining with DAPI. E: Percentage of EGFP-positive Schwann cells (n=5-6 mice). F: Vector copy numbers in lumbar roots and sciatic nerves demonstrate adequate biodistribution of the vector after intrathecal injection (n=6 mice).

FIG. 11: Minimal CNS expression of the AAV9-miniMpz-Egfp vector. Immunostaining of lumbar spinal cord longitudinal sections 4 weeks following lumbar intrathecal injection of the AAV9-miniMpz-Egfp vector in 2-mo old WT mice with EGFP antibody in combination with cell markers NeuN (A, labelling neurons), GFAP (B, labeling astrocytes), CC-1 (C-D, labeling oligodendrocytes) shows that only a few cells of each cell type express EGFP (examples indicated by arrows) while most are EGFP-negative (examples are indicated by open arrowheads). E. Quantification in n=3-5 mice per cell marker staining shows low expression rates in all three CNS cell types of around 2-3%.

FIG. 12: Motor behavioural testing in groups of Cx32 KO mice (CMT1X model) treated pre-onset at the age of 2 months with either the full therapeutic (AAV9-Mpz-GJB1) vector or the mock vector (AAV9-Mpz-Egfp). Foot grip strength testing was carried out before treatment (2 months of age) and at 4 (FIG. 12A) and 6 (FIG. 12B) months of age. There is significant functional improvement in the treated groups at 4 and 6 months. FIG. 12C shows a significant improvement over time following treatment, whereas mock treated mice did not show any improvement.

FIG. 13: Electrophysiological studies of pre-onset treated (full) and mock-treated 6-month old Cx32 KO mice. Sciatic motor nerve conduction studies were carried out at 6 months of age, and showed significant improvement of sciatic nerve conduction velocities after gene therapy treatment at the age of 2 months, compared to the mock treatment.

FIG. 14: Morphological analysis of anterior (motor) lumbar roots of 6 month old Cx32 KO mice following pre-onset treatment with either the full therapeutic (AAV9-Mpz-GJB1) vector or the mock vector (AAV9-Mpz-Egfp) at 2 months old. Representative images of semithin sections of anterior (motor) lumbar roots. AAV9-Mpz-GJB1 treated mice (B, D) show improved myelination compared to mock treated mice (A, C) with fewer demyelinated (*) and remyelinated (r) fibers. As confirmed by quantitative analysis (E, F), fewer demyelinated (*) or remyelimated (r) fibers (E) and fewer foamy macrphages (F) were found in treated compared to mock treated mice.

FIG. 15: Morphological analysis of mid-sciatic nerves of 6 month old Cx32 KO mice following pre-onset treatment with either the full therapeutic (AAV9-Mpz-GJB1) vector or the mock vector (AAV9-Mpz-Egfp) at 2 months old. Representative images of semithin sections of mid-sciatic nerves. AAV9-Mpz-GJB1 treated mice (B, D) show improved myelination compared to mock treated mice (A, C) with fewer demyelinated (*) and remyelinated (r) fibers. As confirmed by quantitative analysis (E, F), fewer demyelinated (*) or remyelimated (r) fibers (E) and fewer foamy macrphages (F) were found in treated compared to mock treated mice.

FIG. 16: Morphological analysis of femoral motor nerves of 6 month old Cx32 KO mice following pre-onset treatment with either the full therapeutic (AAV9-Mpz-GJB1) vector or the mock vector (AAV9-Mpz-Egfp) at 2 months old. Representative images of semithin sections of femoral motor nerves. AAV9-Mpz-GJB1 treated mice (B, D) show improved myelination compared to mock treated mice (A, C) with fewer demyelinated (*) and remyelinated (r) fibers. As confirmed by quantitative analysis (E, F), fewer demyelinated (*) or remyelimated (r) fibers (E) and fewer foamy macrphages (F) were found in treated compared to mock treated mice.

FIG. 17: Expression analysis of SH3TC2 in peripheral nervous system of Sh3tc2−/− mice following intrathecal delivery of novel therapeutic vector AAV9-mini-Mpz-SH3TC2.myc. Expression of human normal SH3TC2 protein (red) mainly in the perinuclear cytoplasm of myelinating Schwann cells in lumbar roots (A) and sciatic nerves (section in D and teased fibers in F) 4 weeks following intrathecal injection of the AAV9-miniMpz-SH3TC2myc vector into Sh3tc2−/− mice. Tissues of non-injected mice are shown in A, C, E as negative control. Cell nuclei are stained blue. Quantification of the expression rates (% SH3TC2-positive cells) in lumbar roots and sciatic nerves in n=5 mice is shown in FIG. 17G.

Sequences

SEQ ID NO. 1: AAV-Mpz.Egfp construct
tagctgcgcgctcgctcgctcactgaggccgcccgggcaaagcccgggcgtcgggcgacctttggtcgcccggcctcagt
gagcgagcgagcgcgcagagagggagtggccaactccatcactaggggttccttgtagttaatgattaacccgccatgcta
cttatctacgtagccatgctctaggtaccgggccccccctcgaggtcgacggtatcgataagcttcctgttcagactcgtttcctgctgtac
cctttcaatggccccacatcaaatcaaacacagatggcacatatctactctaaatatatgcagagcttcacaaacgtcatacacgtacgtg
tgtcacacacgcacacacacacccttccacctctgcccttacctttgctgtcccatctagacattatccctcccatccccttatttcccttatcaa
aatggctgctccttcaaggttccaaataacactgcttcctggacctgactcctctttcctctgaacttcctgtgttaagtgtattcctagtgcactg
tgccttggtagttgttgagattgccctctgcttctcccttctgcctcctcatctagtgatcttgagcttgtagaaagaactgaattaccattctaata
cgagcattctcgaactctccaaatagccaccaagcaggacaataggcagtcttgatcatttaaactgctgcatggcaaaaggaatcgaa
ggatttcttaacagaagtgggggggggggagatctgggcttcttcctggaagtttcctgatagagaaaatcttctgcctgggtagaatctcc
caggatgcagggagatggaaaaagtgttccccaaggactttgtagtctacaggttgtggagccatcggaacaacgagacaccctaattt
gggagtgctctgaaagaaacttgcctctaggccctagggctctcaggcaaggaggctaagaaggaatcctttgctgtagccttttggattt
aggtttctcagcttatctatccctcagagaagtgtgtctatgtcccttttctgtccctctgcctcaccccaccccaacattccaacctagggtag
ggggaggtcagtatacacaaagccctctgtgtaaggggtggtatgtgtccccccacccccctacccagagtatacaatgccccttctgctc
catgcccctgccaccctcccaccacctctcaattgcacatgccaggctgcaattggtcactggctcaggacagccccctcatgctgggga
tccaggggattttaagcaggttccagaaaacaccactcagttccttgtcccccgctctctccaccccacagacgctctgccaagcttgatat
cgaattgatccaccggtcgccaccatggtgagcaagggcgaggagctgttcaccggggtggtgcccatcctggtcgagctggacggcg
acgtaaacggccacaagttcagcgtgtccggcgagggcgagggcgatgccacctacggcaagctgaccctgaagttcatctgcacca
ccggcaagctgcccgtgccctggcccaccctcgtgaccaccctgacctacggcgtgcagtgcttcagccgctaccccgaccacatgaa
gcagcacgacttcttcaagtccgccatgcccgaaggctacgtccaggagcgcaccatcttcttcaaggacgacggcaactacaagacc
cgcgccgaggtgaagttcgagggcgacaccctggtgaaccgcatcgagctgaagggcatcgacttcaaggaggacggcaacatcct
ggggcacaagctggagtacaactacaacagccacaacgtctatatcatggccgacaagcagaagaacggcatcaaggtgaacttca
agatccgccacaacatcgaggacggcagcgtgcagctcgccgaccactaccagcagaacacccccatcggcgacggccccgtgct
gctgcccgacaaccactacctgagcacccagtccgccctgagcaaagaccccaacgagaagcgcgatcacatggtcctgctggagtt
cgtgaccgccgccgggatcactctcggcatggacgagctgtacaagtaaagcggccctagatcaagcttatcgataatcaacctctgga
ttacaaaatttgtgaaagattgactggtattcttaactatgttgctccttttacgctatgtggatacgctgctttaatgcctttgtatcatgctattgctt
cccgtatggctttcattttctcctccttgtataaatcctggttgctgtctctttatgaggagttgtggcccgttgtcaggcaacgtggcgtggtgtgc
actgtgtttgctgacgcaacccccactggttggggcattgccaccacctgtcagctcctttccgggactttcgctttccccctccctattgccac
ggcggaactcatcgccgcctgccttgcccgctgctggacaggggctcggctgttgggcactgacaattccgtggtgttgtcggggaaatc
atcgtcctttccttggctgctcgcctgtgttgccacctggattctgcgcgggacgtccttctgctacgtcccttcggccctcaatccagcggacc
ttccttcccgcggcctgctgccggctctgcggcctcttccgcgtcttcgccttcgccctcagacgagtcggatctccctttgggccgcctcccc
gcatcgataccgtcgactcgctgatcagcctcgactgtgccttctagttgccagccatctgttgtttgcccctcccccgtgccttccttgaccct
ggaaggtgccactcccactgtcctttcctaataaaatgaggaaattgcatcgcattgtctgagtaggtgtcattctattctggggggtggggt
ggggcaggacagcaagggggaggattgggaagacaatagcaggcatgctggggatgcggtgggctctatggcttctgaggcggaa
agaaccagctggggctcgactagagcatggctacgtagataagtagcatggcgggttaatcattaactacaaggaacccctagt
gatggagttggccactccctctctgcgcgctcgctcgctcactgaggccgggcgaccaaaggtcgcccgacgcccgggctt
tgcccgggcggcctcagtgagcgagcgagcgcgcagagctttttgcaaaagcctaggcctccaaaaaagcctcctcactacttct
ggaatagctcagaggccgaggcggcctcggcctctgcataaataaaaaaaattagtcagccatggggcggagaatgggcggaactg
ggcggagttaggggcgggatgggcggagttaggggcgggactatggttgctgactaattgagatgcatgctttgcatacttctgcctgctg
gggagcctggggactttccacacctggttgctgactaattgagatgcatgctttgcatacttctgcctgctggggagcctggggactttccac
accctaactgacacacattccacagctgcattaatgaatcggccaacgcgcggggagaggcggtttgcgtattgggcgctcttccgcttc
ctcgctcactgactcgctgcgctcggtcgttcggctgcggcgagcggtatcagctcactcaaaggcggtaatacggttatccacagaatc
aggggataacgcaggaaagaacatgtgagcaaaaggccagcaaaaggccaggaaccgtaaaaaggccgcgttgctggcgtttttc
cataggctccgcccccctgacgagcatcacaaaaatcgacgctcaagtcagaggtggcgaaacccgacaggactataaagatacca
ggcgtttccccctggaagctccctcgtgcgctctcctgttccgaccctgccgcttaccggatacctgtccgcctttctcccttcgggaagcgtg
gcgctttctcatagctcacgctgtaggtatctcagttcggtgtaggtcgttcgctccaagctgggctgtgtgcacgaaccccccgttcagccc
gaccgctgcgccttatccggtaactatcgtcttgagtccaacccggtaagacacgacttatcgccactggcagcagccactggtaacag
gattagcagagcgaggtatgtaggcggtgctacagagttcttgaagtggtggcctaactacggctacactagaagaacagtatttggtatc
tgcgctctgctgaagccagttaccttcggaaaaagagttggtagctcttgatccggcaaacaaaccaccgctggtagcggtggtttttttgttt
gcaagcagcagattacgcgcagaaaaaaaggatctcaagaagatcdttgatcttttctacggggtctgacgctcagtggaacgaaaa
ctcacgttaagggattttggtcatgagattatcaaaaaggatcttcacctagatcdtttaaattaaaaatgaagttttaaatcaatctaaagta
tatatgagtaaacttggtctgacagttaccaatgcttaatcagtgaggcacctatctcagcgatctgtctatttcgttcatccatagttgcctgac
tccccgtcgtgtagataactacgatacgggagggcttaccatctggccccagtgctgcaatgataccgcgagacccacgctcaccggct
ccagatttatcagcaataaaccagccagccggaagggccgagcgcagaagtggtcctgcaactttatccgcctccatccagtctattaat
tgttgccgggaagctagagtaagtagttcgccagttaatagtttgcgcaacgttgttgccattgctacaggcatcgtggtgtcacgctcgtcgt
ttggtatggcttcattcagctccggttcccaacgatcaaggcgagttacatgatcccccatgttgtgcaaaaaagcggttagctccttcggtc
ctccgatcgttgtcagaagtaagttggccgcagtgttatcactcatggttatggcagcactgcataattctcttactgtcatgccatccgtaag
atgcttttctgtgactggtgagtactcaaccaagtcattctgagaatagtgtatgcggcgaccgagttgctcttgcccggcgtcaatacggga
taataccgcgccacatagcagaactttaaaagtgctcatcattggaaaacgttcttcggggcgaaaactctcaaggatcttaccgctgttg
agatccagttcgatgtaacccactcgtgcacccaactgatcttcagcatcttttactttcaccagcgtttctgggtgagcaaaaacaggaag
gcaaaatgccgcaaaaaagggaataagggcgacacggaaatgttgaatactcatactcttcctttttcaatattattgaagcatttatcagg
gttattgtctcatgagcggatacatatttgaatgtatttagaaaaataaacaaataggggttccgcgcacatttccccgaaaagtgccacct
gacgtctaagaaaccattattatcatgacattaacctataaaaataggcgtatcacgaggccctttcgtctcgcgcgtttcggtgatgacggt
gaaaacctctgacacatgcagctcccggagacggtcacagcttgtctgtaagcggatgccgggagcagacaagcccgtcagggcgc
gtcagcgggtgttggcgggtgtcggggctggcttaactatgcggcatcagagcagattgtactgagagtgcaccattcgacgctctccctt
atgcgactcctgcattaggaagcagcccagtagtaggttgaggccgttgagcaccgccgccgcaaggaatggtgcatgcaaggagat
ggcgcccaacagtcccccggccacggggcctgccaccatacccacgccgaaacaagcgctcatgagcccgaagtggcgagcccg
atcttccccatcggtgatgtcggcgatataggcgccagcaaccgcacctgtggcgccggtgatgccggccacgatgcgtccggcgtag
aggatctggctagcgatgaccctgctgattggttcgctgaccatttccgggtgcgggacggcgttaccagaaactcagaaggttcgtcca
accaaaccgactctgacggcagtttacgagagagatgatagggtctgcttcagtaagccagatgctacacaattaggcttgtacatattgt
cgttagaacgcggctacaattaatacataaccttatgtatcatacacatacgatttaggtgacactatagaatacacggaattaattc
Bold = ITR sequence
Italics = Mpz promoter
Underline = EGFP
Italic underline = WPRE sequence
SEQ ID NO. 2: AAV-Mpz.GJB1 construct
tagctgcgcgctcgctcgctcactgaggccgcccgggcaaagcccgggcgtcgggcgacctttggtcgcccggcctcagt
gagcgagcgagcgcgcagagagggagtggccaactccatcactaggggttccttgtagttaatgattaacccgccatgcta
cttatctacgtagccatgctctaggtacccctcgaagcttcctgttcagactcgtttcctgctgtaccattcaatggccccacatcaaatca
aacacagatggcacatatctactctaaatatatgcagagcttcacaaacgtcatacacgtacgtgtgtcacacacgcacacacacaccc
ttccacctctgcccttacctttgctgtcccatctagacattatccctcccatccccttatttcccttatcaaaatggctgctccttcaaggttccaaa
taacactgcttcctggacctgactcctctttcctctgaacttcctgtgttaagtgtattcctagtgcactgtgccttggtagttgttgagattgccctc
tgcttctcccttctgcctcctcatctagtgatcttgagcttgtagaaagaactgaattaccattctaatacgagcattctcgaactctccaaatag
ccaccaagcaggacaataggcagtcttgatcatttaaactgctgcatggcaaaaggaatcgaaggatttcttaacagaagtggggggg
ggggagatctgggcttcttcctggaagtttcctgatagagaaaatcttctgcctgggtagaatctcccaggatgcagggagatggaaaaa
gtgttccccaaggactttgtagtctacaggttgtggagccatcggaacaacgagacaccctaatttgggagtgctctgaaagaaacttgcc
tctaggccctagggctctcaggcaaggaggctaagaaggaatcctttgctgtagccttttggatttaggtttctcagcttatctatccctcaga
gaagtgtgtctatgtcccttttctgtccctctgcctcaccccaccccaacattccaacctagggtagggggaggtcagtatacacaaagccc
tctgtgtaaggggtggtatgtgtccccccacccccctacccagagtatacaatgccccttctgctccatgcccctgccaccctcccaccacc
tctcaattgcacatgccaggctgcaattggtcactggctcaggacagccccctcatgctggggatccaggggattttaagcaggttccaga
aaacaccactcagttccttgtcccccgctctctccaccccacagacgctctgccaagcttcgagaatgaggcaggatgaactggacagg
tttgtacaccttgctcagtggcgtgaaccggcattctactgccattggccgagtatggctctcggtcatcttcatcttcagaatcatggtgctggt
ggtggctgcagagagtgtgtggggtgatgagaaatcttccttcatctgcaacacactccagcctggctgcaacagcgtttgctatgaccaa
ttcttccccatctcccatgtgcggctgtggtccctgcagctcatcctagtttccaccccagctctcctcgtggccatgcacgtggctcaccagc
aacacatagagaagaaaatgctacggcttgagggccatggggaccccctacacctggaggaggtgaagaggcacaaggtccacat
ctcagggacactgtggtggacctatgtcatcagcgtggtgttccggctgttgtttgaggccgtcttcatgtatgtcttttatctgctctaccctggc
tatgccatggtgcggctggtcaagtgcgacgtctacccctgccccaacacagtggactgcttcgtgtcccgccccaccgagaaaaccgt
cttcaccgtcttcatgctagctgcctctggcatctgcatcatcctcaatgtggccgaggtggtgtacctcatcatccgggcctgtgcccgccg
agcccagcgccgctccaatccaccttcccgcaagggctcgggcttcggccaccgcctctcacctgaatacaagcagaatgagatcaac
aagctgctgagtgagcaggatggctccctgaaagacatactgcgccgcagccctggcaccggggctgggctggctgaaaagagcga
ccgctgctcggcctgctgaggatccctcgaggtcgacggtatcgataagcttatcgataatcaacctctggattacaaaatttgtgaaagat
tgactggtattcttaactatgttgctccttttacgctatgtggatacgctgctttaatgcctttgtatcatgctattgcttcccgtatggctttcattttctc
ctccttgtataaatcctggttgctgtctctttatgaggagttgtggcccgttgtcaggcaacgtggcgtggtgtgcactgtgtttgctgacgcaac
ccccactggttggggcattgccaccacctgtcagctcctttccgggactttcgctttccccctccctattgccacggeggaactcatcgccgc
ctgccttgcccgctgctggacaggggctcggctgttgggcactgacaattccgtggtgttgtcggggaaatcatcgtcctttccttggctgctc
gcctgtgttgccacctggattctgcgcgggacgtccttctgctacgtcccttcggccctcaatccagcggaccttccttcccgcggcctgctg
ccggctctgcggcctcttccgcgtcttcgccttcgccctcagacgagtcggatctccctttgggccgcctccccgcatcgataccgtcgactc
gctgatcagcctcgactgtgccttctagttgccagccatctgttgtttgcccctcccccgtgccttccttgaccctggaaggtgccactcccact
gtcctttcctaataaaatgaggaaattgcatcgcattgtctgagtaggtgtcattctattctggggggtggggtggggcaggacagcaagg
gggaggattgggaagacaatagcaggcatgctggggatgcggtgggctctatggcttctgaggcggaaagaaccagctggggctcga
ctagagcatggctacgtagataagtagcatggcgggttaatcattaactacaaggaacccctagtgatggagttggccactcc
ctctctgcgcgctcgctcgctcactgaggccgggcgaccaaaggtcgcccgacgcccgggctttgcccgggcggcctcag
tgagcgagcgagcgcgcagagctttttgcaaaagcctaggcctccaaaaaagcctcctcactacttctggaatagctcagaggccg
aggcggcctcggcctctgcataaataaaaaaaattagtcagccatggggcggagaatgggcggaactgggcggagttaggggcggg
atgggcggagttaggggcgggactatggttgctgactaattgagatgcatgctttgcatacttctgcctgctggggagcctggggactttcc
acacctggttgctgactaattgagatgcatgctttgcatacttctgcctgctggggagcctggggactttccacaccctaactgacacacatt
ccacagctgcattaatgaatcggccaacgcgcggggagaggcggtttgcgtattgggcgctcttccgcttcctcgctcactgactcgctgc
gctcggtcgttcggctgcggcgagcggtatcagctcactcaaaggcggtaatacggttatccacagaatcaggggataacgcaggaaa
gaacatgtgagcaaaaggccagcaaaaggccaggaaccgtaaaaaggccgcgttgctggcgtttttccataggctccgcccccctga
cgagcatcacaaaaatcgacgctcaagtcagaggtggcgaaacccgacaggactataaagataccaggcgtttccccctggaagctc
cctcgtgcgctctcctgttccgaccctgccgcttaccggatacctgtccgcctttctcccttcgggaagcgtggcgctttctcatagctcacgct
gtaggtatctcagttcggtgtaggtcgttcgctccaagctgggctgtgtgcacgaaccccccgttcagcccgaccgctgcgccttatccggt
aactatcgtcttgagtccaacccggtaagacacgacttatcgccactggcagcagccactggtaacaggattagcagagcgaggtatgt
aggcggtgctacagagttcttgaagtggtggcctaactacggctacactagaagaacagtatttggtatctgcgctctgctgaagccagtt
accttcggaaaaagagttggtagctcttgatccggcaaacaaaccaccgctggtagcggtggtttttttgtttgcaagcagcagattacgcg
cagaaaaaaaggatctcaagaagatcctttgatcttttctacggggtctgacgctcagtggaacgaaaactcacgttaagggattttggtc
atgagattatcaaaaaggatcttcacctagatccttttaaattaaaaatgaagttttaaatcaatctaaagtatatatgagtaaacttggtctga
cagttaccaatgcttaatcagtgaggcacctatctcagcgatctgtctatttcgttcatccatagttgcctgactccccgtcgtgtagataacta
cgatacgggagggcttaccatctggccccagtgctgcaatgataccgcgagacccacgctcaccggctccagatttatcagcaataaa
ccagccagccggaagggccgagcgcagaagtggtcctgcaactttatccgcctccatccagtctattaattgttgccgggaagctagagt
aagtagttcgccagttaatagtttgcgcaacgttgttgccattgctacaggcatcgtggtgtcacgctcgtcgtttggtatggcttcattcagctc
cggttcccaacgatcaaggcgagttacatgatcccccatgttgtgcaaaaaagcggttagctccttcggtcctccgatcgttgtcagaagta
agttggccgcagtgttatcactcatggttatggcagcactgcataattctcttactgtcatgccatccgtaagatgcttttctgtgactggtgagt
actcaaccaagtcattctgagaatagtgtatgcggcgaccgagttgctcttgcccggcgtcaatacgggataataccgcgccacatagca
gaactttaaaagtgctcatcattggaaaacgttcttcggggcgaaaactctcaaggatcttaccgctgttgagatccagttcgatgtaaccc
actcgtgcacccaactgatcttcagcatcrntactttcaccagcgtttctgggtgagcaaaaacaggaaggcaaaatgccgcaaaaaag
ggaataagggcgacacggaaatgttgaatactcatactcttcctttttcaatattattgaagcatttatcagggttattgtctcatgagcggata
catatttgaatgtatttagaaaaataaacaaataggggttccgcgcacatttccccgaaaagtgccacctgacgtctaagaaaccattatt
atcatgacattaacctataaaaataggcgtatcacgaggccctttcgtctcgcgcgtttcggtgatgacggtgaaaacctctgacacatgc
agctcccggagacggtcacagcttgtctgtaagcggatgccgggagcagacaagcccgtcagggcgcgtcagcgggtgttggcgggt
gtcggggctggcttaactatgcggcatcagagcagattgtactgagagtgcaccattcgacgctctcccttatgcgactcctgcattagga
agcagcccagtagtaggttgaggccgttgagcaccgccgccgcaaggaatggtgcatgcaaggagatggcgcccaacagtcccccg
gccacggggcctgccaccatacccacgccgaaacaagcgctcatgagcccgaagtggcgagcccgatcttccccatcggtgatgtcg
gcgatataggcgccagcaaccgcacctgtggcgccggtgatgccggccacgatgcgtccggcgtagaggatctggctagcgatgacc
ctgctgattggttcgctgaccatttccgggtgcgggacggcgttaccagaaactcagaaggttcgtccaaccaaaccgactctgacggca
grnacgagagagatgatagggtctgcttcagtaagccagatgctacacaattaggcttgtacatattgtcgttagaacgcggctacaatta
atacataaccttatgtatcatacacatacgatttaggtgacactatagaatacacggaattaattc
Bold = ITR sequence
Italics = Mpz promoter
Underline = Cx32
Italic underline = WPRE sequence
SEQ ID NO. 3: AAV-miniMpz.Egfp construct
tagctgcgcgctcgctcgctcactgaggccgcccgggcaaagcccgggcgtcgggcgacctttggtcgcccggcctcagt
gagcgagcgagcgcgcagagagggagtggccaactccatcactaggggttccttgtagttaatgattaacccgccatgcta
cttatctacgtagccatgctctaggtaccgctctcaggcaaggaggctaagaaggaatcctttgctgtagccttttggatttaggtttctca
gcttatctatccctcagagaagtgtgtctatgtcccttttctgtccctctgcctcaccccaccccaacattccaacctagggtagggggaggtc
agtatacacaaagccctctgtgtaaggggtggtatgtgtccccccacccccctacccagagtatacaatgccccttctgctccatgcccct
gccaccctcccaccacctctcaattgcacatgccaggctgcaattggtcactggctcaggacagccccctcatgctggggatccagggg
attttaagcaggttccagaaaacaccactcagttccttgtcccccgctctctccaccccacagacgctctgccaaccggtcgccaccatgg
tgagcaagggcgaggagctgttcaccggggtggtgcccatcctggtcgagctggacggcgacgtaaacggccacaagttcagcgtgt
ccggcgagggcgagggcgatgccacctacggcaagctgaccctgaagttcatctgcaccaccggcaagctgcccgtgccctggccc
accctcgtgaccaccctgacctacggcgtgcagtgcttcagccgctaccccgaccacatgaagcagcacgacttcttcaagtccgccat
gcccgaaggctacgtccaggagcgcaccatcttcttcaaggacgacggcaactacaagacccgcgccgaggtgaagttcgagggcg
acaccctggtgaaccgcatcgagctgaagggcatcgacttcaaggaggacggcaacatcctggggcacaagctggagtacaactac
aacagccacaacgtctatatcatggccgacaagcagaagascggcatcaaggtgaacttcaagatccgccacaacatcgaggacg
gcagcgtgcagctcgccgaccactaccagcagaacacccccatcggcgacggccccgtgctgctgcccgacaaccactacctgagc
acccagtccgccctgagcaaagaccccaacgagaagcgccatcacatggtcctgctggagttcgtgaccgccgccgggatcactctc
ggcatggacgagctgtacaagtaaagcggccctagatcaagcttatcgataatcaacctctggattacaaaatttgtgaaagattgactg
gtattcttaactatgttgctccttttacgctatgtggatacgctgctttaatgcctttgtatcatgctattgcttcccgtatggctttcattttctcctccttg
tataaatcctggttgctgtctctttatgaggagttgtggcccgttgtcaggcaacgtggcgtggtgtgcactgtgtttgctgacgcaaccccca
ctggttggggcattgccaccacctgtcagctcctttccgggactttcgctttccccctccctattgccacggcggaactcategccgcctgcct
tgcccgctgctggacaggggctcggctgttgggcactgacaattccgtggtgttgtcggggaaatcatcgtcctttccttggctgctcgcctgt
gttgccacctggattctgcgcgggacgtccttctgctacgtcccttcggccctcaatccagcggaccttccttcccgcggcctgctgccggct
ctgcggcctcttccgcgtcttcgccttcgccctcagacgagtcggatctccctttgggccgcctccccgcatcgataccgtcgactcgctgat
cagcctcgactgtgccttctagttgccagccatctgttgtttgcccctcccccgtgccttccttgaccctggaaggtgccactcccactgtccttt
cctaataaaatgaggaaattgcatcgcattgtctgagtaggtgtcattctattctggggggtggggtggggcaggacagcaagggggag
gattgggaagacaatagcaggcatgctggggatgcggtgggctctatggcttctgaggcggaaagaaccagctggggctcgactaga
gcatggctacgtagataagtagcatggcgggttaatcattaactacaaggaacccctagtgatggagttggccactccctctct
gcgcgctcgctcgctcactgaggccgggcgaccaaaggtcgcccgacgcccgggctttgcccgggcggcctcagtgagc
gagcgagcgcgcagagctttttgcaaaagcctaggcctccaaaaaagcctcctcactacttctggaatagctcagaggccgaggcg
gcctcggcctctgcataaataaaaaaaattagtcagccatggggcggagaatgggcggaactgggcggagttaggggcgggatggg
cggagttaggggcgggactatggttgctgactaattgagatgcatgctttgcatacttctgcctgctggggagcctggggactttccacacct
ggttgctgactaattgagatgcatgctttgcatacttctgcctgctggggagcctggggactttccacaccctaactgacacacattccacag
ctgcattaatgaatcggccaacgcgcggggagaggcggtttgcgtattgggcgctcttccgcttcctcgctcactgactcgctgcgctcggt
cgttcggctgcggcgagcggtatcagctcactcaaaggcggtaatacggttatccacagaatcaggggataacgcaggaaagaacat
gtgagcaaaaggccagcaaaaggccaggaaccgtaaaaaggccgcgttgctggcgtttttccataggctccgcccccctgacgagca
tcacaaaaatcgacgctcaagtcagaggtggcgaaacccgacaggactataaagataccaggcgtttccccctggaagctccctcgtg
cgctctcctgttccgaccctgccgcttaccggatacctgtccgcctttctcccttcgggaagcgtggcgctttctcatagctcacgctgtaggta
tctcagttcggtgtaggtcgttcgctccaagctgggctgtgtgcacgaaccccccgttcagcccgaccgctgcgccttatccggtaactatc
gtcttgagtccaacccggtaagacacgacttatcgccactggcagcagccactggtaacaggattagcagagcgaggtatgtaggcgg
tgctacagagttcttgaagtggtggcctaactacggctacactagaagaacagtatttggtatctgcgctctgctgaagccagttaccttcgg
aaaaagagttggtagctcttgatccggcaaacaaaccaccgctggtagcggtggtttttttgtttgcaagcagcagattacgcgcagaaa
aaaaggatctcaagaagatcctttgatcttttctacggggtctgacgctcagtggaacgaaaactcacgttaagggattttggtcatgagatt
atcaaaaaggatcttcacctagatccttttaaattaaaaatgaagttttaaatcaatctaaagtatatatgagtaaacttggtctgacagttac
caatgcttaatcagtgaggcacctatctcagcgatctgtctatttcgttcatccatagttgcctgactccccgtcgtgtagataactacgatacg
ggagggcttaccatctggccccagtgctgcaatgataccgcgagacccacgctcaccggctccagatttatcagcaataaaccagcca
gccggaagggccgagcgcagaagtggtcctgcaactttatccgcctccatccagtctattaattgttgccgggaagctagagtaagtagtt
cgccagttaatagtttgcgcaacgttgttgccattgctacaggcatcgtggtgtcacgctcgtcgtttggtatggcttcattcagctccggttccc
aacgatcaaggcgagttacatgatcccccatgttgtgcaaaaaagcggttagctccttcggtcctccgatcgttgtcagaagtaagttggc
cgcagtgttatcactcatggttatggcagcactgcataattctcttactgtcatgccatccgtaagatgcttttctgtgactggtgagtactcaac
caagtcattctgagaatagtgtatgcggcgaccgagttgctcttgcccggcgtcaatacgggataataccgcgccacatagcagaacttt
aaaagtgctcatcattggaaaacgttcttcggggcgaaaactctcaaggatcttaccgctgttgagatccagttcgatgtaacccactcgtg
cacccaactgatcttcagcatcttttactttcaccagcgtttctgggtgagcaaaaacaggaaggcaaaatgccgcaaaaaagggaata
agggcgacacggaaatgttgaatactcatactcttcctttttcaatattattgaagcatttatcagggttattgtctcatgagcggatacatatttg
aatgtatttagaaaaataaacaaataggggttccgcgcacatttccccgaaaagtgccacctgacgtctaagaaaccattattatcatgac
attaacctataaaaataggcgtatcacgaggccdttcgtctcgcgcgtttcggtgatgacggtgaaaacctctgacacatgcagctcccg
gagacggtcacagcttgtctgtaagcggatgccgggagcagacaagcccgtcagggcgcgtcagcgggtgttggcgggtgtcggggc
tggcttaactatgcggcatcagagcagattgtactgagagtgcaccattcgacgctctcccttatgcgactcctgcattaggaagcagccc
agtagtaggttgaggccgttgagcaccgccgccgcaaggaatggtgcatgcaaggagatggcgcccaacagtcccccggccacggg
gcctgccaccatacccacgccgaaacaagcgctcatgagcccgaagtggcgagcccgatcttccccatcggtgatgtcggcgatatag
gcgccagcaaccgcacctgtggcgccggtgatgccggccacgatgcgtccggcgtagaggatctggctagcgatgaccctgctgattg
gttcgctgaccatttccgggtgcgggacggcgttaccagaaactcagaaggttcgtccaaccaaaccgactctgacggcagtttacgag
agagatgatagggtctgcttcagtaagccagatgctacacaattaggcttgtacatattgtcgttagaacgcggctacaattaatacataac
cttatgtatcatacacatacgatttaggtgacactatagaatacacggaattaattc
Bold = ITR sequence
Italics = mini-Mpz promoter
Underline = EGFP
Italic underline = WPRE sequence
SEQ ID NO. 4: Mpz promoter
cctgttcagactcgtttcctgctgtaccctttcaatggccccacatcaaatcaaacacagatggcacatatctactctaaatatatgcagagc
ttcacaaacgtcatacacgtacgtgtgtcacacacgcacacacacacccttccacctctgcccttacctttgctgtcccatctagacattatc
cctcccatccccttatttcccttatcaaaatggctgctccttcaaggttccaaataacactgcttcctggacctgactcctctttcctctgaacttc
ctgtgttaagtgtattcctagtgcactgtgccttggtagttgttgagattgccctctgcttctcccttctgcctcctcatctagtgatcttgagcttgta
gaaagaactgaattaccattctaatacgagcattctcgaactctccaaatagccaccaagcaggacaataggcagtcttgatcatttaaa
ctgctgcatggcaaaaggaatcgaaggatttcttaacagaagtgggggggggggagatctgggcttcttcctggaagtttcctgatagag
aaaatcttctgcctgggtagaatctcccaggatgcagggagatggaaaaagtgttccccaaggactttgtagtctacaggttgtggagcc
atcggaacaacgagacaccctaatttgggagtgctctgaaagaaacttgcctctaggccctagggctctcaggcaaggaggctaagaa
ggaatcctttgctgtagccttttggatttaggtttctcagcttatctatccctcagagaagtgtgtctatgtcccttttctgtccctctgcctcacccc
accccaacattccaacctagggtagggggaggtcagtatacacaaagccctctgtgtaaggggtggtatgtgtccccccacccccctac
ccagagtatacaatgccccttctgctccatgcccctgccaccctcccaccacctctcaattgcacatgccaggctgcaattggtcactggct
caggacagccccctcatgctggggatccaggggattttaagcaggttccagaaaacaccactcagttccttgtcccccgctctctccacc
ccacagacgctctgcc
SEQ ID NO. 5: MiniMpz promoter
gctctcaggcaaggaggctaagaaggaatcctttgctgtagccttttggatttaggtttctcagcttatctatccctcagagaagtgtgtctatg
tcccttttctgtccctctgcctcaccccaccccaacattccaacctagggtagggggaggtcagtatacacaaagccctctgtgtaaggggt
ggtatgtgtccccccacccccctacccagagtatacaatgccccttctgctccatgcccctgccaccctcccaccacctctcaattgcacat
gccaggctgcaattggtcactggctcaggacagccccctcatgctggggatccaggggattttaagcaggttccagaaaacaccactca
gttccttgtcccccgctctctccaccccacagacgctctgcc
SEQ ID NO. 6: Connexin-32 (Cx32): GenBank: AY408135.1
atgaactggacaggtttgtacaccttgctcagtggcgtgaaccggcattctactgccattggccgagtatggctctcggtcatcttcatcttca
gaatcatggtgctggtggtggctgcagagagtgtgtggggtgatgagaaatcttccttcatctgcaacacactccagcctggctgcaacag
cgtttgctatgaccaattcttccccatctcccatgtgcggctgtggtccctgcagctcatcctagtttccaccccagctctcctcgtggccatgc
acgtggctcaccagcaacacatagagaagaaaatgctacggcttgagggccatggggaccccctacacctggaggaggtgaagag
gcacaaggtccacatctcagggacactgtggtggacctatgtcatcagcgtggtgttccggctgttgtttgaggccgtcttcatgtatgtctttt
atctgctctaccctggctatgccatggtgcggctggtcaagtgcgacgtctacccctgccccaacacagtggactgcttcgtgtcccgcccc
accgagaaaaccgtcttcaccgtcttcatgctagctgcctctggcatctgcatcatcctcaatgtggccgaggtggtgtacctcatcatccgg
gcctgtgcccgccgagcccagcgccgctccaatccaccttcccgcaagggctcgggcttcggccaccgcctctcacctgaatacaagc
agaatgagatcaacaagctgctgagtgagcaggatggctccctgaaagacatactgcgccgcagccctggcaccggggctgggctg
gctgaaaagagcgaccgctgctcggcctgctga
SEQ ID NO. 7: SH3 domain and tetratricopeptide repeats 2 (SH3TC2): GenBank: BC114486.1
atgggtggctgcttctgcatccccagggagcggagtctgacccggggcccaggtaaagaaactccttccaaggatccaa
ctgtatcgagtgagtgtatagcctcatctgaatacaaggaaaaatgttttctgccacagaacattaatccagacctgacactctccttctgtgt
aaagagccgctccaggaggtgtgtaaatggacccctacaggaagctgctcggaggcggctctgggcactggagaatgaggaccagg
aggtgcgcatgctgtttaaggacctctcagcaaggttggtcagtatccagtctcagagggcccagtttctcatcaccttcaagaccatgga
ggaaatctggaagttctccacctaccttaatttagaacatctcctctttgaccacaagtactggctcaactgcatattggtggaggatacaga
gatccaagtgtctgtagatgataaacacctggaaacaatatacctgggactcctgatacaggaaggccacttcttctgcagagccctgtg
ctccgtgactccaccagccgagaaggaaggggaatgcttgacactttgcaagaatgagttaatctcagtgaagatggcagaagctggc
tccgagttggaaggcgtgtctttggtgacaggtcagcggggcctggtactggtgtcagccttggagcctctgcctctccctttccaccagtgg
ttcctaaagaattatccaggaagctgtggcctttccaggaagagggattggacaggctcctatcagattggcagaggacgctgtaaggc
cttgacgggttatgagccaggagaaaaggatgaactgaatttctaccagggagaaagcattgagatcatcggctttgtcatacctgggctt
cagtggttcattggaaagtcgacaagttcaggacaagtgggctttgtccccaccaggaacatagatcctgattcttattccccaatgagca
ggaactctgcctttctcagtgatgaggagagatgctccctgttggccctgggaagtgataagcagactgagtgttccagcttcctccacact
cttgctcgcactgacatcacatctgtctaccggctcagtgggtttgaatccatccagaatcctccaaatgatctgagtgcatcccagcctga
aggcttcaaggaggtcaggcctggcagagcctgggaggagcatcaggccgtggggtccagacagtccagcagctctgaggactcca
gcctggaggaggagctcctctcggccacctcagacagctatcgcctgccggagcctgatgaccttgatgacccggaactgctcatggac
ctaagcactggtcaggaggaggaggctgagaacttcgcccccatattggcttttctggatcatgagggttatgctgaccactttaagagtct
ctatgacttctccttctctttcctcacttcttccttttatagcttctctgaggaggatgagtttgtggcctacctggaggcatcaagaaagtgggcc
aagaagagccacatgacctgggcccatgcccggctctgcttcctcctgggccggctgagcatcaggaaggtcaaactctctcaggcca
gggtgtacttcgaggaggccatccacattctcaatggagcatttgaggacctatccttggtggccactctgtacatcaatttggctgccatct
acctgaaacagaggctgagacataaaggctccgccctgttggaaaaggcaggtgccctgctggcctgcctgcctgaccgtgagtctagt
gccaagcatgaactcgacgtggtggcctacgtgctgcgccaggggattgtggtgggcagcagcccgctggaggccagggcctgctttct
ggccatccgcttgctcctgagcctaggccggcacgaggaggtcctgccctttgccgagcgcctgcagctcctctctggacaccctcctgc
ctctgaggctgtggccagtgttttgagttttctgtatgacaagaaatatcttccacaccttgcagtggcctctgtccagcaacatggtatccag
agtgcccaagggatgtctcttcctatttggcaggtccaccttgtcctccagaacacaaccaagctccttggctttccttccccaggctggggt
gaagtttctgccttggcctgcccaatgctcagacaggccctggctgcctgtgaggaactagcagaccggagcacccagagggccctgt
gtctcatcctttccaaagtgtacctcgagcacaggtctcctgacggtgccatccactacctgagccaggccttggtgctagggcagctgctg
ggtgagcaggaatcctttgagtcttctctctgcctggcatgggcctatctcttagccagccaggccaagaaggctttggatgtgcttgagcc
actgctatgctccctgaaggagacagagagtctcactcaaaggggagtcatctataacctcctgggacttgcactccaaggtgaaggcc
gggtgaacagggcagccaagagctatcttcgggccttgaacagagcccaggaggtgggagatgtgcataaccaggcagtggctatg
gccaatcttggccacctgagccttaagtcctgggctcagcatccagccagaaactatctcctgcaggctgtacgactctattgtgaacttca
ggccagtaaggagacagacatggaattagtacaggtgtttctctggttggcccaagttctggtgtctggacaccagctgacccatggcctt
ctttgttatgaaatggcattgctgtttggcttaaggcatcgacatctaaagagtcagcttcaggccaccaaatccctctgccatttctacagctc
tgtgtccccaaaccctgaggcatgcatcacctaccatgagcactggctggccctggctcagcaactcagggaccgggagatggaagg
gaggctgctggagtccctggggcagctttatcggaacctaaataccgccaggtccctcaggaggtcactcacatgcatcaaggagagc
ctgcgtatcttcattgacctgggggagacagacaaggctgctgaggcctggcttggggcggggcgactccactacctcatgcaggaag
acgagctggtggagctgtgcctgcaggcagccatccagacagccctgaagtcagaggagcctttgctggctctcaaactttatgaagaa
gcaggtgatgtgttcttcaatgggacccgccacaggcatcatgcagtggagtactaccgagctggagctgttcctttagcaaggaggttga
aggcggtgagaactgagctccggattttcaataagctgacagagctgcagattagcctcgaaggctatgagaaggctttggaatttgcca
ccctggccgccaggctcagcacagtcacaggagatcagaggcaagagctggtggcctttcaccgcctggctacagtgtactactccctg
cacatgtatgagatggctgaggactgctacctgaagaccctgtccctctgtccaccctggctgcagagtcccaaggaggccctgtactat
gccaaggtgtattatcgcctgggcagactcaccttctgccagctgaaggatgcccatgatgccactgagtacttccttctggccctggcag
cagcggtcctgctgggtgatgaggagcttcaggacaccattaggagcaggctggacaacatctgccagagccccctgtggcacagca
ggccctccgggtgctcctcagagagggcgcggtggctgagtggtggtggcctggccctctga
SEQ ID NO. 8: Peripheral myelin protein 22 (PMP22): NCBI Reference Sequence:
NM_000304.4
agttacagggagcaccaccagggaacatctcggggagcctggttggaagctgcaggcttagtctgtcggctgcgggtctctgactgccc
tgtggggagggtcttgccttaacatcccttgcatttggctgcaaagaaatctgcttggaagaaggggttacgctgtttggccgggcagaaa
ctccgctgagcagaacttgccgccagaatgctcctcctgttgctgagtatcatcgtcctccacgtcgcggtgctggtgctgctgttcgtctcca
cgatcgtcagccaatggatcgtgggcaatggacacgcaactgatctctggcagaactgtagcacctcttcctcaggaaatgtccaccact
gtttctcatcatcaccaaacgaatggctgcagtctgtccaggccaccatgatcctgtcgatcatcttcagcattctgtctctgttcctgttcttctg
ccaactcttcaccctcaccaaggggggcaggttttacatcactggaatcttccaaattcttgctggtctgtgcgtgatgagtgctgcggccat
ctacacggtgaggcacccggagtggcatctcaactcggattactcctacggtttcgcctacatcctggcctgggtggccttccccctggcc
cttctcagcggtgtcatctatgtgatcttgcggaaacgcgaatgaggcgcccagacggtctgtctgaggctctgagcgtacatagggaag
ggaggaagggaaaacagaaagcagacaaagaaaaaagagctagcccaaaatcccaaactcaaaccaaaccaaacagaaagc
agtggaggtgggggttgctgttgattgaagatgtatataatatctccggtttataaaacctatttataacactttttacatatatgtacatagtattg
tttgctttttatgttgaccatcagcctcgtgttgagccttaaagaagtagctaaggaactttacatcctaacagtataatccagctcagtatttttg
ttttgttttttgtttgtttgttttgttttacccagaaataagataactccatctcgccccttccctttcatctgaaagaagatacctccctcccagtcca
cctcatttagaaaaccaaagtgtgggtagaaaccccaaatgtccaaaagcccttttctggtgggtgacccagtgcatccaacagaaaca
gccgctgcccgaacctctgtgtgaagctttacgcgcacacggacaaaatgcccaaactggagcccttgcaaaaacacggcttgtggca
ttggcatacttgcccttacaggtggagtatcttcgtcacacatctaaatgagaaatcagtgacaacaagtctttgaaatggtgctatggattta
ccattccttattatcactaatcatctaaacaactcactggaaatccaattaacaattttacaacataagatagaatggagacctgaataattct
gtgtaatataaatggtttataactgcttttgtacctagctaggctgctattattactataatgagtaaatcataaagccttcatcactcccacatttt
tcttacggtcggagcatcagaacaagcgtctagactccttgggaccgtgagttcctagagcttggctgggtctaggctgttctgtgcctcca
aggactgtctggcaatgacttgtattggccaccaactgtagatgtatatatggtgcccttctgatgctaagactccagaccttttgtttttgctttg
cattttctgattttataccaactgtgtggactaagatgcattaaaataaacatcagagtaactca
SEQ ID NO. 9: Myelin Protein Zero (MPZ): GenBank: AK313555.1
agttcctggtcccccactttctcaaccccacagatgctccgggcccctgcccctgccccagctatggctcctggggctccctcatccagcc
ccagccctatcctggctgtgctgctcttctcttctttggtgctgtccccggcccaggccatcgtggtttacaccgacagggaggtccatggtgc
tgtgggctcccgggtgaccctgcactgctccttctggtccagtgagtgggtctcagatgacatctccttcacctggcgctaccagcccgaag
ggggcagagatgccatttcgatcttccactatgccaagggacaaccctacattgacgaggtggggaccttcaaagagcgcatccagtg
ggtaggggaccctcgctggaaggatggctccattgtcatacacaacctagactacagtgacaatggcacgttcacttgtgacgtcaaaa
accctccagacatagtgggcaagacctctcaggtcacgctgtatgtctttgaaaaagtgccaactaggtacggggtcgttctgggagctgt
gatcgggggtgtcctcggggtggtgctgttgctgctgctgcttttctacgtggttcggtactgctggctacgcaggcaggcggccctgcaga
ggaggctcagtgctatggagaaggggaaattgcacaagccaggaaaggacgcgtcgaagcgcgggcggcagacgccagtgctgt
atgcaatgctggaccacagcagaagcaccaaagctgtcagtgagaagaaggccaaggggctgggggagtctcgcaaggataaga
aatag
SEQ ID NO. 10: Early Growth Response 2 (EGR2): NCBI Reference Sequence: NM_000399.5
aactgagcgaggagcaattgattaatagctcggcgaggggactcactgactgttataataacactacaccagcaactcctggcttccca
gcagccggaacacagacaggagagagtcagtggcaaatagacatttttcttatttcttaaaaaacagcaacttgtttgctacttttatttctgtt
gatttttttttcttggtgtgtgtggtggttgtttttaagtgtggagggcaaaaggagataccatcccaggctcagtccaacccctctccaaaacg
gcttttctgacactccaggtagcgagggagttgggtctccaggttgtgcgaggagcaaatgatgaccgccaaggccgtagacaaaatcc
cagtaactctcagtggttttgtgcaccagctgtctgacaacatctacccggtggaggacctcgccgccacgtcggtgaccatctttcccaat
gccgaactgggaggcccctttgaccagatgaacggagtggccggagatggcatgatcaacattgacatgactggagagaagaggtc
gttggatctcccatatcccagcagctttgctcccgtctctgcacctagaaaccagaccttcacttacatgggcaagttctccattgaccctca
gtaccctggtgccagctgctacccagaaggcataatcaatattgtgagtgcaggcatcttgcaaggggtcacttccccagcttcaaccac
agcctcatccagcgtcacctctgcctcccccaacccactggccacaggacccctgggtgtgtgcaccatgtcccagacccagcctgacc
tggaccacctgtactctccgccaccgcctcctcctccttattctggctgtgcaggagacctctaccaggacccttctgcgttcctgtcagcag
ccaccacctccacctcttcctctctggcctacccaccacctccttcctatccatcccccaagccagccacggacccaggtctcttcccaatg
atcccagactatcctggattctttccatctcagtgccagagagacctacatggtacagctggcccagaccgtaagccctttccctgcccact
ggacaccctgcgggtgccccctccactcactccactctctacaatccgtaactttaccctggggggccccagtgctggggtgaccggacc
aggggccagtggaggcagcgagggaccccggctgcctggtagcagctcagcagcagcagcagccgccgccgccgccgcctataa
cccacaccacctgccactgcggcccattctgaggcctcgcaagtaccccaacagacccagcaagacgccggtgcacgagaggccct
acccgtgcccagcagaaggctgcgaccggcggttctcccgctctgacgagctgacacggcacatccgaatccacactgggcataagc
ccttccagtgtcggatctgcatgcgcaacttcagccgcagtgaccacctcaccacccatatccgcacccacaccggtgagaagcccttc
gcctgtgactactgtggccgaaagtttgcccggagtgatgagaggaagcgccacaccaagatccacctgagacagaaagagcggaa
aagcagtgccccctctgcatcggtgccagccccctctacagcctcctgctctgggggcgtgcagcctgggggtaccctgtgcagcagta
acagcagcagtcttggcggagggccgctcgccccttgctcctctcggacccggacaccttgagatgagactcaggctgatacaccagct
cccaaaggtcccggaggccctttgtccactggagctgcacaacaaacactaccaccctttcctgtccctctctccctttgttgggcaaagg
gctttggtggagctagcactgccccdttccacctagaagcaggttcttcctaaaacttagcccattctagtctctcttaggtgagttgactatc
aacccaaggcaaaggggaggctcagaaggaggtggtgtggggacccctggccaagagggctgaggtctgaccctgctttaaagggt
tgtttgactaggttttgctaccccacttccccttattttgacccatcacaggtttttgaccctggatgtcagagttgatctaagacgttttctacaat
aggttgggagatgctgatcccttcaagtggggacagcaaaaagacaagcaaaactgatgtgcactttatggcttgggactgatttgggg
gacattgtacagtgagtgaagtatagcctttatgccacactctgtggccctaaaatggtgaatcagagcatatctagttgtctcaacccttga
agcaatatgtattataaactcagagaacagaagtgcaatgtgatgggaggaacatagcaatatctgctccttttcgagttgtttgagaaatg
taggctattttttcagtgtatatccactcagattttgtgtatttttgatgtacactgttctctaaattctgaatctttgggaaaaaatgtaaagcatttat
gatctcagaggttaacttatttaagggggatgtacatatattctctgaaactaggatgcatgcaattgtgttggaagtgtccttggtgccttgtgt
gatgtagacaatgttacaaggtctgcatgtaaatgggttgccttattatggagaaaaaaaatcactccctgagtttagtatggctgtatatttct
gcctattaatatttggaattttttttagaaagtatatttttgtatgctttgttttgtgacttaaaagtgttacctttgtagtcaaatttcagataagaatgt
acataatgttaccggagctgatttgtttggtcattagctcttaatagttgtgaaaaaataaatctattctaacgcaaaaccactaactgaagttc
agataatggatggtttgtgactatagtgtaaataaatacttttcaacaata
SEQ ID NO. 11: Ganglioside induced differentiation associated protein 1 (GDAP1): NCBI
Reference Sequence: NM_018972.3
atggctgagaggcaggaagagcagagagggagcccgcccttgagggcggaaggcaaggccgacgcggaggttaagctcattctgt
accattggacgcattccttcagctctcaaaaggtgcgcttggtaattgctgaaaaggcattgaagtgcgaggaacatgatgtaagtctgcc
cttgagtgagcacaatgagccttggtttatgcgtttgaactcaactggagaagtgcctgtccttatccacggggaaaacataatttgtgagg
ccactcagatcattgattatcttgaacagactttcctggatgaaagaacacccaggttaatgcctgataaagaaagcatgtattacccacg
ggtacaacattaccgagagctgcttgactccttgccaatggatgcctatacacatggctgcattttacatcctgagttaactgtggactccat
gatcccggcttatgcaactacaaggattcgtagccaaattggaaacacagagtctgagctgaagaaacttgctgaagaaaacccagat
ttacaagaagcatacattgcaaaacagaaacgacttaaatcaaagctgcttgatcatgacaatgtcaagtatttgaagaaaattcttgatg
agttggagaaagtcttggatcaggttgaaactgaattgcaaagaagaaatgaagaaaccccagaagagggccagcaaccttggctct
gcggtgaatccttcaccctggcagacgtctcactcgctgtcacattgcatcgactgaagttcctggggtttgcaaggagaaactggggaa
acggaaagcgaccaaacttggaaacctattacgagcgtgtcttgaagagaaaaacatttaacaaggttttaggacatgtcaacaatatat
taatctctgcagtgctgccaacagcattccgggtggccaagaaaagggccccaaaagttcttggcacgacccttgtggttggtttgcttgc
aggagtgggatattttgcttttatgcttttcagaaagaggcttggcagcatgatattagcatttagacccagaccaaattatttctag
SEQ ID NO. 12: N-Myc downstream regulated 1 (NDRG1): NCBI Reference Sequence:
NM_001135242.1
atgtctcgggagatgcaggatgtagacctcgctgaggtgaagcctttggtggagaaaggggagaccatcaccggcctcctgcaagagt
ttgatgtccaggagcaggacatcgagactttacatggctctgttcacgtcacgctgtgtgggactcccaagggaaaccggcctgtcatcct
cacctaccatgacatcggcatgaaccacaaaacctgctacaaccccctcttcaactacgaggacatgcaggagatcacccagcacttt
gccgtctgccacgtggacgcccctggccagcaggacggcgcagcctccttccccgcagggtacatgtacccctccatggatcagctgg
ctgaaatgcttcctggagtccttcaacagtttgggctgaaaagcattattggcatgggaacaggagcaggcgcctacatcctaactcgattt
gctctaaacaaccctgagatggtggagggccttgtccttatcaacgtgaacccttgtgcggaaggctggatggactgggccgcctccaa
gatctcaggatggacccaagctctgccggacatggtggtgtcccacctttttgggaaggaagaaatgcagagtaacgtggaagtggtcc
acacctaccgccagcacattgtgaatgacatgaaccccggcaacctgcacctgttcatcaatgcctacaacagccggcgcgacctgga
gattgagcgaccaatgccgggaacccacacagtcaccctgcagtgccctgctctgttggtggttggggacagctcgcctgcagtggatg
ccgtggtggagtgcaactcaaaattggacccaacaaagaccactctcctcaagatggcggactgtggcggcctcccgcagatctccca
gccggccaagctcgctgaggccttcaagtacttcgtgcagggcatgggatacatgccctcggctagcatgacccgcctgatgcggtccc
gcacagcctctggttccagcgtcacttctctggatggcacccgcagccgctcccacaccagcgagggcacccgaagccgctcccaca
ccagcgagggcacccgcagccgctcgcacaccagcgagggggcccacctggacatcacccccaactcgggtgctgctgggaaca
gcgccgggcccaagtccatggaggtctcctgctag
SEQ ID NO. 17: AAV-human-Mpz-GJB1 construct
tagctgcgcgctcgctcgctcactgaggccgcccgggcaaagcccgggcgtcgggcgacattggtcgcccggcctcagt
gagcgagcgagcgcgcagagagggagtggccaactccatcactaggggttccttgtagttaatgattaacccgccatgcta
cttatctacgtagccatgctctaggtaccgcctggcataaacttcatttattaaagtttattttgtctttaatctctcatataacttagtcttcctga
tattgcagctgtgtgtgcccctcttttgtactcccagcattttgttcattactaaaggaagtgtcatggcttattatacttgattgttgatgggtttgtc
ctctgatcttcccatctccacctccccaaaccaaattttcaactccttgctggaaggacttaatttttattcctctctctattacctgcattctcatac
tttacatattgctggcacttaatacaattttgtagccttgaaataaattgaaatggacttaaacagcagcatgaagcactgaaggacttcttga
caaacggaaaggtcaggggcttcttgcctggaaatagtccagtggagaaaaacttctgtctgggaagaatcgcacaggatgaaggga
ggtgcggggaaaaaaactcccataggacttggtcatctcaagaagtctgtaatgcagcccacattagaggagataacaggggatatcc
tattttcagagttctctgggggaaacctccctctagttcctagggctgtgaggcagcctctctcaggcaaggaggctgaggagaaatccctt
tttatggcattaaattgaggttccatatctatccctcagagaagtgtgtctgtgtccctgtttttgtccctctccctcaccaccccccacaacattc
cagcctggggcagggggaggccagtggacacaaagccctctgtgtatggggtggtatgtgtccccccacccctccacccagactatac
aatgccccttctgctccctgcactctgcccccctccccaccacctctcaactgcacatgccaggctgcaattggttactggctgaggacag
ccccctcatgctggggccctaggggattttaagcaggttccaggaaccccccgttcagttcctggtcccccactttctcaaccccacagatg
ctccgggcccctgcccctgccccagcaccggtcgcggatcctgaggcaggatgaactggacaggtttgtacaccttgctcagtggcgtg
aaccggcattctactgccattggccgagtatggctctcggtcatcttcatcttcagaatcatggtgctggtggtggctgcagagagtgtgtgg
ggtgatgagaaatcttccttcatctgcaacacactccagcctggctgcaacagcgtttgctatgaccaattcttccccatctcccatgtgcgg
ctgtggtccctgcagctcatcctagtttccaccccagctctcctcgtggccatgcacgtggctcaccagcaacacatagagaagaaaatg
ctacggcttgagggccatggggaccccctacacctggaggaggtgaagaggcacaaggtccacatctcagggacactgtggtggac
ctatgtcatcagcgtggtgttccggctgttgtttgaggccgtcttcatgtatgtcttttatctgctctaccctggctatgccatggtgcggctggtca
agtgcgacgtctacccctgccccaacacagtggactgcttcgtgtcccgccccaccgagaaaaccgtcttcaccgtcttcatgctagctgc
ctctggcatctgcatcatcctcaatgtggccgaggtggtgtacctcatcatccgggcctgtgcccgccgagcccagcgccgctccaatcca
ccttcccgcaagggctcgggcttcggccaccgcctctcacctgaatacaagcagaatgagatcaacaagctgctgagtgagcaggatg
gctccctgaaagacatactgcgccgcagccctggcaccggggctgggctggctgaaaagagcgaccgctgctcggcctgctgactcg
agatcgatatccatcacactggcggccgcaagcttatcgataatcaacctctggattacaaaatttgtgaaagattgactggtattcttaact
atgttgctccttttacgctatgtggatacgctgctttaatgcctttgtatcatgctattgcttcccgtatggctttcattttctcctccttgtataaatcctg
gttgctgtctctttatgaggagttgtggcccgttgtcaggcaacgtggcgtggtgtgcactgtgtttgctgacgcaacccccactggttggggc
attgccaccacctgtcagctcctttccgggactttcgctttccccctccctattgccacggcggaactcatcgccgcctgccttgcccgctgct
ggacaggggctcggctgttgggcactgacaattccgtggtgttgtcggggaaatcatcgtcctttccttggctgctcgcctgtgttgccacctg
gattctgcgcgggacgtccttctgctacgtcccttcggccctcaatccagcggaccttccttcccgcggcctgctgccggctctgcggcctct
tccgcgtcttcgccttcgccctcagacgagtcggatctccctttgggccgcctccccgcatcgataccgtcgactcgctgatcagcctcgac
tgtgccttctagttgccagccatctgttgtttgcccctcccccgtgccttccttgaccctggaaggtgccactcccactgtcctttcctaataaaat
gaggaaattgcatcgcattgtctgagtaggtgtcattctattctggggggtggggtggggcaggacagcaagggggaggattgggaaga
caatagcaggcatgctggggatgcggtgggctctatggcttctgaggcggaaagaaccagctggggctcgactagagcatggctacg
tagataagtagcatggcgggttaatcattaactacaaggaacccctagtgatggagttggccactccctctctgcgcgctcgct
cgctcactgaggccgggcgaccaaaggtcgcccgacgcccgggctttgcccgggcggcctcagtgagcgagcgagcgc
gcagagctttttgcaaaagcctaggcctccaaaaaagcctcctcactacttctggaatagctcagaggccgaggcggcctcggcctctg
cataaataaaaaaaattagtcagccatggggcggagaatgggcggaactgggcggagttaggggcgggatgggcggagttagggg
cgggactatggttgctgactaattgagatgcatgctttgcatacttctgcctgctggggagcctggggactttccacacctggttgctgactaa
ttgagatgcatgctttgcatacttctgcctgctggggagcctggggactttccacaccctaactgacacacattccacagctgcattaatgaa
tcggccaacgcgcggggagaggcggtttgcgtattgggcgctcttccgcttcctcgctcactgactcgctgcgctcggtcgttcggctgcgg
cgagcggtatcagctcactcaaaggcggtaatacggttatccacagaatcaggggataacgcaggaaagaacatgtgagcaaaagg
ccagcaaaaggccaggaaccgtaaaaaggccgcgttgctggcgtttttccataggctccgcccccctgacgagcatcacaaaaatcg
acgctcaagtcagaggtggcgaaacccgacaggactataaagataccaggcgtttccccctggaagctccctcgtgcgctctcctgttcc
gaccctgccgcttaccggatacctgtccgcctttctcccttcgggaagcgtggcgctttctcatagctcacgctgtaggtatctcagttcggtgt
aggtcgttcgctccaagctgggctgtgtgcacgaaccccccgttcagcccgaccgctgcgccttatccggtaactatcgtcttgagtccaa
cccggtaagacacgacttatcgccactggcagcagccactggtaacaggattagcagagcgaggtatgtaggcggtgctacagagttc
ttgaagtggtggcctaactacggctacactagaagaacagtatttggtatctgcgctctgctgaagccagttaccttcggaaaaagagttg
gtagctcttgatccggcaaacaaaccaccgctggtagcggtggtttttttgtttgcaagcagcagattacgcgcagaaaaaaaggatctca
agaagatcdttgatcttttctacggggtctgacgctcagtggaacgaaaactcacgttaagggattttggtcatgagattatcaaaaaggat
cttcacctagatcdtttaaattaaaaatgaagttttaaatcaatctaaagtatatatgagtaaacttggtctgacagttaccaatgcttaatcag
tgaggcacctatctcagcgatctgtctatttcgttcatccatagttgcctgactccccgtcgtgtagataactacgatacgggagggcttacca
tctggccccagtgctgcaatgataccgcgagacccacgctcaccggctccagatttatcagcaataaaccagccagccggaagggcc
gagcgcagaagtggtcctgcaactttatccgcctccatccagtctattaattgttgccgggaagctagagtaagtagttcgccagttaatagt
ttgcgcaacgttgttgccattgctacaggcatcgtggtgtcacgctcgtcgtttggtatggcttcattcagctccggttcccaacgatcaaggc
gagttacatgatcccccatgttgtgcaaaaaagcggttagctccttcggtcctccgatcgttgtcagaagtaagttggccgcagtgttatcac
tcatggttatggcagcactgcataattctcttactgtcatgccatccgtaagatgcttttctgtgactggtgagtactcaaccaagtcattctgag
aatagtgtatgcggcgaccgagttgctcttgcccggcgtcaatacgggataataccgcgccacatagcagaactttaaaagtgctcatca
ttggaaaacgttcttcggggcgaaaactctcaaggatcttaccgctgttgagatccagttcgatgtaacccactcgtgcacccaactgatctt
cagcatcttttactttcaccagcgtttctgggtgagcaaaaacaggaaggcaaaatgccgcaaaaaagggaataagggcgacacgga
aatgttgaatactcatactcttcctttttcaatattattgaagcatttatcagggttattgtctcatgagcggatacatatttgaatgtatttagaaaa
ataaacaaataggggttccgcgcacatttccccgaaaagtgccacctgacgtctaagaaaccattattatcatgacattaacctataaaa
ataggcgtatcacgaggccctttcgtctcgcgcgtttcggtgatgacggtgaaaacctctgacacatgcagctcccggagacggtcacag
cttgtctgtaagcggatgccgggagcagacaagcccgtcagggcgcgtcagcgggtgttggcgggtgtcggggctggcttaactatgcg
gcatcagagcagattgtactgagagtgcaccattcgacgctctcccttatgcgactcctgcattaggaagcagcccagtagtaggttgag
gccgttgagcaccgccgccgcaaggaatggtgcatgcaaggagatggcgcccaacagtcccccggccacggggcctgccaccata
cccacgccgaaacaagcgctcatgagcccgaagtggcgagcccgatcttccccatcggtgatgtcggcgatataggcgccagcaacc
gcacctgtggcgccggtgatgccggccacgatgcgtccggcgtagaggatctggctagcgatgaccctgctgattggttcgctgaccattt
ccgggtgcgggacggcgttaccagaaactcagaaggttcgtccaaccaaaccgactctgacggcagtttacgagagagatgataggg
tctgcttcagtaagccagatgctacacaattaggcttgtacatattgtcgttagaacgcggctacaattaatacataaccttatgtatcataca
catacgatttaggtgacactatagaatacacggaattaattc
Bold = ITR sequence
Italics = human Mpz promoter
Underline = Cx32
Italic underline = WPRE sequence
SEQ ID NO. 18: Human hP0 promoter
gcctggcataaacttcatttattaaagtttattttgtctttaatctctcatataacttagtcttcctgatattgcagctgtgtgtgcccctcttttgtactc
ccagcattttgttcattactaaaggaagtgtcatggcttattatacttgattgttgatgggtttgtcctctgatcttcccatctccacctccccaaac
caaattttcaactccttgctggaaggacttaatttttattcctctctctattacctgcattctcatactttacatattgctggcacttaatacaattttgt
agccttgaaataaattgaaatggacttaaacagcagcatgaagcactgaaggacttcttgacaaacggaaaggtcaggggcttcttgcc
tggaaatagtccagtggagaaaaacttctgtctgggaagaatcgcacaggatgaagggaggtgcggggaaaaaaactcccatagga
cttggtcatctcaagaagtctgtaatgcagcccacattagaggagataacaggggatatcctattttcagagttctctgggggaaacctccc
tctagttcctagggctgtgaggcagcctctctcaggcaaggaggctgaggagaaatccctttttatggcctttaaattgaggttccatatctat
ccctcagagaagtgtgtctgtgtccctgtttttgtccctctccctcaccaccccccacaacattccagcctggggcagggggaggccagtg
gacacaaagccctctgtgtatggggtggtatgtgtccccccacccctccacccagactatacaatgccccttctgctccctgcactctgccc
ccctccccaccacctctcaactgcacatgccaggctgcaattggttactggctgaggacagccccctcatgctggggccctaggggatttt
aagcaggttccaggaaccccccgttcagttcctggtcccccactttctcaaccccacagatgctccgggcccctgcccctgccccagc
SEQ ID NO. 19: AAV-human-Mpz-Egfp mock construct
tagctgcgcgctcgctcgctcactgaggccgcccgggcaaagcccgggcgtcgggcgacctttggtcgcccggcctcagt
gagcgagcgagcgcgcagagagggagtggccaactccatcactaggggttccttgtagttaatgattaacccgccatgcta
cttatctacgtagccatgctctaggtaccgcctggcataaacttcatttattaaagtttattttgtctttaatctctcatataacttagtcttcctga
tattgcagctgtgtgtgcccctcttttgtactcccagcattttgttcattactaaaggaagtgtcatggcttattatacttgattgttgatgggtttgtc
ctctgatcttcccatctccacctccccaaaccaaattttcaactccttgctggaaggacttaatttttattcctctctctattacctgcattctcatac
tttacatattgctggcacttaatacaattttgtagccttgaaataaattgaaatggacttaaacagcagcatgaagcactgaaggacttcttga
caaacggaaaggtcaggggcttcttgcctggaaatagtccagtggagaaaaacttctgtctgggaagaatcgcacaggatgaaggga
ggtgcggggaaaaaaactcccataggacttggtcatctcaagaagtctgtaatgcagcccacattagaggagataacaggggatatcc
tattttcagagttctctgggggaaacctccctctagttcctagggctgtgaggcagcctctctcaggcaaggaggctgaggagaaatccctt
tttatggcattaaattgaggttccatatctatccctcagagaagtgtgtctgtgtccctgtttttgtccctctccctcaccaccccccacaacattc
cagcctggggcagggggaggccagtggacacaaagccctctgtgtatggggtggtatgtgtccccccacccctccacccagactatac
aatgccccttctgctccctgcactctgcccccctccccaccacctctcaactgcacatgccaggctgcaattggttactggctgaggacag
ccccctcatgctggggccctaggggattttaagcaggttccaggaaccccccgttcagttcctggtcccccactttctcaaccccacagatg
ctccgggcccctgcccctgccccagcaccggtcgccaccatggtgagcaagggcgaggagctgttcaccggggtggtgcccatcctg
gtcgagctggacggcgacgtaaacggccacaagttcagcgtgtccggcgagggcgagggcgatgccacctacggcaagctgaccct
gaagttcatctgcaccaccggcaagctgcccgtgccctggcccaccctcgtgaccaccctgacctacggcgtgcagtgcttcagccgct
accccgaccacatgaagcagcacgacttcttcaagtccgccatgcccgaaggctacgtccaggagcgcaccatcttcttcaaggacga
cggcaactacaagacccgcgccgaggtgaagttcgagggcgacaccctggtgaaccgcatcgagctgaagggcatcgacttcaagg
aggacggcaacatcctggggcacaagctggagtacaactacaacagccacaacgtctatatcatggccgacaagcagaagaacgg
catcaaggtgaacttcaagatccgccacaacatcgaggacggcagcgtgcagctcgccgaccactaccagcagaacacccccatcg
gcgacggccccgtgctgctgcccgacaaccactacctgagcacccagtccgccctgagcaaagaccccaacgagaagcgcgatca
catggtcctgctggagttcgtgaccgccgccgggatcactctcggcatggacgagctgtacaagtaaagcggccctagatcaagcttatc
gataatcaacctctggattacaaaatttgtgaaagattgactggtattcttaactatgttgctccttttacgctatgtggatacgctgctttaatgc
ctttgtatcatgctattgcttcccgtatggctttcattttctcctccttgtataaatcctggttgctgtctctttatgaggagttgtggcccgttgtcagg
caacgtggcgtggtgtgcactgtgtttgctgacgcaacccccactggttggggcattgccaccacctgtcagctcctttccgggactttcgctt
tccccctccctattgccacggcggaactcatcgccgcctgccttgcccgctgctggacaggggctcggctgttgggcactgacaattccgt
ggtgttgtcggggaaatcatcgtcctttccttggctgctcgcctgtgttgccacctggattctgcgcgggacgtccttctgctacgtcccttcggc
cctcaatccagcggaccttccttcccgcggcctgctgccggctctgcggcctcttccgcgtcttcgccttcgccctcagacgagtcggatctc
cctttgggccgcctccccgcatcgataccgtcgactcgctgatcagcctcgactgtgccttctagttgccagccatctgttgtttgcccctccc
ccgtgccttccttgaccctggaaggtgccactcccactgtcctttcctaataaaatgaggaaattgcatcgcattgtctgagtaggtgtcattct
attctggggggtggggtggggcaggacagcaagggggaggattgggaagacaatagcaggcatgctggggatgcggtgggctctat
ggcttctgaggcggaaagaaccagctggggctcgactagagcatggctacgtagataagtagcatggcgggttaatcattaacta
caaggaacccctagtgatggagttggccactccctctctgcgcgctcgctcgctcactgaggccgggcgaccaaaggtcgc
ccgacgcccgggctttgcccgggcggcctcagtgagcgagcgagcgcgcagagctttttgcaaaagcctaggcctccaaaa
aagcctcctcactacttctggaatagctcagaggccgaggcggcctcggcctctgcataaataaaaaaaattagtcagccatggggcg
gagaatgggcggaactgggcggagttaggggcgggatgggcggagttaggggcgggactatggttgctgactaattgagatgcatgct
ttgcatacttctgcctgctggggagcctggggactttccacacctggttgctgactaattgagatgcatgctttgcatacttctgcctgctgggg
agcctggggactttccacaccctaactgacacacattccacagctgcattaatgaatcggccaacgcgcggggagaggcggtttgcgta
ttgggcgctcttccgcttcctcgctcactgactcgctgcgctcggtcgttcggctgcggcgagcggtatcagctcactcaaaggcggtaata
cggttatccacagaatcaggggataacgcaggaaagaacatgtgagcaaaaggccagcaaaaggccaggaaccgtaaaaaggc
cgcgttgctggcgtttttccataggctccgcccccctgacgagcatcacaaaaatcgacgctcaagtcagaggtggcgaaacccgaca
ggactataaagataccaggcgtttccccctggaagctccctcgtgcgctctcctgttccgaccctgccgcttaccggatacctgtccgccttt
ctcccttcgggaagcgtggcgctttctcatagctcacgctgtaggtatctcagttcggtgtaggtcgttcgctccaagctgggctgtgtgcacg
aaccccccgttcagcccgaccgctgcgccttatccggtaactatcgtcttgagtccaacccggtaagacacgacttatcgccactggcag
cagccactggtaacaggattagcagagcgaggtatgtaggcggtgctacagagttcttgaagtggtggcctaactacggctacactaga
agaacagtatttggtatctgcgctctgctgaagccagttaccttcggaaaaagagttggtagctcttgatccggcaaacaaaccaccgctg
gtagcggtggtttttttgtttgcaagcagcagattacgcgcagaaaaaaaggatctcaagaagatcctttgatcttttctacggggtctgacg
ctcagtggaacgaaaactcacgttaagggattttggtcatgagattatcaaaaaggatcttcacctagatccttttaaattaaaaatgaagtt
ttaaatcaatctaaagtatatatgagtaaacttggtctgacagttaccaatgcttaatcagtgaggcacctatctcagcgatctgtctatttcgtt
catccatagttgcctgactccccgtcgtgtagataactacgatacgggagggcttaccatctggccccagtgctgcaatgataccgcgag
acccacgctcaccggctccagatttatcagcaataaaccagccagccggaagggccgagcgcagaagtggtcctgcaactttatccgc
ctccatccagtctattaattgttgccgggaagctagagtaagtagttcgccagttaatagtttgcgcaacgttgttgccattgctacaggcatc
gtggtgtcacgctcgtcgtttggtatggcttcattcagctccggttcccaacgatcaaggcgagttacatgatcccccatgttgtgcaaaaaa
gcggttagctccttcggtcctccgatcgttgtcagaagtaagttggccgcagtgttatcactcatggttatggcagcactgcataattctcttac
tgtcatgccatccgtaagatgcttttctgtgactggtgagtactcaaccaagtcattctgagaatagtgtatgcggcgaccgagttgctcttgc
ccggcgtcaatacgggataataccgcgccacatagcagaactttaaaagtgctcatcattggaaaacgttcttcggggcgaaaactctc
aaggatcttaccgctgttgagatccagttcgatgtaacccactcgtgcacccaactgatcttcagcatcttttactttcaccagcgtttctgggt
gagcaaaaacaggaaggcaaaatgccgcaaaaaagggaataagggcgacacggaaatgttgaatactcatactcttcctttttcaata
ttattgaagcatttatcagggttattgtctcatgagcggatacatatttgaatgtatttagaaaaataaacaaataggggttccgcgcacatttc
cccgaaaagtgccacctgacgtctaagaaaccattattatcatgacattaacctataaaaataggcgtatcacgaggccctttcgtctcgc
gcgtttcggtgatgacggtgaaaacctctgacacatgcagctcccggagacggtcacagcttgtctgtaagcggatgccgggagcaga
caagcccgtcagggcgcgtcagcgggtgttggcgggtgtcggggctggcttaactatgcggcatcagagcagattgtactgagagtgca
ccattcgacgctctcccttatgcgactcctgcattaggaagcagcccagtagtaggttgaggccgttgagcaccgccgccgcaaggaat
ggtgcatgcaaggagatggcgcccaacagtcccccggccacggggcctgccaccatacccacgccgaaacaagcgctcatgagcc
cgaagtggcgagcccgatcttccccatcggtgatgtcggcgatataggcgccagcaaccgcacctgtggcgccggtgatgccggccac
gatgcgtccggcgtagaggatctggctagcgatgaccctgctgattggttcgctgaccatttccgggtgcgggacggcgttaccagaaac
tcagaaggttcgtccaaccaaaccgactctgacggcagtttacgagagagatgatagggtctgcttcagtaagccagatgctacacaatt
aggcttgtacatattgtcgttagaacgcggctacaattaatacataaccttatgtatcatacacatacgatttaggtgacactatagaataca
cggaattaattc
Bold = ITR sequence
Italics = human Mpz promoter
Underline = EGFP
Italic underline = WPRE sequence
SEQ ID NO. 20: AAV-minMpz-SH3TC2.myc.ITR for therapeutic SH3TC2 gene replacement
tagctgcgcgctcgctcgctcactgaggccgcccgggcaaagcccgggcgtcgggcgacctttggtcgcccggcctcagt
gagcgagcgagcgcgcagagagggagtggccaactccatcactaggggttccttgtagttaatgattaacccgccatgcta
cttatctacgtagccatgctctaggtaccgctctcaggcaaggaggctaagaaggaatcctttgctgtagccttttggatttaggtttctca
gcttatctatccctcagagaagtgtgtctatgtcccttttctgtccctctgcctcaccccaccccaacattccaacctagggtagggggaggtc
agtatacacaaagccctctgtgtaaggggtggtatgtgtccccccacccccctacccagagtatacaatgccccttctgctccatgcccct
gccacccctcccaccacctctcaattgcacatgccaggctgcaattggtcactggctcaggacagccccctcatgctggggatccaggg
gattttaagcaggttccagaaaacaccactcagttccttgtcccccgctctctccaccccacagacgctctgccaaccggtaccatgggtg
gctgcttctgcatccccagggagcggagtctgacccgcmcccaggtaaagaaactccttccaaggatccaactgtatcgagtgagtgt
atagcctcatctgaatacaaggaaaaatgttttctgccacagaacattaatccagacctgacactctccttctgtgtaaagagccgctccag
gaggtgtgtaaatggacccctacaggaagctgctcggaggcggctctgggcactggagaatgaggaccaggaggtgcgcatgctgttt
aaggacctctcagcaaggttggtcagtatccagtctcagagggcccagtttctcatcaccttcaagaccatggaggaaatctggaagttct
ccacctaccttaatttaggctacgtatccatgtgtctagaacatctcctctttgaccacaagtactggctcaactgcatattggtggaggatac
agagatccaagtactgtagatgataaacacctggaaacaatatacctgggactcctgatacaggaaggccacttcttctgcagagccct
gtgctccgtgactccaccagccgagaaggaaggggaatgcttgacactttgcaagaatgagttaatctcagtgaagatggcagaagct
ggctccgagttggaaggcgtgtctttggtgacaggtcagcggggcctggtactggtgtcagccttggagcctctgcctctccctttccacca
gtggttcctaaagaattatccaggaagctgtggcctttccaggaagagggattggacaggctcctatcagattggcagaggacgctgtaa
ggccttgacgggttatgagccaggagaaaaggatgaactgaatttctaccagggagaaagcattgagatcatcggctttgtcatacctgg
gcttcagtggttcattggaaagtcgacaagttcaggacaagtgggctttgtccccaccaggaacatagatcctgattcttattccccaatga
gcaggaactctgcctttctcagtgatgaggagagatgctccctgttggccctgggaagtgataagcagactgagtgttccagcttcctcca
cactcttgctcgcactgacatcacatctgtctaccggctcagtgggtttgaatccatccagaatcctccaaatgatctgagtgcatcccagcc
tgaaggtttcaaggaggtcaggcctggcagagcctgggaggagcatcaggccgtggggtccagacagtccagcagctctgaggactc
cagcctggaggaggagctcctctcggccacctcagacagctatcgcctgccggagcctgatgaccttgatgacccggaactgctcatgg
acctaagcactggtcaggaggaggaggctgagaacttcgcccccatattggcttttctggatcatgagggttatgctgaccactttaagagt
ctctatgacttctccttctctttcctcacttcttcgtttatagcttctctgaggaggatgagtttgtggcctacctggaggcatcaagaaagtgggc
caagaagagccacatgacctgggcccatgcccggctctgcttcctcctgggccggctgagcatcaggaaggtcaaactctctcaggcc
agggtgtacttcgaggaggccatccacattctcaatggagcatttgaggacctatccttggtggccactctgtacatcaatttggctgccatc
tacctgaaacagaggctgagacataaaggctccgccctgttggaaaaggcaggtgccctgctggcctgcctgcctgaccgtgagtctag
tgccaagcatgaactcgacgtggtggcctacgtgctgcgccaggggattgtggtgggcagcagcccgctggaggccagggcctgctttc
tggccatccgcttgctcctgagcctaggccggcacgaggaggtcctgccctttgccgagcgcctgcagctcctctctggacaccctcctgc
ctctgaggctgtggccagtgttttgagttttctgtatgacaagaaatatcttccacaccttgcagtggcctctgtccagcaacatggtatccag
agtgcccaagggatgtctcttcctatttggcaggtccaccttgtcctccagaacacaaccaagctccttggctttccttccccaggctggggt
gaagtttctgccttggcctgcccaatgctcagacaggccctggctgcctgtgaggaactagcagaccggagcacccagagggccctgt
gtctcatcctttccaaagtgtacctcgagcacaggtctcctgacggtgccatccactacctgagccaggccttggtgctagggcagctgctg
ggtgagcaggaatcctttgagtcttctctctgcctggcatgggcctatctcttagccagccaggccaagaaggctttggatgtgcttgagcc
actgctatgctccctgaaggagacagagagtctcactcaaaggggagtcatctataacctcctgggacttgcactccaaggtgaaggcc
gggtgaacagggcagccaagagctatcttcgggccttgaacagagcccaggaggtgggagatgtgcataaccaggcagtggctatg
gccaatcttggccacctgagccttaagtcctgggctcagcatccagccagaaactatctcctgcaggctgtacgactctattgtgaacttca
ggccagtaaggagacagacatggaattagtacaggtgtttctctggttggcccaagttctggtgtctggacaccagctgacccatggcctt
ctttgttatgaaatggcattgctgtttggcttaaggcatcgacatctaaagagtcagcttcaggccaccaaatccctctgccatttctacagctc
tgtgtccccaaaccctgaggcatgcatcacctaccatgagcactggctggccctggctcagcaactcagggaccgggagatggaagg
gaggctgctggagtccctggggcagctttatcggaacctaaataccgccaggtccctcaggaggtcactcacatgcatcaaggagagc
ctgcgtatcttcattgacctgggggagacagacaaggctgctgaggcctggcttggggcggggcgactccactacctcatgcaggaag
acgagctggtggagctgtgcctgcaggcagccatccagacagccctgaagtcagaggagcctttgctggctctcaaactttatgaagaa
gcaggtgatgtgttcttcaatgggacccgccacaggcatcatgcagtggagtactaccgagctggagctgttcctttagcaaggaggttga
aggcggtgagaactgagctccggattttcaataagctgacagagctgcagattagcctcgaaggctatgagaaggctttggaatttgcca
ccctggccgccaggctcagcacagtcacaggagatcagaggcaagagctggtggcctttcaccgcctggctacagtgtactactccctg
cacatgtatgagatggctgaggactgctacctgaagaccctgtccctctgtccaccatggctgcagagtcccaaggaggccctgtactat
gccaaggtgtattatcgcctgggcagactcaccttctgccagctgaaggatgcccatgatgccactgagtacttccttctggccctggcag
cagcggtcctgctgggtgatgaggagcttcaggacaccattaggagcaggctggacaacatctgccagagccccctgtggcacagca
ggccctccgggtgctcctcagagagggcgcggtggctgagtggtggtggcctggccctcgagcagaagctgatcagcgaggaggac
ctgtaagatatccatcacactggcggccgcggagctctcgagaggcctaataaagagctcagatgcatcgatcagagtgtgttggttttttg
tgtgagatctaagcttagcatggctacgtagataagtagcatggcgggttaatcattaactacaaggaacccctagtgatggagtt
ggccactccctctctgcgcgctcgctcgctcactgaggccgggcgaccaaaggtcgcccgacgcccgggctttgcccggg
cggcctcagtgagcgagcgagcgcgcagagctttttgcaaaagcctaggcctccaaaaaagcctcctcactacttctggaatagct
cagaggccgaggcggcctcggcctctgcataaataaaaaaaattagtcagccatggggcggagaatgggcggaactgggcggagtt
aggggcgggatgggcggagttaggggcgggactatggttgctgactaattgagatgcatgctttgcatacttctgcctgctggggagcctg
gggactttccacacctggttgctgactaattgagatgcatgctttgcatacttctgcctgctggggagcctggggactttccacaccctaactg
acacacattccacagctgcattaatgaatcggccaacgcgcggggagaggcggtttgcgtattgggcgctcttccgcttcctcgctcactg
actcgctgcgctcggtcgttcggctgcggcgagcggtatcagctcactcaaaggcggtaatacggttatccacagaatcaggggataac
gcaggaaagaacatgtgagcaaaaggccagcaaaaggccaggaaccgtaaaaaggccgcgttgctggcgtttttccataggctccg
cccccctgacgagcatcacaaaaatcgacgctcaagtcagaggtggcgaaacccgacaggactataaagataccaggcgtttcccc
ctggaagctccctcgtgcgctctcctgttccgaccctgccgcttaccggatacctgtccgcctttctcccttcgggaagcgtggcgctttctcat
agctcacgctgtaggtatctcagttcggtgtaggtcgttcgctccaagctgggctgtgtgcacgaaccccccgttcagcccgaccgctgcg
ccttatccggtaactatcgtcttgagtccaacccggtaagacacgacttatcgccactggcagcagccactggtaacaggattagcagag
cgaggtatgtaggcggtgctacagagttcttgaagtggtggcctaactacggctacactagaagaacagtatttggtatctgcgctctgctg
aagccagttaccttcggaaaaagagttggtagctcttgatccggcaaacaaaccaccgctggtagcggtggtttttttgtttgcaagcagca
gattacgcgcagaaaaaaaggatctcaagaagatcctttgatcttttctacggggtctgacgctcagtggaacgaaaactcacgttaagg
gattttggtcatgagattatcaaaaaggatcttcacctagatccttttaaattaaaaatgaagttttaaatcaatctaaagtatatatgagtaaa
cttggtctgacagttaccaatgcttaatcagtgaggcacctatctcagcgatctgtctatttcgttcatccatagttgcctgactccccgtcgtgt
agataactacgatacgggagggcttaccatctggccccagtgctgcaatgataccgcgagacccacgctcaccggctccagatttatca
gcaataaaccagccagccggaagggccgagcgcagaagtggtcctgcaactttatccgcctccatccagtctattaattgttgccggga
agctagagtaagtagttcgccagttaatagtttgcgcaacgttgttgccattgctacaggcatcgtggtgtcacgctcgtcgtttggtatggctt
cattcagctccggttcccaacgatcaaggcgagttacatgatcccccatgttgtgcaaaaaagcggttagctccttcggtcctccgatcgtt
gtcagaagtaagttggccgcagtgttatcactcatggttatggcagcactgcataattctcttactgtcatgccatccgtaagatgcttttctgtg
actggtgagtactcaaccaagtcattctgagaatagtgtatgcggcgaccgagttgctcttgcccggcgtcaatacgggataataccgcg
ccacatagcagaactttaaaagtgctcatcattggaaaacgttcttcggggcgaaaactctcaaggatcttaccgctgttgagatccagttc
gatgtaacccactcgtgcacccaactgatcttcagcatcttttactttcaccagcgtttctgggtgagcaaaaacaggaaggcaaaatgcc
gcaaaaaagggaataagggcgacacggaaatgttgaatactcatactcttcctttttcaatattattgaagcatttatcagggttattgtctcat
gagcggatacatatttgaatgtatttagaaaaataaacaaataggggttccgcgcacatttccccgaaaagtgccacctgacgtctaaga
aaccattattatcatgacattaacctataaaaataggcgtatcacgaggccgttcgtctcgcgcgtttcggtgatgacggtgaaaacctctg
acacatgcagctcccggagacggtcacagcttgtctgtaagcggatgccgggagcagacaagcccgtcagggcgcgtcagcgggtgt
tggcgggtgtcggggctggcttaactatgcggcatcagagcagattgtactgagagtgcaccattcgacgctctcccttatgcgactcctgc
attaggaagcagcccagtagtaggttgaggccgttgagcaccgccgccgcaaggaatggtgcatgcaaggagatggcgcccaacag
tcccccggccacggggcctgccaccatacccacgccgaaacaagcgctcatgagcccgaagtggcgagcccgatcttccccatcggt
gatgtcggcgatataggcgccagcaaccgcacctgtggcgccggtgatgccggccacgatgcgtccggcgtagaggatctggctagc
gatgaccctgctgattggttcgctgaccatttccgggtgcgggacggcgttaccagaaactcagaaggttcgtccaaccaaaccgactct
gacggcagtttacgagagagatgatagggtctgcttcagtaagccagatgctacacaattaggcttgtacatattgtcgttagaacgcggc
tacaattaatacataaccttatgtatcatacacatacgatttaggtgacactatagaatacacggaattaattc
Bold = ITR sequence
Italics = mini Mpz promoter
Underline = SH3TC2
Italic underline = synthetic minimal polyA
SEQ ID NO. 21: AAV-human-miniMpz-SH3TC2
tagctgcgcgctcgctcgctcactgaggccgcccgggcaaagcccgggcgtcgggcgacctttggtcgcccggcctcagt
gagcgagcgagcgcgcagagagggagtggccaactccatcactaggggttccttgtagttaatgattaacccgccatgcta
cttatctacgtagccatgctctaggtacctctctcaggcaaggaggctgaggagaaatccctttttatggcctttaaattgaggttccatat
ctatccctcagagaagtgtgtctgtgtccctgtttttgtccctctccctcaccaccccccacaacattccagcctggggcagggggaggcca
gtggacacaaagccctctgtgtatggggtggtatgtgtccccccacccctccacccagactatacaatgccccttctgctccctgcactctg
cccccctccccaccacctctcaactgcacatgccaggctgcaattggttactggctgaggacagccccctcatgctggggccctagggg
attttaagcaggttccaggaaccccccgttcagttcctggtcccccactttctcaaccccacagatgctccgggcccctgcccctgccccag
cggtaccatgaataactacttctacatccccaaaaagcgaaatctaacccgaggcccaaataaaaaaactccttccaaaaatccaact
gtatcgagtgagtgtatagcctcatctgaatacaaggaaaaatgttttctgccacagaacattaatccagacctgacactctccttctgtgta
aagagccgctccaggaggtgtgtaaatggacccctacaggaagctgctcggaggcggctctgggcactggagaatgaggaccagga
ggtgcgcatgctgtttaaggacctctcagcaaggttggtcagtatccagtctcagagggcccagtttctcatcaccttcaagaccatggag
gaaatctggaagttctccacctaccttaatttaggctacgtatccatgtgtctagaacatctcctctttgaccacaagtactggctcaactgcat
attggtggaggatacagagatccaagtgtctgtagatgataaacacctggaaacaatatacctgggactcctgatacaggaaggccact
tcttctgcagagccctgtgctccgtgactccaccagccgagaaggaaggggaatgcttgacactttgcaagaatgagttaatctcagtga
agatggcagaagctggctccgagttggaaggcgtgtctttggtgacaggtcagcggggcctggtactggtgtcagccttggagcctctgc
ctctccctttccaccagtggttcctaaagaattatccaggaagctgtggcctttccaggaagagggattggacaggctcctatcagattggc
agaggacgctgtaaggccttgacgggtlatgagccaggagaaaaggatgaactgaatttctaccagggagaaagcattgagatcatc
ggctttgtcatacctgggcttcagtggttcattggaaagtcgacaagttcaggacaagtgggctttgtccccaccaggaacatagatcctga
ttcttattccccaatgagcaggaactctgcctttctcagtgatgaggagagatgctccctgttggccctgggaagtgataagcagactgagt
gttccagcttcctccacactcttgctcgcactgacatcacatctgtctaccggctcagtgggtttgaatccatccagaatcctccaaatgatct
gagtgcatcccagcctgaaggtttcaaggaggtcaggcctggcagagcctgggaggagcatcaggccgtggggtccagacagtcca
gcagctctgaggactccagcctggaggaggagctcctctcggccacctcagacagctatcgcctgccggagcctgatgaccttgatgac
ccggaactgctcatggacctaagcactggtcaggaggaggaggctgagaacttcgcccccatattggcttttctggatcatgagggttatg
ctgaccactttaagagtctctatgacttctccttctctttcctcacttcttccttttatagcttctctgaggaggatgagtttgtggcctacctggagg
catcaagaaagtgggccaagaagagccacatgacctgggcccatgcccggctctgcttcctcctgggccggctgagcatcaggaagg
tcaaactctctcaggccagggtgtacttcgaggaggccatccacattctcaatggagcatttgaggacctatccttggtggccactctgtac
atcaatttggctgccatctacctgaaacagaggctgagacataaaggctccgccctgttggaaaaggcaggtgccctgctggcctgcctg
cctgaccgtgagtctagtgccaagcatgaactcgacgtggtggcctacgtgctgcgccaggggattgtggtgggcagcagcccgctgg
aggccagggcctgctttctggccatccgcttgctcctgagcctaggccggcacgaggaggtcctgccctttgccgagcgcctgcagctcc
tctctggacaccctcctgcctctgaggctgtggccagtgttttgagttttctgtatgacaagaaatatcttccacaccttgcagtggcctctgtcc
agcaacatggtatccagagtgcccaagggatgtctcttcctatttggcaggtccaccttgtcctccagaacacaaccaagctccttggcttt
ccttccccaggctggggtgaagtttctgccttggcctgcccaatgctcagacaggccctggctgcctgtgaggaactagcagaccggag
cacccagagggccctgtgtctcatcctttccaaagtgtacctcgagcacaggtctcctgacggtgccatccactacctgagccaggccttg
gtgctagggcagctgctgggtgagcaggaatcctttgagtcttctctctgcctggcatgggcctatctcttagccagccaggccaagaagg
ctttggatgtgcttgagccactgctatgctccctgaaggagacagagagtctcactcaaaggggagtcatctataacctcctgggacttgc
actccaaggtgaaggccgggtgaacagggcagccaagagctatcttcgggccttgaacagagcccaggaggtgggagatgtgcata
accaaacaataactatagccaatcttagccacctaaaccttaaatcctagactcaacatccagccaaaaactatctcctacaaactgtac
gactctattgtgaacttcaggccagtaaggagacagacatggaattagtacaggtgtttctctggttggcccaagttctggtgtctggacac
cagctgacccatggccttctttgttatgaaatggcattgctgttlggcttaaggcatcgacatctaaagagtcagcttcaggccaccaaatcc
ctctgccatttctacagctctgtgtccccaaaccctgaggcatgcatcacctaccatgagcactggctggccctggctcagcaactcaggg
accgggagatggaagggaggctgctggagtccctggggcagctttatcggaacctaaataccgccaggtccctcaggaggtcactca
catgcatcaaggagagcctgcgtatcttcattgacctgggggagacagacaaggctgctgaggcctggcttggggcggggcgactcca
ctacctcatgcaggaagacgagctggtggagctgtgcctgcaggcagccatccagacagccctgaagtcagaggagcctttgctggct
ctcaaactttatgaagaagcaggtgatgtgttcttcaatgggacccgccacaggcatcatgcagtggagtactaccgagctggagctgttc
ctttagcaaggaggttgaaggcggtgagaactgagctccggattttcaataagctgacagagctgcagattagcctcgaaggctatgag
aaggctttggaatttgccaccctggccgccaggctcagcacagtcacaggagatcagaggcaagagctggtggcctttcaccgcctgg
ctacagtgtactactccctgcacatgtatgagatggctgaggactgctacctgaagaccctgtccctctgtccaccatggctgcagagtccc
aaggaggccctgtactatgccaaggtgtattatcgcctgggcagactcaccttctgccagctgaaggatgcccatgatgccactgagtact
tccttctggccctggcagcagcggtcctgctgggtgatgaggagcttcaggacaccattaggagcaggctggacaacatctgccagagc
cccctgtggcacagcaggccctccgggtgctcctcagagagggcgcggtggctgagtggtggtggcctggccctctgagcggccgcg
gagctctcgagaggcctaataaagagctcagatgcatcgatcagagtgtgttggttttttgtgtgagatctaagcttagcatggctacgta
gataagtagcatggcgggttaatcattaactacaaggaacccctagtgatggagttggccactccctctctgcgcgctcgctc
gctcactgaggccgggcgaccaaaggtcgcccgacgcccgggctttgcccgggcggcctcagtgagcgagcgagcgcg
cagagctttttgcaaaagcctaggcctccaaaaaagcctcctcactacttctggaatagctcagaggccgaggcggcctcggcctctgc
ataaataaaaaaaattagtcagccatggggcggagaatgggcggaactgggcggagttaggggcgggatgggcggagttaggggc
gggactatggttgctgactaattgagatgcatgctttgcatacttctgcctgctggggagcctggggactttccacacctggttgctgactaatt
gagatgcatgctttgcatacttctgcctgctggggagcctggggactttccacaccctaactgacacacattccacagctgcattaatgaat
cggccaacgcgcggggagaggcggtttgcgtattgggcgctcttccgcttcctcgctcactgactcgctgcgctcggtcgttcggctgcgg
cgagcggtatcagctcactcaaaggcggtaatacggttatccacagaalcaggggataacgcaggaaagaacatgtgagcaaaagg
ccagcaaaaggccaggaaccgtaaaaaggccgcgttgctggcgtttttccataggctccgcccccctgacgagcatcacaaaaatcg
acgctcaagtcagaggtggcgaaacccgacaggactataaagataccaggcgtttccccctggaagctccctcgtgcgctctcctgttcc
gaccctgccgcttaccggatacctgtccgcctttctcccttcgggaagcgtggcgctttctcatagctcacgctgtaggtatctcagttcggtgt
aggtcgttcgctccaagctgggctgtgtgcacgaaccccccgttcagcccgaccgctgcgccttatccggtaactatcgtcttgagtccaa
cccggtaagacacgacttatcgccactggcagcagccactggtaacaggattagcagagcgaggtatgtaggcggtgctacagagttc
ttgaagtggtggcctaactacggctacactagaagaacagtatttggtatctgcgctctgctgaagccagttaccttcggaaaaagagttg
gtagctcttgatccggcaaacaaaccaccgctggtagcggtggtttttttgtttgcaagcagcagattacgcgcagaaaaaaaggatctca
agaagatcctttgatcttttctacggggtctgacgctcagtggaacgaaaactcacgttaagggattttggtcatgagattatcaaaaaggat
cttcacctagatccttttaaattaaaaatgaagttttaaatcaatctaaagtatatatgagtaaacttggtctgacagttaccaatgcttaatcag
tgaggcacctatctcagcgatctgtctatttcgttcatccatagttgcctgactccccgtcgtgtagataactacgatacgggagggcttacca
tctggccccagtgctgcaatgataccgcgagacccacgctcaccggctccagatttatcagcaataaaccagccagccggaagggcc
gagcgcagaagtggtcctgcaactttatccgcctccatccagtctattaattgttgccgggaagctagagtaagtagttcgccagttaatagt
ttgcgcaacgttgttgccattgctacaggcatcgtggtgtcacgctcgtcgtttggtatggcttcattcagctccggttcccaacgatcaaggc
gagttacatgatcccccatgttgtgcaaaaaagcggttagctccttcggtcctccgatcgttgtcagaagtaagttggccgcagtgttatcac
tcatggttatggcagcactgcataattctcttactgtcatgccatccgtaagatgcttttctgtgactggtgagtactcaaccaagtcattctgag
aatagtgtatgcggcgaccgagttgctcttgcccggcgtcaatacgggataataccgcgccacatagcagaactttaaaagtgctcatca
ttggaaaacgttcttcggggcgaaaactctcaaggatcttaccgctgttgagatccagttcgatgtaacccactcgtgcacccaactgatctt
cagcatcttttactttcaccagcgtttctgggtgagcaaaaacaggaaggcaaaatgccgcaaaaaagggaataagggcgacacgga
aatgttgaatactcatactcttcctttttcaatattattgaagcatttatcagggttattgtctcatgagcggatacatatttgaatgtatttagaaaa
ataaacaaataggggttccgcgcacatttccccgaaaagtgccacctgacgtctaagaaaccattattatcatgacattaacctataaaa
ataggcgtatcacgaggccctttcgtctcgcgcgtttcggtgatgacggtgaaaacctctgacacatgcagctcccggagacggtcacag
cttgtctgtaagcggatgccgggagcagacaagcccgtcagggcgcgtcagcgggtgttggcgggtgtcggggctggcttaactatgcg
gcatcagagcagattgtactgagagtgcaccattcgacgctctcccttatgcgactcctgcattaggaagcagcccagtagtaggttgag
gccgttgagcaccgccgccgcaaggaatggtgcatgcaaggagatggcgcccaacagtcccccggccacggggcctgccaccata
cccacgccgaaacaagcgctcatgagcccgaagtggcgagcccgatcttccccatcggtgatgtcggcgatataggcgccagcaacc
gcacctgtggcgccggtgatgccggccacgatgcgtccggcgtagaggatctggctagcgatgaccctgctgattggttcgctgaccattt
ccgggtgcgggacggcgttaccagaaactcagaaggttcgtccaaccaaaccgactctgacggcagtttacgagagagatgataggg
tctgcttcagtaagccagatgctacacaattaggcttgtacatattgtcgttagaacgcggctacaattaatacataaccttatgtatcataca
catacgatttaggtgacactatagaatacacggaattaattc
Bold = ITR sequence
Italics = mini-human hP0 promoter
Underline = SH3TC2
Italic underline = synthetic minimal polyA
SEQ ID NO. 22: Mini-human hP0 promoter
tctctcaggcaaggaggctgaggagaaatccctttttatggcctttaaattgaggttccatatctatccctcagagaagtgtgtctgtgtccctg
tttttgtccctctccctcaccaccccccacaacattccagcctggggcagggggaggccagtggacacaaagccctctgtgtatggggtg
gtatgtgtccccccacccctccacccagactatacaatgccccttctgctccctgcactctgcccccctccccaccacctctcaactgcaca
tgccaggctgcaattggttactggctgaggacagccccctcatgctggggccctaggggattttaagcaggttccaggaaccccccgttc
agttcctggtcccccactttctcaaccccacagatgctccgggcccctgcccctgccccagc
SEQ ID NO. 23: AAV-human-miniMpz-Egfp
tagctgcgcgctcgctcgctcactgaggccgcccgggcaaagcccgggcgtcgggcgacctttggtcgcccggcctcagt
gagcgagcgagcgcgcagagagggagtggccaactccatcactaggggttccttgtagttaatgattaacccgccatgcta
cttatctacgtagccatgctctaggtacctctctcaggcaaggaggctgaggagaaatccctttttatggcctttaaattgaggttccatat
ctatccctcagagaagtgtgtctgtgtccctgtttttgtccctctccctcaccaccccccacaacattccagcctggggcagggggaggcca
gtggacacaaagccctctgtgtatggggtggtatgtgtccccccacccctccacccagactatacaatgccccttctgctccctgcactctg
cccccctccccaccacctctcaactgcacatgccaggctgcaattggttactggctgaggacagccccctcatgctggggccctagggg
attttaagcaggttccaggaaccccccgttcagttcctggtcccccactttctcaaccccacagatgctccgggcccctgcccctgccccag
caccggtcgccaccatggtgagcaagggcgaggagctgttcaccggggtggtgcccatcctggtcgagctggacggcgacgtaaacg
gccacaagttcagcgtgtccggcgagggcgagggcgatgccacctacggcaagctgaccctgaagttcatctgcaccaccggcaag
ctgcccgtgccctggcccaccctcgtgaccaccctgacctacggcgtgcagtgcttcagccgctaccccgaccacatgaagcagcacg
acttcttcaagtccgccatgcccgaaggctacgtccaggagcgcaccatcttcttcaggcagacggcaactacaagacccgcgccga
ggtgaagttcgagggcgacaccctggtgaaccgcatcgagctgaagggcatcgacttcaaggaggacggcaacatcctggggcaca
agctggagtacaactacaacagccacaacgtctatatcatggccgacaagcagaagaacggcatcaaggtgaacttcaagatccgc
cacaacatcgaggacggcagcgtgcagctcgccgaccactaccagcagaacacccccatcggcgacggccccgtgctgctgcccg
acaaccactacctgagcacccagtccgccctgagcaaagaccccaacgagaagcgcgatcacatggtcctgctggagttcgtgaccg
ccgccgggatcactctcggcatggacgagctgtacaagtaaagcggccctagatcaagcttatcgataatcaacctctggattacaaaa
tttgtgaaagattgactggtattcttaactatgttgctccttttacgctatgtggatacgctgctttaatgcctttgtatcatgctattgcttcccgtatg
gctttcattttctcctccttgtataaatcctggttgctgtctctttatgaggagttgtggcccgttgtcaggcaacgtggcgtggtgtgcactgtgttt
gctgacgcaacccccactggttggggcattgccaccacctgtcagctcctttccgggactttcgctttccccctccctattgccacggcgga
actcatcgccgcctgccttgcccgctgctggacaggggctcggctgttgggcactgacaattccgtggtgttgtcggggaaatcatcgtcct
ttccttggctgctcgcctgtgttgccacctggattctgcgcgggacgtccttctgctacgtcccttcggccctcaatccagcggaccttccttcc
cgcggcctgctgccggctctgcggcctcttccgcgtcttcgccttcgccctcagacgagtcggatctccctttgggccgcctccccgcatcg
ataccgtcgactcgctgatcagcctcgactgtgccttctagttgccagccatctgttgtttgcccctcccccgtgccttccttgaccctggaagg
tgccactcccactgtcctttcctaataaaatgaggaaattgcatcgcattgtctgagtaggtgtcattctattctggggggtggggtggggca
ggacagcaagggggaggattgggaagacaatagcaggcatgctggggatgcggtgggctctatggcttctgaggcggaaagaacc
agctggggctcgactagagcatggctacgtagataagtagcatggcgggttaatcattaactacaaggaacccctagtgatgga
gttggccactccctctctgcgcgctcgctcgctcactgaggccgggcgaccaaaggtcgcccgacgcccgggctttgcccg
ggcggcctcagtgagcgagcgagcgcgcagagctttttgcaaaagcctaggcctccaaaaaagcctcctcactacttctggaata
gctcagaggccgaggcggcctcggcctctgcataaataaaaaaaattagtcagccatggggcggagaatgggcggaactgggcgg
agttaggggcgggatgggcggagttaggggcgggactatggttgctgactaattgagatgcatgctttgcatacttctgcctgctggggag
cctggggactttccacacctggttgctgactaattgagatgcatgctttgcatacttctgcctgctggggagcctggggactttccacacccta
actgacacacattccacagctgcattaatgaatcggccaacgcgcggggagaggcggtttgcgtattgggcgctcttccgcttcctcgctc
actgactcgctgcgctcggtcgttcggctgcggcgagcggtatcagctcactcaaaggcggtaatacggttatccacagaatcagggga
taacgcaggaaagaacatgtgagcaaaaggccagcaaaaggccaggaaccgtaaaaaggccgcgttgctggcgtttttccataggc
tccgcccccctgacgagcatcacaaaaatcgacgctcaagtcagaggtggcgaaacccgacaggactataaagataccaggcgtttc
cccctggaagctccctcgtgcgctctcctgttccgaccctgccgcttaccggatacctgtccgcctttctcccttcgggaagcgtggcgctttc
tcatagctcacgctgtaggtatctcagttcggtgtaggtcgttcgctccaagctgggctgtgtgcacgaaccccccgttcagcccgaccgct
gcgccttatccggtaactatcgtcttgagtccaacccggtaagacacgacttatcgccactggcagcagccactggtaacaggattagca
gagcgaggtatgtaggcggtgctacagagttcttgaagtggtggcctaactacggctacactagaagaacagtatttggtatctgcgctct
gctgaagccagttaccttcggaaaaagagttggtagctcttgatccggcaaacaaaccaccgctggtagcggtggtttttttgtttgcaagc
agcagattacgcgcagaaaaaaaggatctcaagaagatcctttgatcttttctacggggtctgacgctcagtggaacgaaaactcacgtt
aagggattttggtcatgagattatcaaaaaggatcttcacctagatccttttaaattaaaaatgaagttttaaatcaatctaaagtatatatgag
taaacttggtctgacagttaccaatgcttaatcagtgaggcacctatctcagcgatctgtctatttcgttcatccatagttgcctgactccccgtc
gtgtagataactacgatacgggagggcttaccatctggccccagtgctgcaatgataccgcgagacccacgctcaccggctccagattt
atcagcaataaaccagccagccggaagggccgagcgcagaagtggtcctgcaactttatccgcctccatccagtctattaattgttgccg
ggaagctagagtaagtagttcgccagttaatagtttgcgcaacgttgttgccattgctacaggcatcgtggtgtcacgctcgtcgtttggtatg
gcttcattcagctccggttcccaacgatcaaggcgagttacatgatcccccatgttgtgcaaaaaagcggttagctccttcggtcctccgat
cgttgtcagaagtaagttggccgcagtgttatcactcatggttatggcagcactgcataattctcttactgtcatgccatccgtaagatgcttttc
tgtgactggtgagtactcaaccaagtcattctgagaatagtgtatgcggcgaccgagttgctcttgcccggcgtcaatacgggataatacc
gcgccacatagcagaactttaaaagtgctcatcattggaaaacgttcttcggggcgaaaactctcaaggatcttaccgctgttgagatcca
gttcgatgtaacccactcgtgcacccaactgatcttcagcatcttttactttcaccagcgtttctgggtgagcaaaaacaggaaggcaaaat
gccgcaaaaaagggaataagggcgacacggaaatgttgaatactcatactcttcctttttcaatattattgaagcatttatcagggttattgtc
tcatgagcggatacatatttgaatgtatttagaaaaataaacaaataggggttccgcgcacatttccccgaaaagtgccacctgacgtcta
agaaaccattattatcatgacattaacctataaaaataggcgtatcacgaggccctttcgtctcgcgcgtttcggtgatgacggtgaaaacc
tctgacacatgcagctcccggagacggtcacagcttgtctgtaagcggatgccgggagcagacaagcccgtcagggcgcgtcagcgg
gtgttggcgggtgtcggggctggcttaactatgcggcatcagagcagattgtactgagagtgcaccattcgacgctctcccttatgcgactc
ctgcattaggaagcagcccagtagtaggttgaggccgttgagcaccgccgccgcaaggaatggtgcatgcaaggagatggcgccca
acagtcccccggccacggggcctgccaccatacccacgccgaaacaagcgctcatgagcccgaagtggcgagcccgatcttcccca
tcggtgatgtcggcgatataggcgccagcaaccgcacctgtggcgccggtgatgccggccacgatgcgtccggcgtagaggatctggc
tagcgatgaccctgctgattggttcgctgaccatttccgggtgcgggacggcgttaccagaaactcagaaggttcgtccaaccaaaccga
ctctgacggcagtttacgagagagatgatagggtctgcttcagtaagccagatgctacacaattaggcttgtacatattgtcgttagaacgc
ggctacaattaatacataaccttatgtatcatacacatacgatttaggtgacactatagaatacacggaattaattc
Bold = ITR sequence
Italics = mini-human hP0 promoter
Underline = EGFP
Italic underline = WPRE sequence
SEQ ID NO. 24: Minimal synthetic poly A sequence
ggagctctcgagaggcctaataaagagctcagatgcatcgatcagagtgtgttggttttttgtgtgagatct

### EXAMPLES

The invention shall now be described with reference to the following non-limiting examples.

### Example 1: AAV Transfer Plasmid Cloning

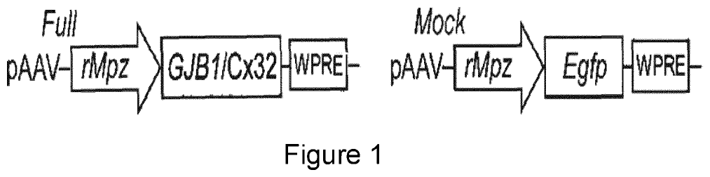

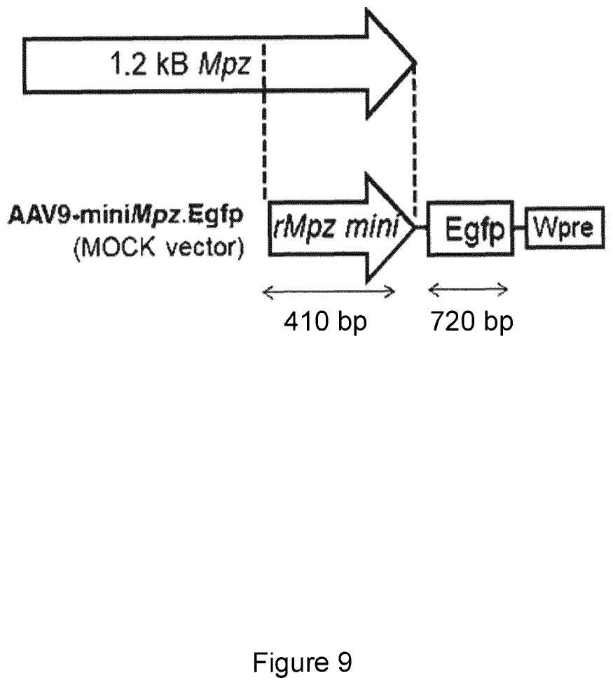

AAV vectors were designed to provide Schwann cell-specific expression of Cx32 (pAAV-Mpz.GJB1, full vector) or of the reporter gene EGFP (pAAV-Mpz.Egfp, mock vector), both under the 1.127 kB Mpz promoter shown to drive expression specifically in Schwann cells (26, 32). These vectors were cloned using as starting plasmid the AAV construct pAM/Mbp-EGFP-WPRE-bGH (57), containing the woodchuck hepatitis virus post-transcriptional regulatory element (WPRE) and the bovine growth hormone polyadenylation sequence (bGHpA) flanked by AAV2 inverted terminal repeats (FIGS. 1 and 9).

Specific details of how the three constructs AAV-Mpz.Egfp, AAV-Mpz.GJB1 and AAV-miniMpz.Egfp were cloned are as follows:

### 264-P0-EGFP-WPRE (=AAV-Mpz.Eafp-SEQ ID NO. 1)

pBluescript SK+ plasmid that contains the Mpz promoter sequence was used in order to digest out the promoter sequence using XhoI and EcoRV restriction enzymes. The AAV vector was also digested using the same enzymes. After ligation and transformation correct assembly of the expression cassette was confirmed by restriction digest mapping and direct sequencing using primers covering the entire coding sequence.

### 264-Mpz(P0)-Cx32-WPRE (=AAV-Mpz.GJB1—SEQ ID NO. 2)

The Mpz/Cx32 ORF was PCR amplified from a lentiviral construct previously made. The primers used for the amplification were P0-Cx32-F 5′-AGGGGTACCCTTCCTGTTCAGACT-3′ (SEQ ID NO. 13) and P0-Cx32-R 5′-CCGCTCGAGGGATCCTC AGCAG-3′ (SEQ ID NO. 14). The PCR product (2030 bp) was gel purified using the Qiagen gel extraction kit and digested with KpnI and XhoI. The AAV vector was also digested with the same restriction enzymes. The entire expression cassette was confirmed by direct sequencing of the ORFs.

### 264-Mpz(P0) Min-EGFP-WPRE (=AAV-miniMpz.Eafp—SEQ ID NO. 3)

The AAV vector 264 was digested with HindIII and was self-ligated. Then a linker was inserted to the vector. Mpzmin was PCR amplified from the rat Mpz promoter sequence, using the following primers: KpnI-P0-F: 5′-GGGGTACCGCTCTCAGGCAAG-3′ (SEQ ID NO. 15) and AgeI-P0-R: 5′-AAACCGGTTGGCAGAGCGTCTGT-3′ (SEQ ID NO. 16). The insert (420 bp) was then directionally cloned to our AAV vector 264. EGFP was digested from another construct using AgeI and HindIII and was directly ligated in.

### Example 2: AAV Vector Production, Purification and Titration

The production of AAV9 vectors was performed according to published protocols (58). The pAAV-Mpz.Egfp and pAAV-Mpz.GJB1 plasmids were cross-packaged into AAV9 capsid (capsid plasmids provided by Dr. A. Bosch, University of Barcelona, Spain, and originally developed by Dr. James Wilson, University of Pennsylvania Vector Core, PA, USA).

AAV viral stocks for pseudotypes 9 were generated as previously described (59). Recombinant AAV (rAAV) vectors were produced by triple transfection of 2×108 HEK293 cells with 250 μg of pAAV, 250 μg of pRepCap, and 500 μg of pXX6 plasmid mixed with polyethylenimine (PEI; branched, MW 25,000; Sigma). Briefly, 48 hr after transfection, cells were harvested by centrifugation (200 g, 10 min); resuspended in 30 ml of 20 mM NaCl, 2 mM MgCl2, and 50 mM Tris-HCl (pH 8.5) and lysed by three freeze-thaw cycles. Cell lysate were clarified by centrifugation (2000 g, 10 min) and rAAV particles were purified from the supernatant by iodixanol gradient as follows: The clarified lysate was treated with 50 U/ml of Benzonase (Novagen; 1 hr, 37 °C) and centrifuged (3000 g, 20 min). The vector-containing supernatant was collected and adjusted to 200 mM NaCl using a 5 M stock solution. To precipitate the virus from the clarified cell lysate, polyethylene glycol (PEG 8000; Sigma) were added to a final concentration of 8% and the mixture incubated (3 hr, 4 °C) and centrifuged (8000 g, 15 min). The rAAV-containing pellets were resuspended in 20 mM NaCl, 2 mM MgCl2, and 50 mM Tris-HCl (pH 8.5) and incubated for 48 hr at 4 °C. rAAV particles will be purified using the iodixanol method as described (59). If necessary, rAAV was concentrated and desalted in PBSMK using Amicon Ultra-15 Centrifugal Filter Device (Millipore). Titration was evaluated by picogreen quantification (60) and calculated as viral genomes per milliliter (vg/ml).

### Example 3: Intrathecal Vector Delivery

Following a small skin incision along the lower lumbar spine level to visualize the spine, the AAV vector was delivered into the L5-L6 intervertebral space of anesthetized mice at a slow rate of 5 μl/min. A 50-μL Hamilton syringe (Hamilton, Giarmata, Romania) connected to a 26-gauge needle was used to inject a total volume of 20 μL containing 0.5-1×10¹¹ vector genomes (vg) of the AAV vector. A flick of the tail was considered indicative of successful intrathecal administration.

### Example 4: AAV9-Mediated Schwann-Cell Targeted Gene Expression

2 month old wild-type mice were treated with the AAV9-Mpz-Egfp vector described in Examples 1 and 3 above. Samples were analyzed by DNA extraction from PNS tissues and determination of the presence of the viral DNA measured as vector copy numbers (VCNs) 4 and 6 weeks post-injection (Table 1) as we previously described (33). Immunofluorescence staining of lumbar root sections and immunoblot of lumbar root, femoral nerves and sciatic nerves were also carried out as described below 4 and 8 weeks post-injection (Table 2).

Immunofluorescence staining: For immunostaining, mice were anesthetized with avertin according to institutionally approved protocols, and then transcardially perfused with normal saline followed by fresh 4% paraformaldehyde in 0.1 M PB buffer. The lumbar-sacral spinal cords with spinal roots attached, as well as the bilateral sciatic and femoral motor nerves were dissected. All tissues were frozen for cryosections, while sciatic and femoral nerves were isolated and teased into fibers under a stereoscope. Teased fibers or sections were permeabilized in cold acetone and incubated at RT with a blocking solution of 5% BSA (Sigma-Aldrich, Munich, Germany) containing 0.5% Triton-X (Sigma-Aldrich, Munich, Germany) for 1 h. Primary antibodies used were: mouse monoclonal antibody against contactin-associated protein (Caspr, 1:50; gift of Dr Elior Peles, Weizmann Institute of Science), rabbit antisera against EGFP (1:1,000; Invitrogen, USA), Capr2 (1:200, Alomone Labs, Israel) and Cx32 (1:50; Sigma, Munich, Germany) all diluted in blocking solution and incubated overnight at 4 °C. Slides were then washed in PBS and incubated with fluorescein- and rhodamine-conjugated mouse and rabbit cross-affinity purified secondary antibodies (1:500; Jackson ImmunoResearch, USA) for 1 h at RT. Cell nuclei were visualized with DAPI (1 μg/ml; Sigma, Munich, Germany). Slides were mounted with fluorescent mounting medium and images photographed under a fluorescence microscope with a digital camera using Axiovision software (Carl Zeiss MicroImaging; Oberkochen, Germany).

Expression rates for the Egfp reporter gene were quantified by counting the number of EGFP-positive Schwann cells as a percentage of total Schwann cells in lumbar roots and sciatic nerves. Expression of Cx32 was quantified by visualizing nodal areas of myelinated fibers with axonal domain markers including juxtaparanodal Kv1.2 and paranodal Caspr in double staining with Cx32. The number of nodal areas positive for Cx32 immunoreactivity was counted as a percentage of total nodal areas in lumbar roots and sciatic nerves.

Immunoblot analysis: Immunoblot analysis of root and peripheral nerve lysates was used to detect the expression of either the reporter gene Egfp or Cx32 in tissues of injected mice. Immunoblots of lumbar root, femoral and sciatic nerve lysates collected 4 weeks post-injection were incubated with rabbit anti-Egfp (1:1000; Abcam) and anti-Cx32 (clone 918, 1:3,000) primary antibodies followed by HRP-conjugated anti-rabbit secondary antibodies (Jackson ImmunoResearch, diluted 1:3,000). The bound antibody was visualized by an enhanced chemiluminescence system (GE Healthcare Life Sciences).

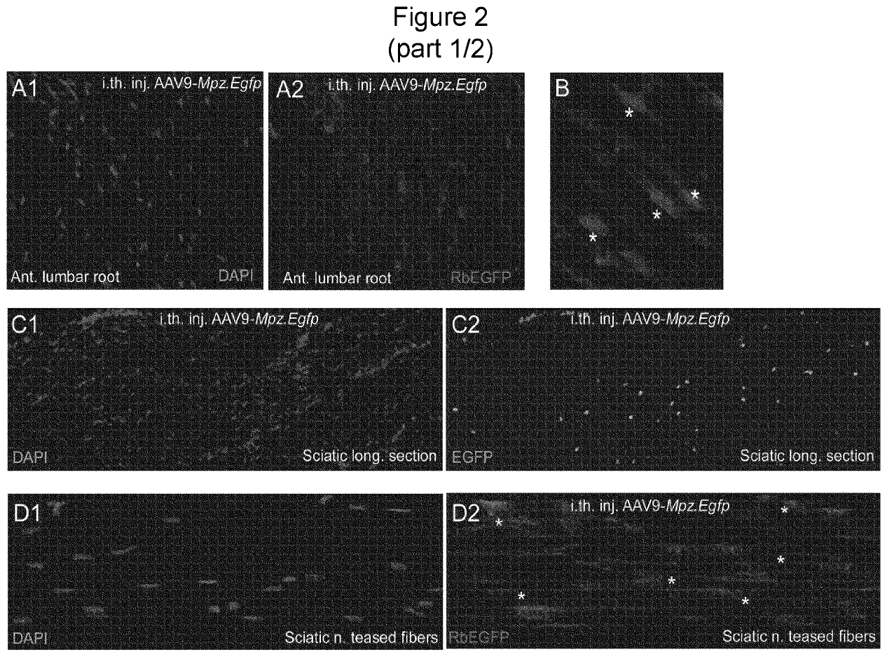

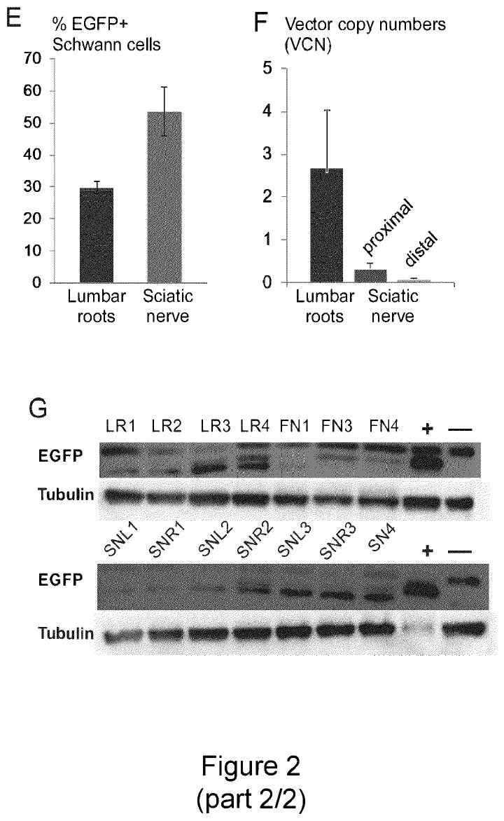

Results are shown in FIG. 2 and Tables 1 and 2 below. It was possible to detect high expression levels of the reporter gene EGFP (enhanced green fluorescent protein) specifically in Schwann cells, the myelinating cells of the PNS, including the lumbar spinal nerve roots and distal sciatic nerves and this shows specific expression of the EGFP reporter gene in lumbar root and sciatic nerve samples, indicating that tissue specific expression in Schwann cells is achieved using this vector delivery system.

TABLE 1
Vector copy numbers (VCN) in all tissues examined in WT mice injected with AAV9-Mpz.Egfp:

| | 4 weeks | | | 6 weeks | | | 8 weeks | | |
|---|---|---|---|---|---|---|---|---|---|
| | Lumbar | Sciatic nerve | Sciatic nerve | Lumbar | Sciatic nerve | Sciatic nerve | Lumbar | Sciatic nerve | Sciatic nerve |
| | roots | proximal | distal | roots | proximal | distal | roots | proximal | distal |
| VCN | 2.66 ± 1.37 | 0.31 ± 0.13 | 0.07 ± 0.03 | 0.62 ± 0.28 | 0.23 ± 0.13 | 0.02 ± 0.01 | 0.24 ± 0.1 | 0.82 ± 0.45 | 0.18 ± 0.08 |
| N | 4 | 2 | 4 | 4 | 4 | 4 | 3 | 4 | 4 |
| number of | | | | | | | | | |
| mice | | | | | | | | | |

TABLE 2
EGFP expression rates (% Egfp-postive Schwann cells)
in lumbar roots and sciatic nerves of AAV9-Mpz.EGFP
injected WT mice 4- and 8-weeks post-injection:

| | Sciatic | | Roots | |
|---|---|---|---|---|
| | 4 weeks | 8 weeks | 4 weeks | 8 weeks |
| EGFP expression | 53.6 ± 7.66 | 39.9 ± 2.49 | 29.7 ± 1.89 | 35.0 ± 3.23 |
| N | 4 | 3 | 4 | 3 |

### Example 5: Expression of Intrathecally Delivered AAV9-Mpz.GJB1 Vector in 2-Month Cx32KO and R75W KO Mice

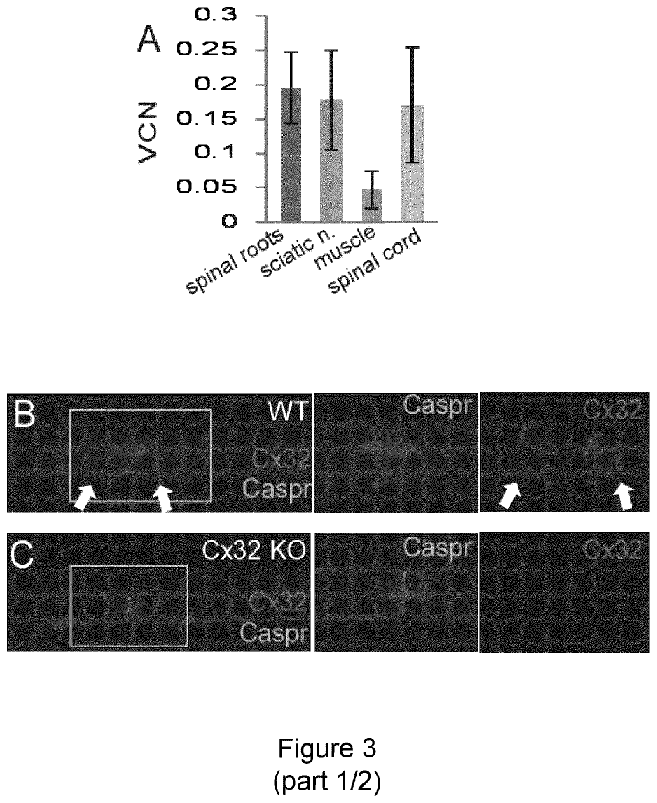

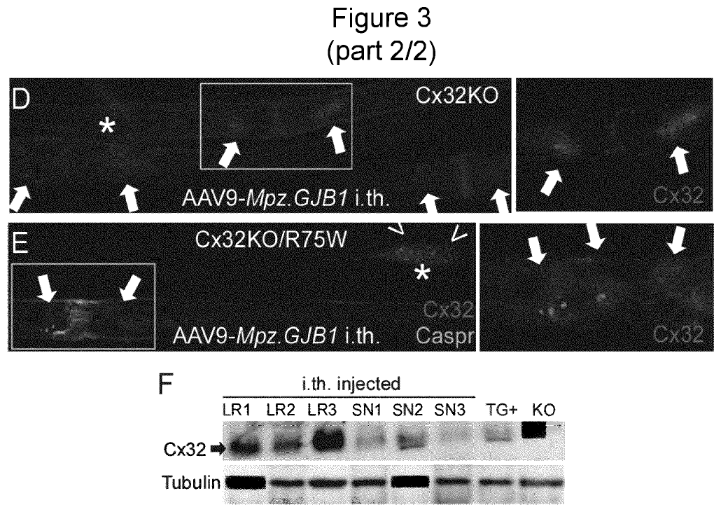

The AAV9-Mpz.GJB1 vector was produced as described in Example 1 above (5×10¹² vg/ml) and delivered to 2- and 6-month old Cx32 KO mice by lumbar intrathecal (i.th.) injection (5×10¹⁰ vg in 20 μl). Analysis of VCNs from DNA extracted from PNS tissues as previously described (33) per cell in different tissues revealed widespread biodistribution (FIG. 3A), including in spinal roots and sciatic nerves with highest levels in the liver (55). Immunostaining and immunoblot analysis were carried out as described above. Cx32 was expressed at paranodal myelin areas in over 60-70% of myelinating Schwann cells in lumbar spinal roots and in sciatic nerve fibers (FIGS. 3B-D). AAV9-delivered Cx32 expression at high levels could be also detected by Western blot of PNS tissue lysates from injected as opposed to non-injected Cx32 KO mice (FIG. 3F).

In order to clarify whether the AAV9-Mpz.GJB1 viral vector allowing higher expression levels could overcome the interfering effects of Golgi-retained mutants observed with the lentiviral vector in our previous studies (29, 34), the inventors also injected 2-month old R75W knockout (R75W KO) mice. Importantly, paranodal localization of AAV9-delivered Cx32 was also detected in R75W/KO tissues, despite the co-expression of the interfering Golgi-retained R75W mutant showing the typical perinuclear localization (FIG. 3E). Thus, AAV9 shows the potential to provide widespread, high level and Schwann-cell targeted gene expression that may also overcome the interfering effect of a representative Golgi-retained CMT1X mutant.

These results are shown in FIG. 3 and Tables 3 and 4 below, demonstrating that using the vectors to deliver copies of the wild-type GJB1 gene results in successful expression of Cx32 in both Cx32 knockout mice and R75W knockout mice. The R75W Golgi-retained mutant (FIG. 3E) also achieves expression of Cx32 despite the presence of R75W Cx32 mutant protein in perinuclear areas, whereas this had not been possible in previous work not using the AAV vector.

TABLE 3
Vector copy numbers in all tissues examined in Cx32 KO
mice injected with AAV9-Mpz.GJB1:

| | Lumbar roots | Sciatic nerve | Muscle | Spinal cord |
|---|---|---|---|---|
| VCN | 0.20 ± 0.05 | 0.18 ± 0.07 | 0.05 ± 0.03 | 0.17 ± 0.08 |
| N | 12 | 12 | 3 | 5 |

TABLE 4
Cx32 expression rates (% Cx32-positive paranodal myelin areas) in lumbar roots
and sciatic nerves of AAV9-Mpz.GJB1 injected 2-and 6-month-old Cx32 KO and Cx32
KO/R75W transgenic mice:

| | Sciatic | | | Lumbar roots | | |
|---|---|---|---|---|---|---|
| | KO 2 mo | KO 6 mo | KO R75W | KO 2 mo | KO 6 mo | KO R75W |
| Cx32 | 71.5 ± 5.8 | 71.7 ± 6.0 | 73.2 ± 5.1 | 63.1 ± 3.1 | 62.7 ± 8.1 | 64.9 ± 3.8 |
| expression | | | | | | |
| N | 6 | 3 | 6 | 6 | 3 | 6 |

### Example 6: Behavioral Analysis of AAV9-Mpz.GJB1 (Full) Injected 6-Mo Old Cx32 KO Mice Compared to AAV9-Mpz.Egfp (Mock) Treated Littermates

Treatment of mice: A gene therapy trial was conducted using two groups of 6-month old Cx32 knockout (KO) mice. A minimum of 8-12 mice per treatment group for each outcome measured was considered adequate for assessing statistically significant differences based on the previous studies using similar models (32, 33). Animals were treated at the age of 6 months, after the onset of the pathology (known to start after 3 months of age).

Littermate mice were randomized to receive either AAV9-Mpz.GJB1 (full) treatment or AAV9-Mpz.Egfp (mock treatment, as a control group) and were assigned a coding number for further identification.

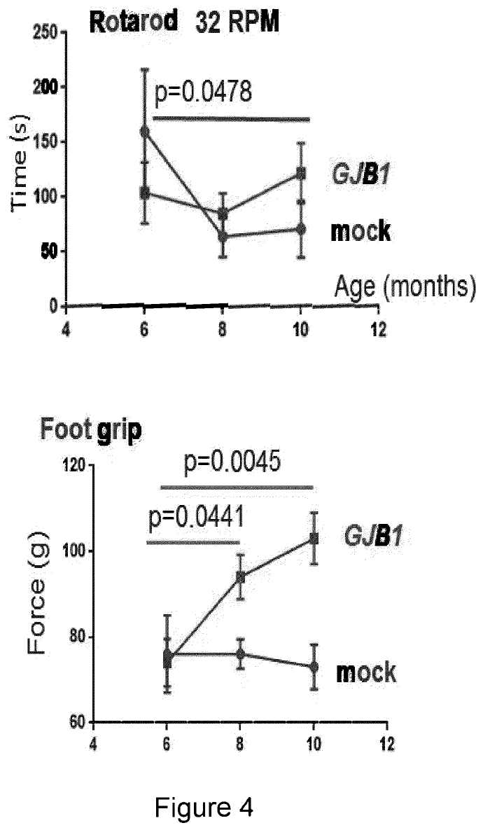

Behavioral testing: Mice were then evaluated by behavioral testing as set out below before treatment, and again at the ages of 8 and 10 months, by an examiner blinded to the treatment condition (FIG. 4 and Table 5).

Rotarod testing: Motor balance and coordination was determined as described previously (61) using an accelerating rotarod apparatus (Ugo Basile, Varese, Italy). Training of animals consisted of three trials per day with 15-min rest period between trials, for 3 consecutive days. The mice were placed on the rod and the speed was gradually increased from 4 to 40 rotations per minute (rpm). Testing was performed on the fourth day using two different speeds, 20 and 32 rpm. Latency to fall was calculated for each speed. The test lasted until the mouse fell from the rod or after the mouse remained on the rod for 600 s and was then removed. Each mouse was placed on the rotarod three times at each speed used and three different values were obtained for each speed. Mean values were used for each mouse at the two different speeds.

Grip strength testing: To measure grip strength, mice were held by the tail and lowered towards the apparatus (Ugo Basile, Varese, Italy) until they grabbed the grid with the hind paws. Mice were gently pulled back until they released the grid. Measurements of the force in g were indicated on the equipment. Each session consisted of three consecutive trials and measurements were averaged. Hind limb force was compared between AAV9.Mpz-GJB1 and AAV9.Mpz-Egfp treated mice.

Older Cx32 KO mice treated with the AAV9-Mpz.GJB1 full therapeutic vector performed significantly better in those tests compared to AAV9-Mpz.Egfp mock (non-therapeutic) vector injected littermates (n=20 mice per group).

Results are shown in FIG. 4 and Table 5 below, which show that motor performance (as measured by both rotarod and foot grip testing) in the GJB1 treated group was improved 2 months after injection (at 8 months of age) and that this improvement remained stable up to 10 months of age. Mock treated mice did not show an improvement in motor performance.

TABLE 5
Longitudinal comparison of motor behavioural performance of Cx32 KO treatment groups:

| | Full treatment group | | | | | |
|---|---|---|---|---|---|---|
| Time point | AAV9.Mpz-GJB1 | | | Mock treatment group | | |
| comparison | (Mann-Whitney test compared to mock) | | | AAV9.Mpz-Egfp | | |
| (months of | Rotarod | Rotarod | Food grip test | Rotarod | Rotarod | Food grip test |
| age) | 20 RPM (sec) | 32 RPM (sec) | (gram) | 20 RPM (sec) | 32 RPM (sec) | (gram) |
| 6 | 317.7 ± 45.62 | 103.5 ± 27.90 | 74.5 ± 5.46 | 345.1 ± 85.60 | 159.7 ± 56.34 | 76.0 ± 9.14 |
| | p > 0.05 | p > 0.05 | p > 0.05 | | | |
| 8 | 241.3 ± 35.92 | 84.6 ± 26.89 | 93.5 ± 5.18 | 207.7 ± 38.43 | 64.5 ± 18.60 | 76.2 ± 3.41 |
| | p > 0.05 | p > 0.05 | p = 0.0196 | | | |
| 10 | 312.7 ± 39.79 | 121.9 ± 27.42 | 103.4 ± 6.01 | 288.9 ± 42.44 | 71.2 ± 25.86 | 73.2 ± 5.15 |
| | p > 0.05 | p = 0.0427 | p = 0.0025 | | | |

### Example 7: Sciatic Nerve Motor Conduction Studies

Cx32 KO mice were treated as described in Example 6 above at the age of 6 months, after the onset of neuropathy, and then motor nerve conduction studies carried out as described below at the age of 10 months.

Motor nerve conduction velocity (MNCV): MNCV was measured in vivo using published methods (62) from bilateral sciatic nerves following stimulation in anesthetized animals at the sciatic notch and distally at the ankle via bipolar electrodes with supramaximal square-wave pulses (5 V) of 0.05 ms. The latencies of the compound muscle action potentials (CMAP) were recorded by a bipolar electrode inserted between digits 2 and 3 of the hind paw and measured from the stimulus artifact to the onset of the negative M-wave deflection. MNCV was calculated by dividing the distance between the stimulating and recording electrodes by the result of subtracting distal from proximal latency.

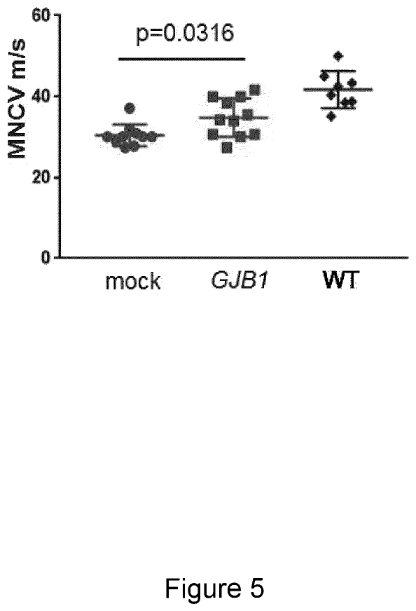

Results from the MNCV study carried out at 10 months of age are shown in FIG. 5 and Table 6 below which shows that motor nerve conduction velocity was improved when measured at 10 months in Cx32 KO mice treated with GJB1, and approaches wild-type levels, compared to the mock treated control group (n=10 mice).

TABLE 6
Motor nerve conduction velocities and amplitude measurements
of AAV9.Mpz.EGFP (Mock, Cx32 KO control group),
AAV9.Mpz.GJB1 (Full, full treatment group) and WT:

| | | | p value | | p value |
|---|---|---|---|---|---|
| | | | mock | | WT |
| | Mock | Full | vs full | WT | vs full |
| MNCV | 30.4 ± 0.87 | 34.8 ± 1.44 | 0.0316 | 41.7 ± 1.62 | 0.0068 |
| (m/s) | | | | | |
| Ampli- | 3.2 ± 0.35 | 4.15 ± 3.22 | >0.05 | 3.3 ± 0.29 | >0.05 |
| tude | | | | | |
| N | 10 | 11 | | 8 | |

### Example 8: Morphological Analysis of Anterior Spinal Roots, Sciatic Nerves and Femoral Nerves of Cx32 KO Mice Following Intrathecal Delivery of the AAV9-Mpz.GJB1 Compared to Mock-Treated Mice Vector

Cx32 KO mice were treated as described in Example 6 above at the age of 6 months and examined 4 months later, at the age of 10 months.

Mice were transcardially perfused with 2.5% glutaraldehyde in 0.1 M PB buffer. The lumbar spinal cord with multiple spinal roots attached, as well as the femoral and sciatic nerves, were dissected and fixed overnight at 4 °C, then osmicated, dehydrated, and embedded in araldite resin (all purchased from Agar Scientific, Essex, UK). Transverse semi-thin sections (1 μm) of the lumbar spinal cord with roots and the middle portion of the femoral motor and sciatic nerves were obtained and stained with alkaline toluidine blue (Sigma-Aldrich, Munich, Germany). Sections were visualized with 10×, 20×, and 40× objective lenses and captured with a Nikon DS-L3 camera (Nikon Eclipse-Ni; Tokyo, Japan). Images of whole root or transverse nerve sections were obtained at 100-200× final magnification, and a series of partially overlapping fields covering the cross-sectional area of the roots or the nerves were captured at 400× final magnification. These images were used to examine the degree of abnormal myelination in both groups as described previously (22, 32, 63). In brief, all demyelinated, remyelinated, and normally myelinated axons were counted using the following criteria: axons larger than 1 μm without a myelin sheath were considered demyelinated, axons with myelin sheaths<10% of the axonal diameter and/or axons surrounded by “onion bulbs” (i.e., circumferentially arranged Schwann cell processes and extracellular matrix) were considered remyelinated, and other myelinated axons were considered normally myelinated.

In addition, the number of foamy macrophages present within the entire cross section of each root or nerve were counted, as an indication of inflammation. Macrophages were identified in semi-thin sections at 400× magnification as cells laden with myelin debris, devoid of a basement membrane, and extending small, microvilli-like processes, as described previously (64, 65). The macrophage count was calculated as the ratio per 1,000 myelinated fibers, to account for size differences between different spinal roots and nerves. All pathological analyses were performed blinded to the treatment condition of each mouse.

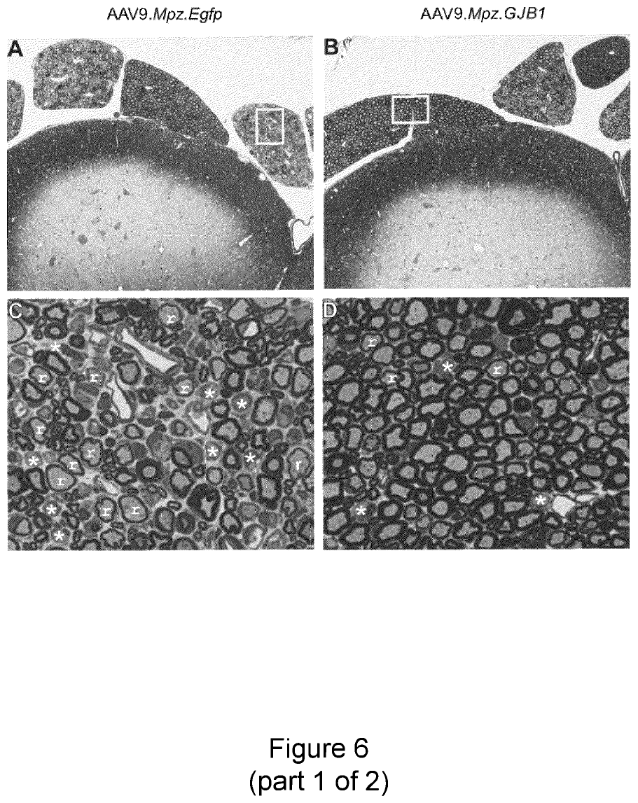

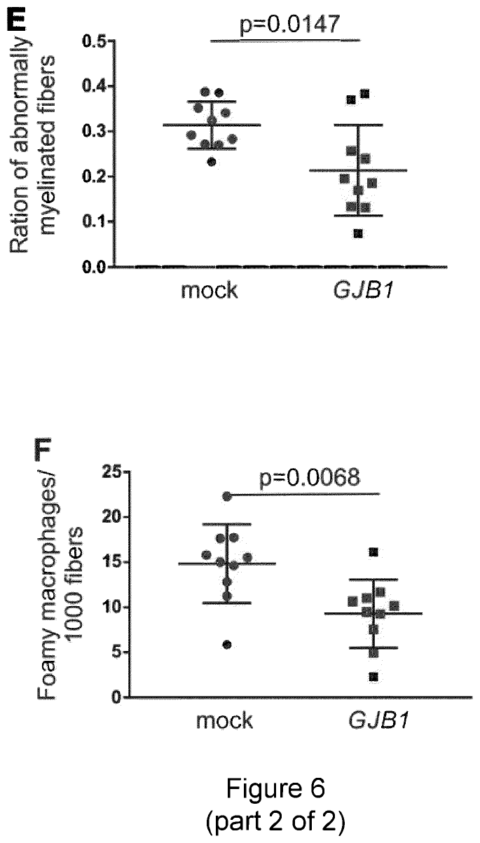

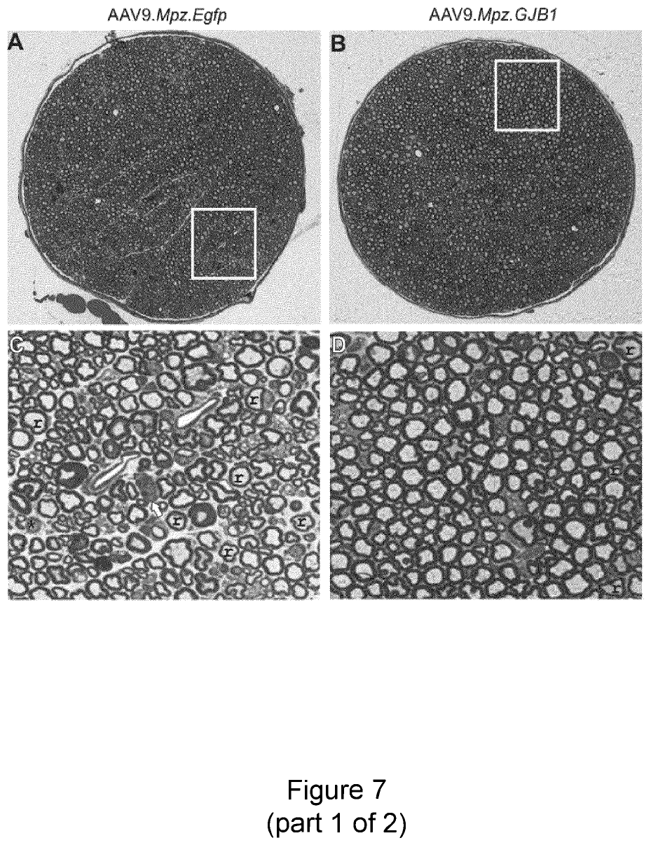

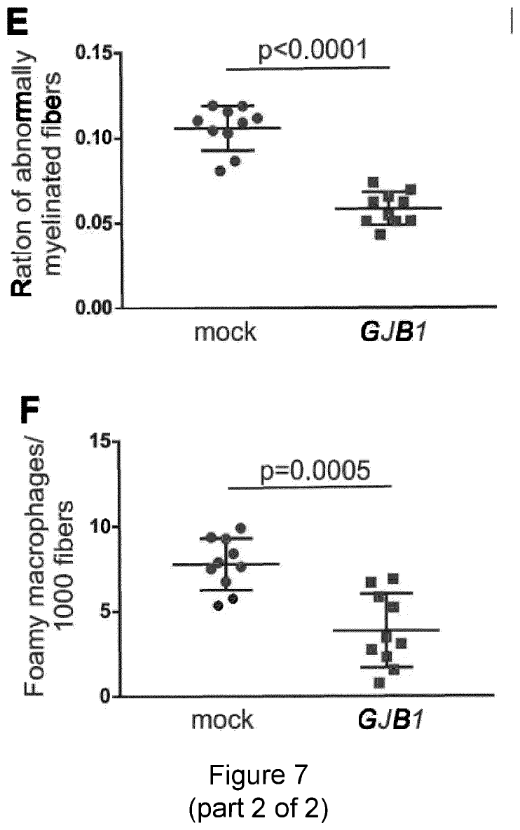

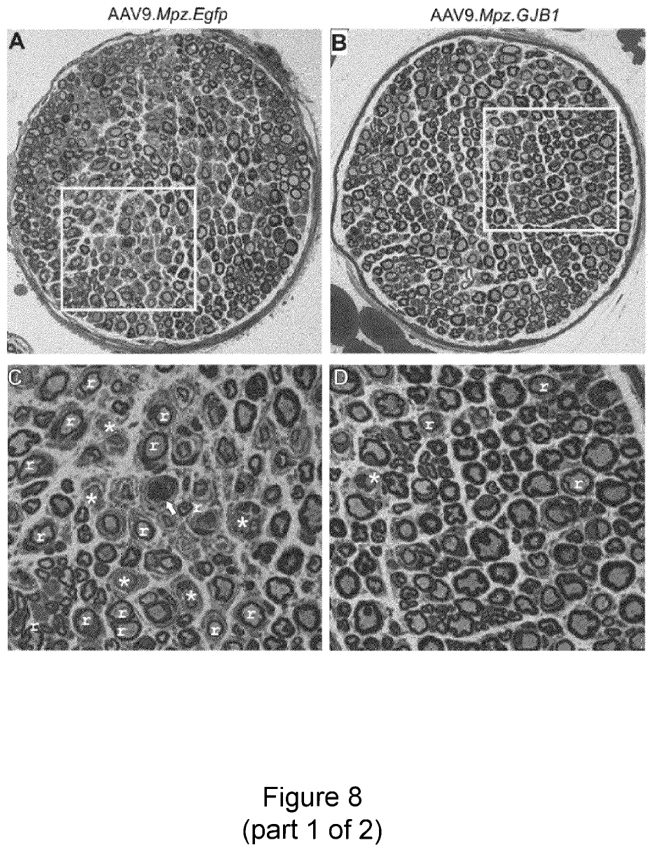

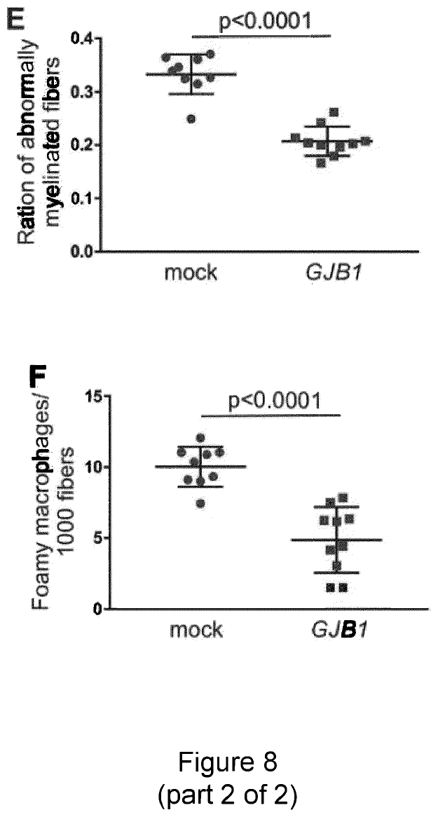

Results are shown in FIG. 6 and Table 7 (for anterior spinal roots), FIG. 7 and Table 8 (for sciatic nerves) and FIG. 8 and Table 9 (for femoral motor nerves). These results show improved myelination of spinal roots, sciatic nerves and femoral nerves compared to the mock-treated control group with fewer demyelinated and re-myelinated fibers, along with an improved ratio of abnormally myelinated fibers. All samples showed a reduction in the number of foamy macrophages in the GJB1 treated group, indicating a reduction in inflammation in the treated group.

TABLE 7
Results of morphometric analysis of anterior lumbar roots in
intrathecally treated Cx32 KO mice at 10 months of age:

| | | | Mann- |
|---|---|---|---|
| | AAV9-Mpz.Egfp | AAV9-Mpz.GJB1 | Whitney |
| | (mock) injected | (full) injected | test |
| Anterior lumbar | (n = 10 mice) | (n = 10 mice) | |
| roots | | | |
| Ratio abnormally | 0.315 ± 0.016 | 0.215 ± 0.032 | p = 0.0147 |
| myelinated fibers | | | |
| Number of | 14.85 ± 1.38 | 9.31 ± 1.20 | p = 0.0068 |
| macrophages/ | | | |
| 1000 fibers | | | |

TABLE 8
Results of morphometric analysis of sciatic nerves in
intrathecally treated Cx32 KO mice at 10 months of age:

| | | | Mann- |
|---|---|---|---|
| | AAV9-Mpz.Egfp | AAV9-Mpz.GJB1 | Whitney |
| | (mock) injected | (full) injected | test |
| Sciatic nerves | (n = 10 mice) | (n = 10 mice) | |
| Ratio abnormally | 0.105 ± 0.004 | 0.058 ± 0.003 | p < 0.0001 |
| myelinated fibers | | | |
| Number of | 7.76 ± 0.48 | 3.84 ± 0.69 | p = 0.0005 |
| macrophages/ | | | |
| 1000 fibers | | | |

TABLE 9
Results of morphometric analysis of femoral motor nerves in
intrathecally treated Cx32 KO mice at 10 months of age:

| | | | Mann- |
|---|---|---|---|
| | AAV9-Mpz.Egfp | AAV9-Mpz.GJB1 | Whitney |
| | injected | injected | test |
| Femoral nerves | (n = 9 mice) | (n = 10 mice) | |
| Ratio abnormally | 0.333 ± 0.012 | 0.207 ± 0.009 | p < 0.0001 |
| myelinated fibers | | | |
| Number of | 10.03 ± 0.47 | 4.87 ± 0.73 | p < 0.0001 |
| macrophages/ | | | |
| 1000 fibers | | | |

### Example 9: Development of AAV Vectors for Schwann Cell Targeted Expression Driven by Minimal Promoter (miniMpz) Elements

The AAV9-based approach described in the above examples has a high potential for clinical translation to treat other demyelinating CMT types including CMT4C. However, the limitation of smaller transgene capacity in AAV vectors needs to be overcome.

In order to facilitate an AAV-mediated Schwann cell targeted gene expression, the inventors cloned a minimal version of the Mpz promoter. Starting from the 1.127 kb full length Mpz promoter (SEQ ID NO. 4) and based on enhancer/ChIP-seq data indicating that functional regulatory elements (Egr2 and Sox10 binding sites) of the full-length Mpz promoter are located within 400 bp upstream of the start codon (56), the inventors selected this strategy to achieve targeted expression in Schwann cells with a minimal size promoter in order remain within the carrying capacity of the AAV vector. The inventors PCR-amplified the 410 bp from the Mpz promoter upstream of the start codon, and then further cloned this miniMpz promoter into the AAV transfer plasmid along with downstream Egfp as a reporter gene and produced the AAV9-miniMpz.Egfp vector (SEQ ID NO. 3 and FIG. 9).

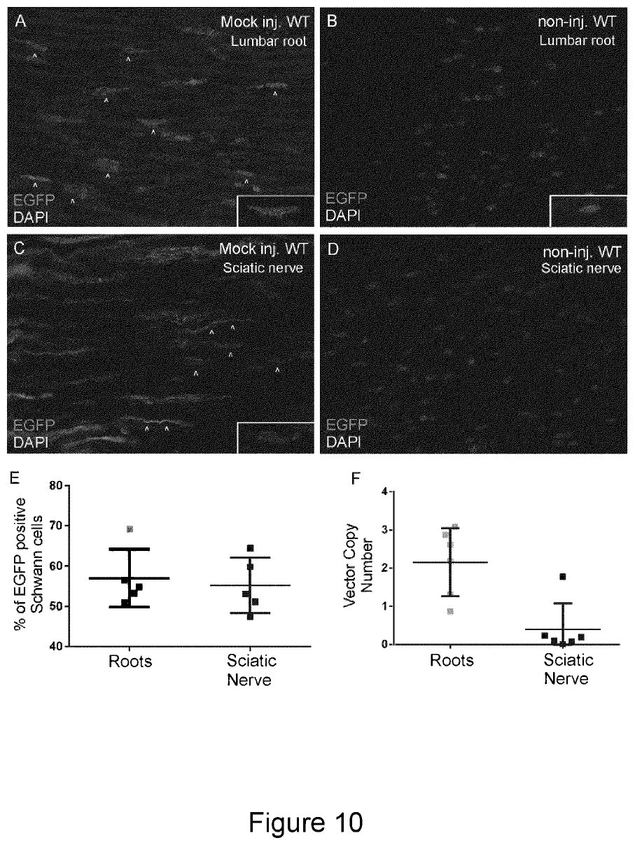

This AAV9-miniMpz.Egfp vector was also validated in vivo in 2-month old wild type (WT) mice using the same delivery method as described in Example 3 by a single lumbar intrathecal injection, and shown to drive expression of reporter gene EGFP in a high percentage of myelinating Schwann cells throughout the PNS. This showed widespread expression of the vector which was mostly restricted to myelinating Schwann cells in PNS tissues, with over 50% expression ratios and high vector copy numbers (VCNs) in lumbar spinal roots and peripheral nerves (FIG. 10).

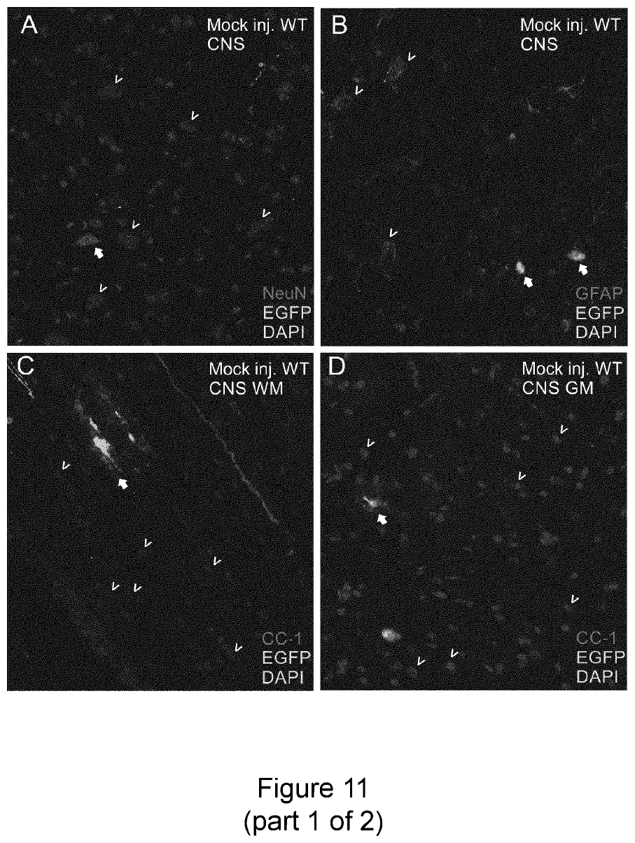

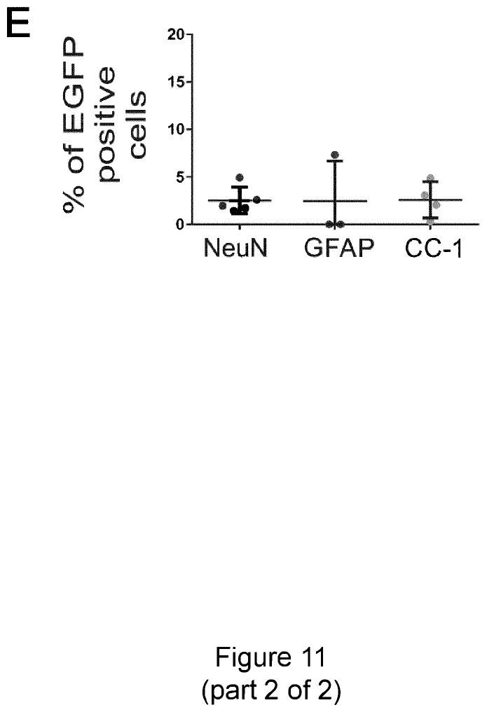

Immunostaining of spinal cord tissue from AAV9-miniMpz-Egfp injected mice that was carried out similarly to as described in Example 4 with cell markers including neuronal NeuN, astrocytic GFAP, and oligodendrocytic CC-1 in white and gray matter combined with EGFP showed expression of the miniMpz-driven construct only in a very small subset of around 2-3% of both neurons and glia cells in the CNS as quantified from n=3-5 mice (FIG. 11).

Results are shown in FIG. 10 (lumbar root and sciatic nerve) and FIG. 11 (lumbar spinal cord), and demonstrate that EGFP expression is distributed adequately in the lumbar root and sciatic nerve and that there is minimal expression in the lumbar spinal cord, showing that after injection there is biodistribution of vector and expression of EGFP reporter protein in Schwann cells in the peripheral nervous system.

### Example 10: Efficacy of Gene Therapy Treatment in a Model of CMT1X when Treated Pre-Onset at Early Stages of the Neuropathy

Groups of 2-month old Cx32 knockout (KO) mice, a model of CMT1X (n=10 mice per group), were injected at the age of 2 months with either the therapeutic (full) AAV9-Mpz-GJB1 vector or with the negative control (mock) vector AAV9-Mpz-Egfp. Behavioral analysis was performed before treatment, and at 4 and 6 months of age. Electrophysiological analysis was carried out at 6 months of age, followed by morphological analysis of semithin sections of peripheral nerve tissues. The same protocols were used as described in Examples 6-8 above, aside from mice were treated at the age of 2 months.

This data provides a model for pre-onset treatment of mice at the early stages of neuropathy (2 months old) in addition to treatment after onset at a later stage of 6 months (Examples 6-8).

### Behavioral Result in Treated Versus Mock-Treated 6-Month Old Cx32 KO Mice

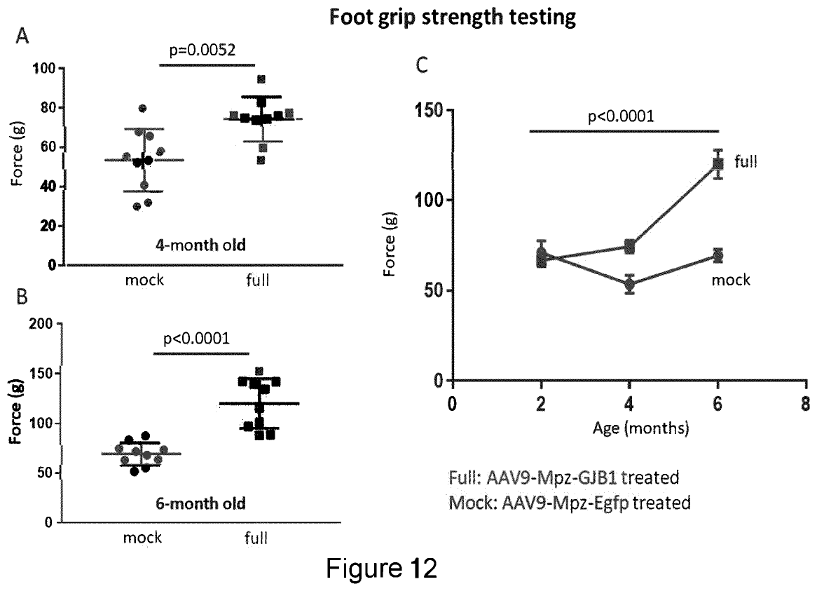

Treatment of groups of 2-month old Cx32 knockout (KO) mice, a model of CMT1X, with either the therapeutic (full) or negative control (mock) vector was performed and mice were examined for motor strength at 4 and 6 months of age. The fully treated group showed significantly improved muscle strength at both time points compared to the mock-treated (FIGS. 12 A and B). The fully treated group also showed significant improvement over time following treatment (FIG. 12C), whereas the mock treated mice did not show any improvement.

### Electrophysiological Studies in Pre-Onset Treated Versus Mock-Treated 6-Month Old Cx32 KO Mice

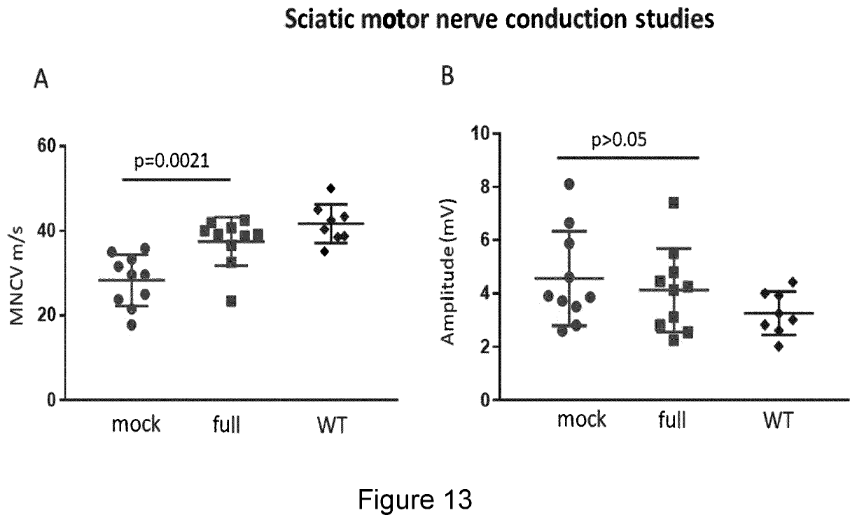

Electrophysiological studies in treated (full) and mock-treated 6-month old Cx32 KO mice showed significant improvement of sciatic nerve conduction velocities after gene therapy treatment are shown in FIG. 13.

FIG. 13 shows a significantly improved sciatic nerve conduction velocities in AAV9-Mpz-GJB1 (full vector) pre-onset treated compared to mock vector treated Cx32 KO mice.

Morphological studies in peripheral nerve tissues in treated versus mock-treated 6-month old Cx32 KO mice

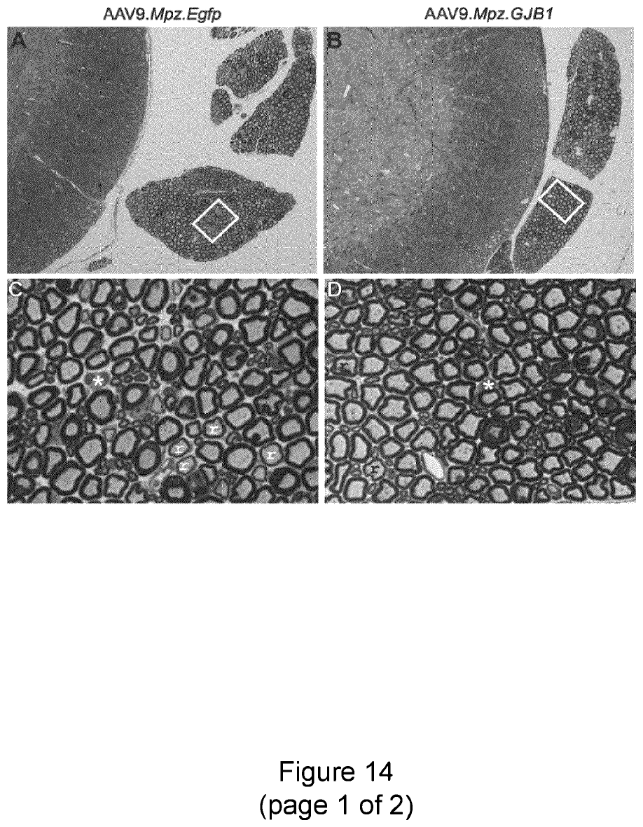

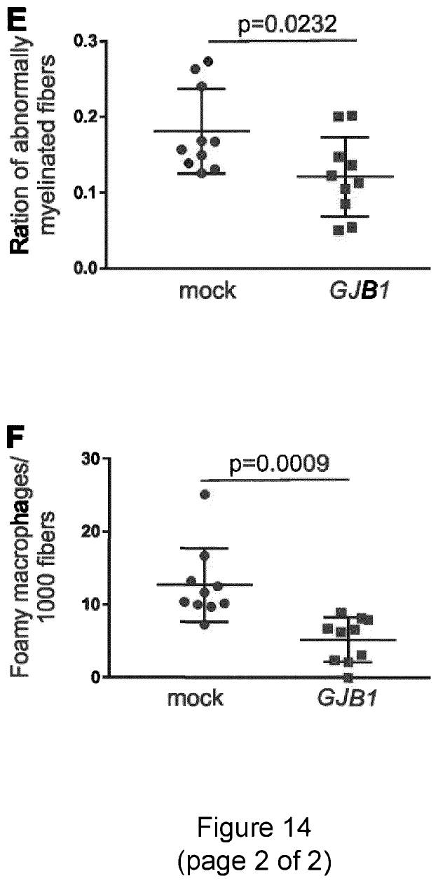

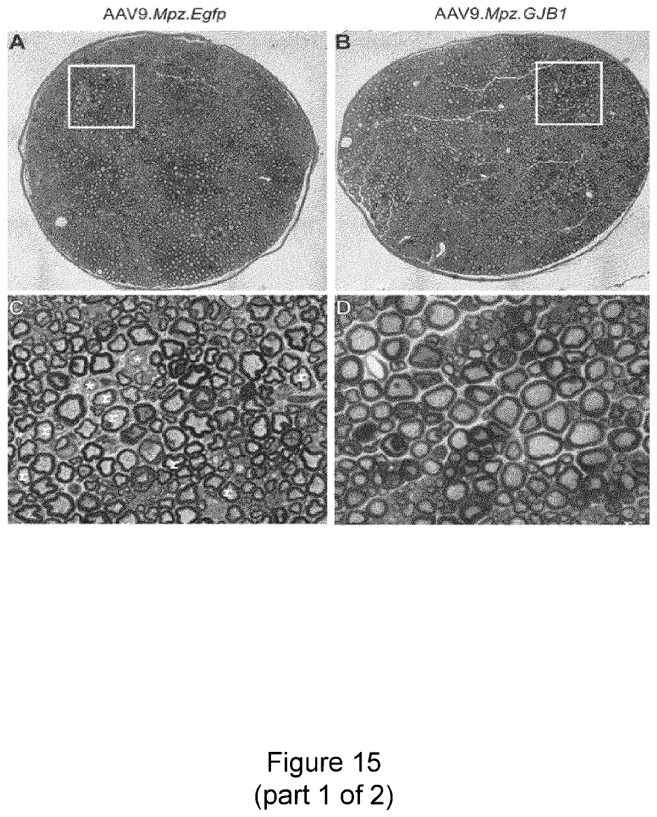

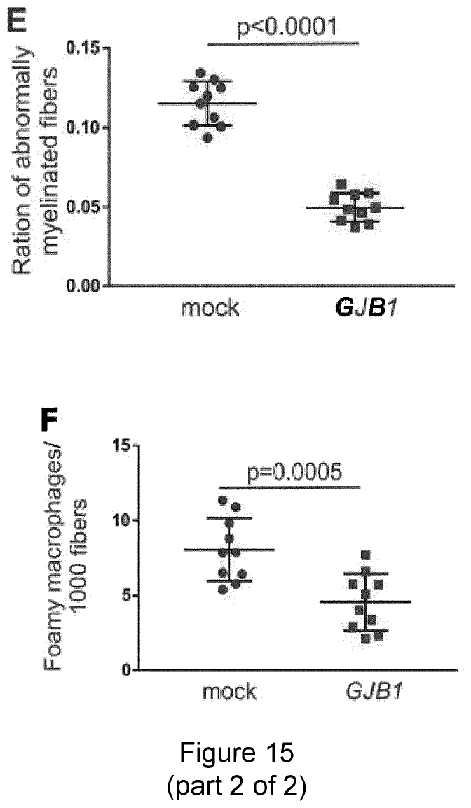

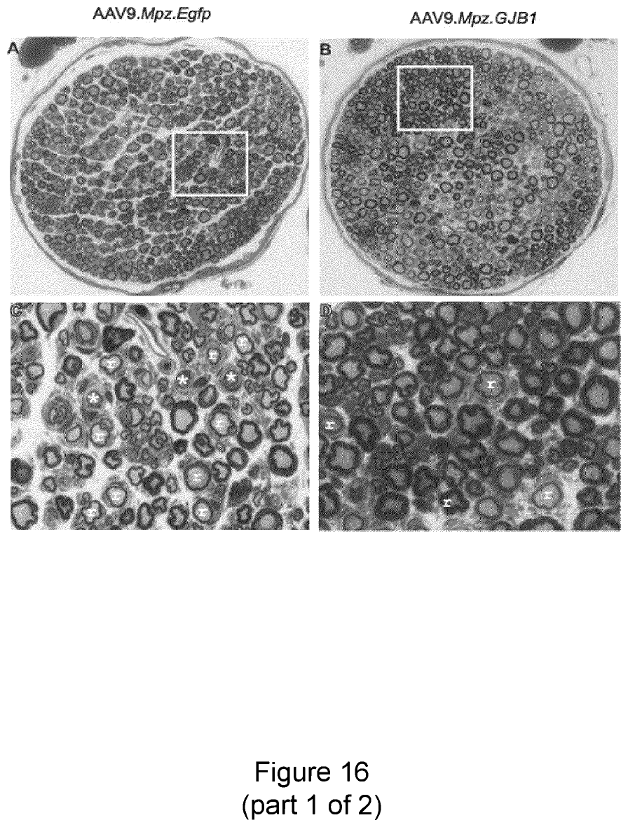

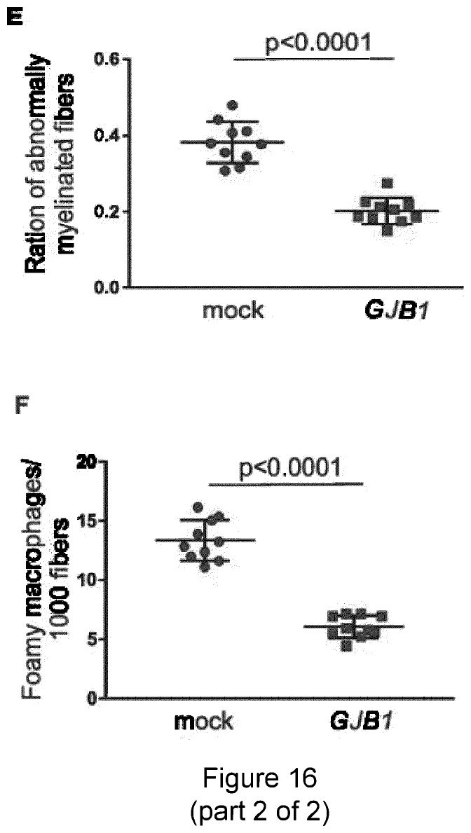

Morphological studies in peripheral nerve tissues in treated versus mock-treated 6-month old Cx32 KO mice. Semithin sections of anterior lumbar roots (FIG. 14), mid-sciatic nerves (FIG. 15), and femoral motor nerves (FIG. 16) were examined and the ratio of abnormally myelinated fibers as well as the number of macrophages were quantified in groups of fully treated compared to mock-treated Cx32 KO mice at the age of 6 months. As shown in each of FIGS. 14, 15 and 16, fewer demyelinated (*) or remyelimated (r) fibers and fewer foamy macrphages were found in treated compared to mock treated mice. This is indicative of improved myelination and a reduction in inflammation in the treated group.

### Example 11: Development of a Humanised Therapeutic Vector to Treat CMT1X

The vectors described in Example 1 are controlled by the rat Mpz promoter. In order to humanize this construct and make it more suitable for clinical applications, the inventors have also cloned a human-Mpz-GJB1 construct (SEQ ID NO: 17) using a human hP0 promoter (SEQ ID NO: 18) that can be used for preclinical dose-response testing and non-human primate (NHP) toxicity and biodistribution studies. Human P0 sequence was PCR amplified from genomic DNA using primers to introduce KpnI and AgeI restriction enzymes. The primers were: KpnhP0-F-5′-AGGGGTACCGCCTGGCATAAAC-3′ (SEQ ID NO. 25) and AgehP0-R-5′-AATTTACCGGTGCTGGGGCAG-3′ (SEQ ID NO. 26). After ligation of hP0, Cx32 ORF was cut from a pre-existing construct using BamHI and XhoI. Cx32 was ligated in the AAV transfer construct and correct assembly of the expression cassette was confirmed by restriction digest mapping and direct sequencing.

A humanized mock vector plasmid (human-Mpz-Egfp) has also been generated for use as a control (SEQ ID NO: 19).

### Example 12: Development of and Expression Analysis of a Therapeutic Vector to Treat CMT4C

A mini-Mpz-SH3TC2.myc construct similar to those described in Example 9 utilising the mini Mpz rat derived promoter of SEQ ID NO. 5, above was developed using the SH3TC2 gene insert, and with further modifications in the ITR-ITR segment (including removal of WPRE and replacement of polyA with a minimal synthetic polyA) (68, 69) to remain within the approximate 4700 bp limit to allow for efficient packaging into the AAV9. The sequence of this therapeutic vector is shown in SEQ ID NO: 20.

Expression analysis of this novel therapeutic vector (mini-Mpz-SH3TC2.myc) was conducted in groups of CMT4C model mice. These results complement the development of the minimal Mpz promoter vector driving reporter gene expression described in Example 9 above.

The novel AAV-miniMpz-SH3TC2.myc construct was produced and packaged into the AAV9 serotype achieving titers of 5×10¹² vg/ml. The vector (total of 1×10¹¹ vg in a volume of 20 μl) was delivered by lumbar intrathecal injection into 5-month old Sh3tc2−/− mice (n=5), and expression was examined 5 weeks after injection in fixed lumbar spinal root and bilateral sciatic nerve sections.

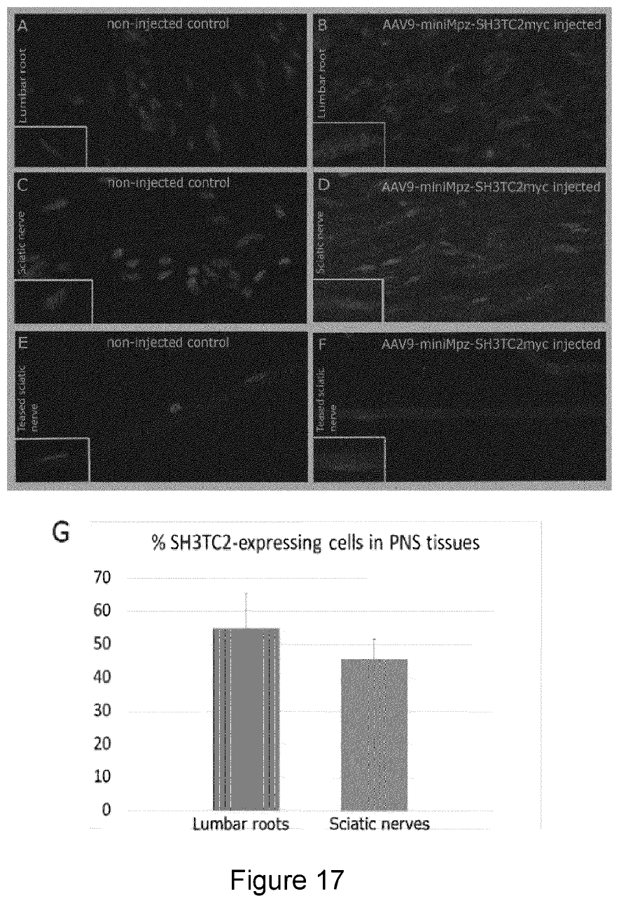

Expression of SH3TC2 was detected in a high percentage of myelinating Schwann cells throughout the PNS including roots and sciatic nerves, in a characteristic perinuclear granular appearance, and occasionally along the entire length of the Schwann cell (FIGS. 17A-F). Quantification of the percentage of SH3TC2-immunoreactive Schwann cells showed an average of 54.67% expression rate in lumbar roots and 45.39% in sciatic nerves (FIG. 17G).

These results indicate that the construct achieved a good level of expression in myelinating Schwann cells throughout the PNS.

### Example 13: Development of a Humanised Therapeutic Vector to Treat CMT4C

The mini-Mpz-SH3TC2.myc (SEQ ID NO: 20) construct (as described in Example 12 above) that is well suited for preclinical testing (due to inclusion of the minimal version of rat Mpz promoter and myc tag on SH3TC2 to facilitate preclinical expression analysis) has been modified in order to be more suitable for clinical application (SEQ ID NO: 21).

The myc tag has been removed, and the minimal version of the rat promoter has been replaced by the corresponding sequence of the minimal human Mpz promoter (SEQ ID NO: 22). This vector can be used for final preclinical dose-response testing and NHP toxicity and biodistribution studies before proceeding to clinical applications. A humanized mock vector plasmid (human-miniMpz-Egfp) has also been generated (SEQ ID NO: 23).

### REFERENCES

1. Baets J, De Jonghe P, Timmerman V. Recent advances in Charcot-Marie-Tooth disease. Curr Opin Neurol. 2014; 27(5):532-40.
2. Kleopa K A, Scherer S S. Inherited Neuropathies. Neurol Clinics North America. 2002; 20:679-709.
3. Kleopa K A, Kagiava A, Sargiannidou I. Gene Therapy for CMT Inherited Neuropathy. In: Duan D, Mendell J (eds): Muscle Gene Therapy. 2019; https://doi.org/10.1007/978-3-030-03095-7_35 (Springer, Cham).
4. Bergoffen J, Scherer S S, Wang S, Oronzi-Scott M, Bone L, Paul D L, et al. Connexin mutations in X-linked Charcot-Marie-Tooth disease. Science. 1993; 262:2039-42.
5. Kleopa K A, Scherer S S. Molecular genetics of X-linked Charcot-Marie-Tooth disease. Neuromolecular Med. 2006; 8:107-22.
6. Hahn A F, Brown W F, Koopman W J, Feasby T E. X-linked dominant hereditary motor and sensory neuropathy. Brain. 1990; 113:1511-25.
7. Birouk N, Le Guern E, Maisonobe T, Rouger H, Gouider R, Gugenheim M, et al. X-linked Charcot-Marie-Tooth disease with connexin 32 mutations—clinical and electrophysiological study. Neurology. 1998; 50:1074-82.
8. Shy M E, Siskind C, Swan E R, Krajewski K M, Doherty T, Fuerst D R, et al. CMT1X phenotypes represent loss of GJB1 gene function. Neurology. 2007; 68:849-55.
9. Dubourg O, Tardieu S, Birouk N, Gouider R, Léger J M, Maisonobe T, et al. Clinical, electrophysiological and molecular genetic characteristics of 93 patients with X-linked Charcot-Marie-Tooth disease. Brain. 2001; 124:1958-67.
10. Liang G S L, de Miguel M, Gomez-Hernandez J M, Glass J D, Scherer S S, Mintz M, et al. Severe neuropathy with leaky connexin32 hemichannels. Ann Neurol. 2005; 57:749-54.
11. Al-Mateen M, Craig A K, Chance P F. The Central Nervous System Phenotype of X-Linked Charcot-Marie-Tooth Disease: A Transient Disorder of Children and Young Adults. J Child Neurol. 2014; 29:342-8
12. Hahn A F, Ainsworth P J, Bolton C F, Bilbao J M, Vallat J-M. Pathological findings in the X-linked form of Charcot-Marie-Tooth disease: a morphometric and ultrastructural analysis. Acta Neuropathol. 2001; 101:129-39.
13. Hattori N, Yamamoto M, Yoshihara T, Koike H, Nakagawa M, Yoshikawa H, et al. Demyelinating and axonal features of Charcot-Marie-Tooth disease with mutations of myelin-related proteins (PMP22, MPZ and Cx32): a clinicopathological study of 205 Japanese patients. Brain. 2003; 126:134-51.
14. Kleopa K A, Zamba-Papanicolaou E, Alevra X, Nicolaou P, Georgiou D-M, Hadjisavvas A, et al. Phenotypic and cellular expression of two novel connexin32 mutations causing CMT1X. Neurology. 2006; 66:396-402.
15. Omori Y, Mesnil M, Yamasaki H. Connexin 32 mutations from X-linked Charcot-Marie-Tooth disease patients: functional defects and dominant negative effects. Mol Biol Cell. 1996; 7(6):907-16.
16. Yoshimura T, Satake M, Ohnishi A, Tsutsumi Y, Fujikura Y. Mutations of connexin32 in Charcot-Marie-Tooth disease type X interfere with cell-to-cell communication but not cell proliferation and myelin-specific gene expression. J Neurosci Res. 1998; 51(2):154-61.
17. Yum S W, Kleopa K A, Shumas S, Scherer S S. Diverse trafficking abnormalities of Connexin32 mutants causing CMTX. Neurobiol Dis. 2002; 11:43-52.
18. Deschênes SM, Walcott J L, Wexler T L, Scherer S S, Fischbeck K H. Altered trafficking of mutant connexin32. J Neurosci. 1997; 17:9077-84.
19. Oh S, Ri Y, Bennett M V L, Trexler E B, Verselis V K, Bargiello T A. Changes in permeability caused by connexin 32 mutations underlie X-linked Charcot-Marie-Tooth disease. Neuron. 1997; 19(4):927-38.
20. Martin P E M, Mambetisaeva E T, Archer D A, George C H, Evans W H. Analysis of gap junctions assembly using mutated connexins detected in Charcot-Marie-Tooth X-linked disease. J Neurochem. 2000; 74:711-20.
21. Kleopa K A, Yum S W, Scherer S S. Cellular mechanisms of connexin32 mutations associated with CNS manifestations. J Neurosci Res. 2002; 68:522-34.
22. Sargiannidou I, Vavlitou N, Aristodemou S, Hadjisavvas A, Kyriacou K, Scherer S S, et al. Connexin32 mutations cause loss of function in Schwann cells and oligodendrocytes leading to PNS and CNS myelination defects. J Neurosci. 2009; 29:4748-61.
23. Sargiannidou I, Kim G H, Kyriakoudi S, Eun B L, Kleopa K A. A start codon CMT1X mutation associated with transient encephalomyelitis causes complete loss of Cx32. Neurogenetics. 2015; 16(3):193-200.
24. Anzini P, Neuberg DH-H, Schachner M, Nelles E, Willecke K, Zielasek J, et al. Structural abnormalities and deficient maintenance of peripheral nerve myelin in mice lacking the gap junction protein connexin32. J Neurosci. 1997; 17:4545-61.
25. Scherer S S, Xu Y-T, Nelles E, Fischbeck K, Willecke K, Bone L J. Connexin32-null mice develop a demyelinating peripheral neuropathy. Glia. 1998; 24:8-20.
26. Scherer S S, Xu Y T, Messing A, Willecke K, Fischbeck K H, Jeng L J. Transgenic expression of human connexin32 in myelinating Schwann cells prevents demyelination in connexin32-null mice. J Neurosci. 2005; 25:1550-9.
27. Abel A, Bone L J, Messing A, Scherer S S, Fischbeck K F. Studies in transgenic mice indicate a loss of connexin32 function in X-linked Charcot-Marie-Tooth disease. J Neuropathol Exp Neurol. 1999; 58:702-10.
28. Jeng L J, Balice-Gordon R J, Messing A, Fischbeck K H, Scherer S S. The effects of a dominant connexin32 mutant in myelinating Schwann cells. Mol Cell Neurosci. 2006; 32:283-98.
29. Kagiava A, Karaiskos C, Richter J, Tryfonos C, Lapathitis G, Sargiannidou I, et al. Intrathecal gene therapy in mouse models expressing CMT1X mutations. Hum Mol Genet. 2018; 27(8):1460-73.
30. Huang Y, Sirkowski E E, Stickney J T, Scherer S S. Prenylation-defective human connexin32 mutants are normally localized and function equivalently to wild-type connexin32 in myelinating Schwann cells. J Neurosci. 2005; 25:7111-20.
31. Hahn A F, Ainsworth P J, Naus C C G, Mao J, Bolton C F. Clinical and pathological observations in men lacking the gap junction protein connexin 32. Muscle Nerve. 2000:S39-S48.
32. Sargiannidou I, Kagiava A, Bashiardes S, Richter J, Christodoulou C, Scherer S S, et al. Intraneural GJB1 gene delivery improves nerve pathology in a model of CMT1X. Ann Neurol. 2015; 78:303-16.
33. Kagiava A, Sargiannidou I, Theophilidis G, Karaiskos C, Richter J, Bashiardes S, et al. Intrathecal gene therapy rescues a model of demyelinating peripheral neuropathy. Proc Natl Acad Sci USA. 2016; 113(17):E2421-9. doi: 10.1073/pnas.1522202113.
34. Kyriakoudi S, Sargiannidou I, Kagiava A, Olympiou M, Kleopa K A. Golgi-retained Cx32 mutants interfere with gene addition therapy for CMT1X. Hum Mol Genet. 2017; 26(9):1622-33.
35. Fridman V, Bundy B, Reilly M M, Pareyson D, Bacon C, Burns J, et al. CMT subtypes and disease burden in patients enrolled in the Inherited Neuropathies Consortium natural history study: a cross-sectional analysis. J Neurol Neurosurg Psychiatry. 2015; 86(8):873-8.
36. Kessali M, Zemmouri R, Guilbot A, Maisonobe T, Brice A, LeGuern E, et al. A clinical, electrophysiologic, neuropathologic, and genetic study of two large Algerian families with an autosomal recessive demyelinating form of Charcot-Marie-Tooth disease. Neurology. 1997; 48(4):867-73.
37. Gabreels-Festen A, van Beersum S, Eshuis L, LeGuern E, Gabreels F, van Engelen B, et al. Study on the gene and phenotypic characterisation of autosomal recessive demyelinating motor and sensory neuropathy (Charcot-Marie-Tooth disease) with a gene locus on chromosome 5q23-q33. J Neurol Neurosurg Psychiatry. 1999; 66(5):569-74.
38. Azzedine H, Ravise N, Verny C, Gabreels-Festen A, Lammens M, Grid D, et al. Spine deformities in Charcot-Marie-Tooth 4C caused by SH3TC2 gene mutations. Neurology. 2006; 67(4):602-6.
39. Gooding R, Colomer J, King R, Angelicheva D, Marns L, Parman Y, et al. A novel Gypsy founder mutation, p.Arg1109X in the CMT4C gene, causes variable peripheral neuropathy phenotypes. J Med Genet. 2005; 42(12):e69.
40. Colomer J, Gooding R, Angelicheva D, King R H, Guillen-Navarro E, Parman Y, et al. Clinical spectrum of CMT4C disease in patients homozygous for the p.Arg1109X mutation in SH3TC2. Neuromuscul Disord. 2006; 16(7):449-53.
41. Varley T L, Bourque P R, Baker S K. Phenotypic variability of CMT4C in a French-Canadian kindred. Muscle Nerve. 2015.
42. Senderek J, Bergmann C, Stendel C, Kirfel J, Verpoorten N, De Jonghe P, et al. Mutations in a Gene Encoding a Novel SH3/TPR Domain Protein Cause Autosomal Recessive Charcot-Marie-Tooth Type 4C Neuropathy. Am J Hum Genet. 2003; 73:1106-19.
43. LeGuern E, Guilbot A, Kessali M, Ravise N, Tassin J, Maisonobe T, et al. Homozygosity mapping of an autosomal recessive form of demyelinating Charcot-Marie-Tooth disease to chromosome 5q23-q33. Hum Mol Genet. 1996; 5(10):1685-8.
44. Lassuthova P, Mazanec R, Vondracek P, Siskova D, Haberlova J, Sabova J, et al. High frequency of SH3TC2 mutations in Czech HMSN I patients. Clin Genet. 2011; 80(4):334-45.
45. Lupo V, Galindo M I, Martinez-Rubio D, Sevilla T, Vilchez J J, Palau F, et al. Missense mutations in the SH3TC2 protein causing Charcot-Marie-Tooth disease type 4C affect its localization in the plasma membrane and endocytic pathway. Hum Mol Genet. 2009; 18(23):4603-14.
46. Arnaud E, Zenker J, de Preux Charles A S, Stendel C, Roos A, Medard J J, et al. SH3TC2/KIAA1985 protein is required for proper myelination and the integrity of the node of Ranvier in the peripheral nervous system. Proc Natl Acad Sci USA. 2009; 106(41):17528-33.
47. Gouttenoire E A, Lupo V, Calpena E, Bartesaghi L, Schupfer F, Medard J J, et al. Sh3tc2 deficiency affects neuregulin-1/ErbB signaling. Glia. 2013; 61(7):1041-51.
48. Zoupi L, Savvaki M, Karagogeos D. Axons and myelinating glia: An intimate contact. IUBMB Life. 2011; 63(9):730-5.
49. Schiza N, Georgiou E, Kagiava A, Médard J-J, Richter J, Tryfonos C, et al. Gene replacement therapy in a model of Charcot-Marie-Tooth 4C neuropathy. Brain. 2019; 142(5):1227-1241.
50. Tanguy Y, Biferi M G, Besse A, Astord S, Cohen-Tannoudji M, Marais T, et al. Systemic AAVrh10 provides higher transgene expression than AAV9 in the brain and the spinal cord of neonatal mice. Front Mol Neurosci. 2015; 8:36.
51. Foust K D, Nurre E, Montgomery C L, Hernandez A, Chan C M, Kaspar B K. Intravascular A AV9 preferentially targets neonatal neurons and adult astrocytes. Nat Biotechnol. 2009; 27(1):59-65.
52. Gurda B L, De Guilhem De Lataillade A, Bell P, Zhu Y, Yu H, Wang P, et al. Evaluation of AAV-mediated Gene Therapy for Central Nervous System Disease in Canine Mucopolysaccharidosis VII. Mol Ther. 2016; 24(2):206-16.
53. Calcedo R, Wilson J M. Humoral Immune Response to AAV. Front Immunol. 2013; 4:341.
54. Jackson K L, Dayton R D, Klein R L. AAV9 supports wide-scale transduction of the CNS and TDP-43 disease modeling in adult rats. Mol Ther Methods Clin Dev. 2015; 2:15036.
55. Meyer K, Ferraiuolo L, Schmelzer L, Braun L, McGovern V, Likhite S, et al. Improving single injection CSF delivery of AAV9-mediated gene therapy for SMA: a dose-response study in mice and nonhuman primates. Mol Ther. 2015; 23(3):477-87.
56. Jang S W, Svaren J. Induction of myelin protein zero by early growth response 2 through upstream and intragenic elements. J Biol Chem. 2009; 284(30):20111-20.
57. von Jonquieres G, Mersmann N, Klugmann C B, Harasta A E, Lutz B, Teahan O, et al. Glial promoter selectivity following AAV-delivery to the immature brain. PLoS One. 2013; 8(6):e65646.
58. Georgiou E, Sidiropoulou K, Richter J, Papaneophytou C, Sargiannidou I, Kagiava A, et al. Gene therapy targeting oligodendrocytes provides therapeutic benefit in a leukodystrophy model. Brain. 2017; 140(3):599-616.
59. Zolotukhin S, Byrne B J, Mason E, Zolotukhin I, Potter M, Chesnut K, et al. Recombinant adeno-associated virus purification using novel methods improves infectious titer and yield. Gene Ther. 1999; 6(6):973-85.
60. Piedra J, Ontiveros M, Miravet S, Penalva C, Monfar M, Chillon M. Development of a rapid, robust, and universal picogreen-based method to titer adeno-associated vectors. Hum Gene Ther Methods. 2015; 26(1):35-42.
61. Savvaki M, Panagiotaropoulos T, Stamatakis A, Sargiannidou I, Karatzioula P, Watanabe K, et al. Impairment of learning and memory in TAG-1 deficient mice associated with shorter CNS internodes and disrupted juxtaparanodes. Mol Cell Neurosci. 2008; 39:478-90.
62. Zielasek J, Martini R, Toyka K V. Functional abnormalities in P0-deficient mice resemble human hereditary neuropathies linked to P0 gene mutations. Muscle Nerve. 1996; 19(8):946-52.
63. Vavlitou N, Sargiannidou I, Markoullis K, Kyriacou K, Scherer S S, Kleopa K A. Axonal pathology precedes demyelination in a mouse model of X-linked demyelinating/type I Charcot-Marie Tooth neuropathy. J Neuropathol Exp Neurol. 2010; 69:945-58.
64. Kobsar I, Berghoff M, Samsam M, Wessig C, Maurer M, Toyka K V, et al. Preserved myelin integrity and reduced axonopathy in connexin32-deficient mice lacking the recombination activating gene-1. Brain. 2003; 126:804-13.
65. Groh J, Heinl K, Kohl B, Wessig C, Greeske J, Fischer S, et al. Attenuation of MCP-1/CCL2 expression ameliorates neuropathy in a mouse model for Charcot-Marie-Tooth 1X. Hum Mol Genet. 2010; 19:3530-43.
66. Shevtsova, Z., et al. (2005). “Promoters and serotypes: targeting of adeno-associated virus vectors for gene transfer in the rat central nervous system in vitro and in vivo.” Exp Physiol; 90(1): 53-59
67. von Jonquieres, G., et al. (2016). “Recombinant Human Myelin-Associated Glycoprotein Promoter Drives Selective AAV-Mediated Transgene Expression in Oligodendrocytes.” Front Mol Neurosci; 9: 13
68. Levitt, N., Briggs, D., Gil, A., and Proudfoot, N.J. (1989). Definition of an efficient synthetic poly(A) site. Genes Dev. 3, 1019-1025.
69. Bailey R M, Armao D, Nagabhushan Kalburgi S, Gray S J. Development of Intrathecal AAV9 Gene Therapy for Giant Axonal Neuropathy. Mol Ther Methods Clin Dev. 2018 Feb. 15; 9:160-171.

Embodiments of the invention will now be described in the following numbered paragraphs:
1. A viral vector for use in treating or preventing a disease associated with Schwann cells in a subject in need thereof, wherein the viral vector comprises a first nucleic acid sequence that can be transcribed into a first polynucleotide, and wherein the viral vector is an AAV vector.
2. The viral vector for use of paragraph 1, wherein the expression of the first polynucleotide is under the control of a Schwann cell specific promoter, optionally a myelin specific promoter.
3. The viral vector for use of paragraphs 1 or 2, wherein the expression of the first polynucleotide is under the control of the full-length myelin protein zero (Mpz) promoter, wherein the full-length promoter is a full-length rat or human myelin protein zero promoter.
4. The viral vector for use of paragraphs 1-3 wherein the expression of the first polynucleotide is under the control of a promoter that is between 100 bp and 1100 bp in length, optionally wherein the promoter ranges from 200 bp to 900 bp in length, 300 bp to 800 bp in length, 400 bp to 700 bp in length, optionally wherein the promoter ranges from 500 bp to 600 bp in length, optionally wherein the promoter is 410 bp in length.
5. The viral vector for use of paragraph 4 wherein the promoter is a full-length or a minimal myelin specific promoter, optionally a minimal myelin protein zero (Mpz) promoter, optionally wherein the promoter has a sequence with at least 75% sequence homology or sequence identity with SEQ ID NO. 5 or SEQ ID NO. 22, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 5 or SEQ ID NO. 22.
6. The viral vector for use of any one of the preceding paragraphs, wherein the vector has the ability to transduce Schwann cells.
7. The viral vector for use of any one of the preceding paragraphs, wherein the vector does not integrate into the genome of the host cell.
8. The viral vector for use of any one of the preceding paragraphs, wherein the AAV vector is selected from the group comprising: AAV9 and AAVrh10.
9. The viral vector for use of paragraph 8, wherein the AAV vector is an AAV9.
10. The viral vector for use of any one of the preceding paragraphs wherein the first polynucleotide encodes and is translated into a first polypeptide or protein.
11. The viral vector for use of paragraph 10 wherein the first nucleic acid comprises:
a) a wild-type or therapeutically beneficial sequence of a neuropathy-associated gene, optionally selected from the group comprising or consisting of any one of the following 20 genes: gap junction beta 1 (GJB1); SH3 domain and tetratricopeptide repeats 2 (SH3TC2); peripheral myelin protein 22 (PMP22); myelin protein zero (MPZ); early growth response 2 (EGR2); ganglioside induced differentiation associated protein 1 (GDAP1); N-Myc downstream regulated 1 (NDRG1) or other genes associated with demyelinating neuropathy and Schwann cell dysfunction; or
b) a sequence with at least 75% sequence homology or sequence identity, optionally at least 80%, or 82%, or 84%, or 86%, or 88%, or 90%, or 92%, or 94%, or 96%, or 97%, or 98%, or 99%, or 100% sequence identity or sequence homology to a wild-type sequence of a neuropathy-associated gene, for example one of the following genes: gap junction beta 1 (GJB1); SH3 domain and tetratricopeptide repeats 2 (SH3TC2); peripheral myelin protein 22 (PMP22); myelin protein zero (MPZ); early growth response 2 (EGR2); ganglioside induced differentiation associated protein 1 (GDAP1); N-Myc downstream regulated 1 (NDRG1) or other genes associated with demyelinating neuropathy and Schwann cell dysfunction;
optionally wherein the first nucleic acid comprises a sequence with at least 75% sequence homology or sequence identity with SEQ ID NOs. 6-12, optionally at least 80%, or 82%, or 84%, or 86%, or 88%, or 90%, or 92%, or 94%, or 96%, or 97%, or 98%, or 99%, or 100% sequence identity or sequence homology to SEQ ID NOs. 6-12.
12. The viral vector for use of paragraphs 10 or 11 wherein the first nucleic acid comprises the wild-type form of the open reading frame (ORF) or cDNA that is transcribed into a first polynucleotide encoding one or more polypeptides, optionally selected from the group comprising or consisting of: connexin-32 (Cx32); SH3 domain and tetratricopeptide repeats 2 (SH3TC2); peripheral myelin protein 22 (PMP22); myelin protein zero (MPZ); early growth response 2 (EGR2); ganglioside induced differentiation associated protein 1 (GDAP1); N-Myc downstream regulated 1 (NDRG1).
13. The viral vector for use of paragraphs 10-11 wherein the first nucleic acid comprises the wild-type open reading frame (ORF) of the gap junction beta 1 (GJB1) gene.
14. The viral vector for use of any one of paragraphs 1-13 wherein the vector is capable of driving expression from the first polynucleotide, optionally driving expression of a first polypeptide, optionally wherein the first polypeptide is connexin 32 (Cx32) protein, optionally wild-type Cx32.
15. The viral vector for use of any one of paragraphs 1-10 wherein the first polynucleotide encodes one or more of the following: a trophic factor (e.g. BDNF, GDNF, NT-3, VEGF), a regenerative factor (e.g. Angiogenin, Oct-6, Egr2, Sox-10), a growth factor (e.g. IGF).
16. The viral vector for use of any one of the preceding paragraphs, wherein administration of the viral vector results in an expression of a first protein from the first polynucleotide that leads to improved functioning of Schwann cells and/or increased formation of myelin sheath.
17. The viral vector for use of paragraphs 1-9 wherein the first polynucleotide does not encode a polypeptide, optionally wherein the first polynucleotide is a non-coding RNA.
18. The viral vector for use of paragraph 17 wherein the non-coding RNA is a short hairpin RNA (shRNA); microRNA (miRNA); guide RNA (gRNA).
19. The viral vector for use of any one of paragraphs 17 or 18 wherein when the viral vector is in a target organism, expression of the non-coding RNA causes a reduction in expression of a target polynucleotide, optionally wherein the target polynucleotide is a gene located in a target organism, optionally located in a cell in a target organism.
20. The viral vector for use of paragraph 19 wherein expression or overexpression of the target polynucleotide in a target organism is considered to be associated with a disease associated with Schwann cells, optionally wherein the disease is a dominant demyelinating neuropathy (CMT1), optionally wherein the target polynucleotide is a mutated allele of myelin protein zero (Mpz/P0) and the disease associated with Schwann cells is CMT1B, or wherein the target polynucleotide is another dominant gene associated with CMT1.
21. The viral vector for use of any one of paragraphs 17-20, wherein administration of the viral vector results in improved functioning of Schwann cells and/or increased formation of myelin sheath.
22. The viral vector for use of any one of the preceding paragraphs wherein the disease associated with Schwann cells causes destruction and/or reduced formation of myelin sheath by Schwann cells.
23. The viral vector for use of any one of the preceding paragraphs, where the disease is selected from the group consisting of: Charcot-Marie-Tooth disease (CMT); hereditary neuropathy with liability to pressure palsies (HNPP); diabetic and other toxic peripheral neuropathies; motor neuron disease (MND).
24. The viral vector for use of any one of the preceding paragraphs, wherein the disease is Charcot-Marie-Tooth disease (CMT).
25. The viral vector for use of paragraph 24, wherein the disease is selected from: Charcot-Marie-Tooth type 1X (CMT1X); Charcot-Marie-Tooth types 1A-1 F (CMT1A-1 F); Charcot-Marie-Tooth types 4A-4H (CMT4A-4H).
26. The viral vector for use of paragraph 25, wherein the disease is Charcot-Marie-Tooth type 1X (CMT1X).
27. The viral vector for use of paragraph 25, wherein the disease is Charcot-Marie-Tooth type 4C (CMT4C).
28. The viral vector for use of paragraphs 16 or 21, wherein the improved function results from increased formation of myelin sheath by Schwann cells when compared to the formation of myelin sheath by Schwann cells in the subject prior to treatment.
29. The viral vector for use of paragraph 28, wherein the increased formation of myelin sheath by Schwann cells leads to an improvement in any one or more of the following parameters:
a) muscle strength;
b) sciatic nerve conduction velocity; and/or
c) response of blood biomarkers, when compared to the subject prior to treatment.
30. The viral vector for use of paragraph 28 or 29, wherein the improved formation of myelin sheath by Schwann cells leads to improved myelination of the peripheral nerves.
31. The viral vector for use of any one of the preceding paragraphs, wherein the AAV is administered to the subject by intrathecal injection or intravenous injection, preferably wherein the AAV is administered by intrathecal injection.
32. The viral vector for use of paragraph 31 wherein the AAV is administered by one of the following routes: lumbar intrathecal injection; thoracic intrathecal injection; cervical intrathecal injection.
33. The viral vector for use of paragraph 32, wherein the viral vector is administered by lumbar intrathecal injection.
34. The viral vector for use of paragraphs 31-33, wherein the AAV is administered by a single intrathecal injection.
35. The viral vector for use of any one of the preceding paragraphs, wherein the subject in need thereof is a human subject.
36. A viral vector as defined by any of the preceding paragraphs.
37. A cell that has been transduced with a viral vector as defined by any of the preceding paragraphs, optionally wherein the cell is a Schwann cell.
38. A minimal myelin specific promoter, wherein the minimal myelin specific promoter has a sequence homology with at least 75% sequence homology or sequence identity with SEQ ID NO. 5 or SEQ ID NO. 22, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 5 or SEQ ID NO. 22.
39. A minimal myelin specific promoter comprising or consisting of the sequence of SEQ ID NO. 5 or SEQ ID NO. 22.
40. A polynucleotide construct comprising a first nucleic acid sequence that is a Schwann cell specific promoter, optionally a myelin specific promoter, optionally comprising the myelin protein zero (Mpz) promoter or a minimal myelin specific promoter as defined in paragraphs 38 or 39, operably linked to a second nucleic acid sequence, wherein the second nucleic acid is transcribed into a first polynucleotide and wherein the second nucleic acid sequence: a) is the open reading frame or cDNA or other elements of a gene; or b) is transcribed into a non-coding RNA.
41. A viral vector comprising the minimal myelin specific promoter according to any of paragraphs 38 or 39 or the polynucleotide construct of paragraph 40.
42. The viral vector for use of any one of paragraphs 1-35 or the viral vector of paragraphs 36 or 41, wherein the vector has the ability to transduce Schwann cells.
43. The viral vector for use of any one of the preceding paragraphs, wherein the vector does not integrate into the genome of the host cell.
44. A viral vector according to one any one of paragraphs 42 or 43 comprising:
a) an AAV, optionally wherein the AAV vector is an AAV9;
b) an AAV-Mpz.Egfp vector comprising an AAV9 vector, the myelin protein zero (Mpz) promoter and the EGFP reporter gene;
c) an AAV9-Mpz-GJB1 vector comprising an AAV9 vector, the myelin protein zero (Mpz) promoter and the open reading frame (ORF) of the gap junction beta 1 (GJB1) gene;
d) an AAV9-miniMpz.Egfp vector comprising an AAV9 vector, the minimal myelin protein zero (miniMpz) promoter and the EGFP reporter gene;
e) an AAV9-human Mpz-GJB1 vector comprising an AAV9 vector, the full-length human myelin protein zero (hP0) promoter and the open reading frame (ORF) of the gap junction beta 1 (GJB1) gene (SEQ ID NO. 17);
f) an AAV9-human Mpz-Egfp vector comprising an AAV9 vector, the full-length human myelin protein zero (hP0) promoter and the EGFP reporter gene (SEQ ID NO. 19);
g) an AAV9-miniMpz-SH3TC2.myc.ITR vector comprising an AAV9 vector, a minimal rat myelin protein zero (Mpz) promoter and the open reading frame (ORF) of the SH3TC2 gene (SEQ ID NO. 20);
h) an AAV9-human-miniMpz-SH3TC2 vector comprising an AAV9 vector, a human minimal myelin protein zero (hP0) promoter and the open reading frame (ORF) of the SH3TC2 gene (SEQ ID NO. 21); or
i) an AAV9-human-miniMpz-Egfp vector comprising an AAV9 vector, a human minimal myelin protein zero (hP0) promoter and the EGFP reporter gene (SEQ ID NO. 23).
45. A pharmaceutical composition comprising the viral vector of any one of the preceding paragraphs.
46. The pharmaceutical composition of paragraph 45, wherein the composition comprises an appropriate amount of the viral vector and further comprises a pharmaceutically acceptable carrier and/or excipient.
45. Use of a viral vector according to any of the preceding paragraphs in a method of manufacture of a medicament for the treatment or prevention of a disease associated with Schwann cells, optionally wherein the disease causes destruction and/or reduced formation of myelin sheath by Schwann cells, optionally wherein the disease is Charcot-Marie-Tooth disease.
46. A viral vector or polynucleotide construct according to any of the preceding paragraphs for use in a CRISPR/Cas9 system wherein the viral vector or polynucleotide comprises any one or more of:
a) a polynucleotide encoding a single guide RNA (sgRNA) targeting a gene of interest;
b) a polynucleotide encoding a Cas9 polypeptide;
c) a polynucleotide encoding a polypeptide of interest.
47. A viral vector according to any of the preceding paragraphs, for use in a method of labelling Schwann cells, for example labelling with fluorescent protein, for example green fluorescent protein (GFP) or enhanced green fluorescent protein (EGFP), or another non-fluorescent reporter, optionally wherein the labelling of Schwann cells can be used in a method of diagnosing a disease associated with Schwann cells.
48. A viral vector according to any one of paragraphs 1-43, for use in a method wherein Schwann cells are induced to differentiate into an alternative cell type (for example oligodendrocytes, astrocytes or neurons).
49. A viral vector according to any one of paragraphs 1-43, for use in a method of stimulating Schwann cells to support regeneration in a subject in need thereof, optionally after an injury or trauma.
50. A kit for use preventing or treating a disease associated with Schwann cells, labelling Schwann cells or regenerating Schwann cells wherein the kit comprises one or more of:
a) a viral vector as defined in any of the preceding paragraphs;
b) a polynucleotide construct as defined by paragraph 40;
c) a viral vector;
d) a viral vector comprising the polynucleotide construct as defined by paragraph 40;
e) a pharmaceutically acceptable carrier and/or excipient;
f) a single-use syringe, for example a single-use syringe suitable for intrathecal lumbar injection;
g) instructions for use.
51. A kit according to paragraph 50, wherein the kit comprises more than one viral vector as defined by any one of the preceding paragraphs, optionally wherein the kit comprises 2, 3, 4, 5, 6, 7, 8, 9 or 10 different viral vectors as defined by any one of the preceding paragraphs.
52. A viral vector for use in treating or preventing a disease associated with Schwann cells in a subject in need thereof, wherein the viral vector comprises a first nucleic acid sequence that can be transcribed into a first polynucleotide, and wherein expression of said first polynucleotide is under the control of a minimal myelin specific promoter, optionally comprising or consisting of the sequence defined in SEQ ID NO. 5 or SEQ ID NO. 22, optionally wherein the viral vector is an AAV vector.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the following claims.

1. A method of treating or preventing a disease associated with Schwann cells in a subject in need thereof, wherein the method comprises administering to said subject a viral vector that comprises a first nucleic acid sequence that can be transcribed into a first polynucleotide, and wherein the viral vector is an adeno-associated virus (AAV) vector.

2. The method of claim 1, wherein the expression of the first polynucleotide is under the control of a Schwann cell specific promoter, optionally a myelin specific promoter, optionally wherein the myelin specific promoter is:
a) a full-length myelin protein zero (Mpz) promoter, optionally wherein the full-length Mpz promoter is a full-length rat or full-length human Mpz promoter;
b) a full-length myelin protein zero (Mpz) promoter, wherein the promoter has a sequence with at least 75% sequence homology or sequence identity with SEQ ID NO. 4 or SEQ ID NO. 18, optionally at least 80%, or 82%, or 84%, or 86%, or 88%, or 90%, or 92%, or 94%, or 96%, or 97%, or 98%, or 99%, or 100% sequence identity or sequence homology to SEQ ID NO. 4 or SEQ ID NO. 18;
c) a minimal myelin protein zero (miniMpz) promoter, optionally wherein the minimal Mpz promoter is a rat or human minimal Mpz promoter; or
d) a minimal myelin protein zero (miniMpz) promoter, wherein the promoter has a sequence with at least 75% sequence homology or sequence identity with SEQ ID NO. 5 or SEQ ID NO. 22.

3. The method of claim 1 wherein the expression of the first polynucleotide is under the control of a promoter, optionally a Schwann cell specific promoter, that:
a) is between 100 bp and 1100 bp in length, optionally wherein the promoter ranges from 200 bp to 900 bp in length, 300 bp to 800 bp in length, 400 bp to 700 bp in length, optionally wherein the promoter ranges from 500 bp to 600 bp in length, optionally wherein the promoter is 410 bp in length; and/or
b) is less than 1100 bp, 1000 bp, 900 bp, 800 bp, 700 bp, 600 bp, 500 bp, 450 bp, 400 bp, 350 bp, 300 bp, 250 bp, 200 bp, 150 bp, 100 bp in length.

4. The method of claim 1, wherein the AAV vector is selected from the group comprising: AAV9 and AAVrh10.

5. The method of claim 1 wherein the first nucleic acid sequence encodes and is translated into a first polypeptide or protein,
optionally wherein the first nucleic acid comprises:
a) a wild-type or therapeutically beneficial sequence of a neuropathy-associated gene, optionally selected from the group comprising or consisting of any one of the following genes: gap junction beta 1 (GJB1); SH3 domain and tetratricopeptide repeats 2 (SH3TC2); peripheral myelin protein 22 (PMP22); myelin protein zero (MPZ); early growth response 2 (EGR2); ganglioside induced differentiation associated protein 1 (GDAP1); and N-Myc downstream regulated 1 (NDRG1); or other genes associated with demyelinating neuropathy and Schwann cell dysfunction; or
b) a sequence with at least 75% sequence homology or sequence identity, optionally at least 80%, or 82%, or 84%, or 86%, or 88%, or 90%, or 92%, or 94%, or 96%, or 97%, or 98%, or 99%, or 100% sequence identity or sequence homology to a wild-type sequence of a neuropathy-associated gene, for example one of the following genes: gap junction beta 1 (GJB1); SH3 domain and tetratricopeptide repeats 2 (SH3TC2); peripheral myelin protein 22 (PMP22); myelin protein zero (MPZ); early growth response 2 (EGR2); ganglioside induced differentiation associated protein 1 (GDAP1); N-Myc downstream regulated 1 (NDRG1); or other genes associated with demyelinatini neuropathy and Schwann cell dysfunction;
optionally wherein the first nucleic acid sequence comprises a sequence with at least 75% sequence homology or sequence identity with SEQ ID NOs. 6-12.

6. (canceled)

7. The method of claim 1 wherein the first nucleic acid sequence comprises the wild-type form of the open reading frame (ORF) or cDNA that is transcribed into a first polynucleotide encoding one or more polypeptides, optionally selected from the group comprising or consisting of: connexin-32 (Cx32); SH3 domain and tetratricopeptide repeats 2 (SH3TC2); peripheral myelin protein 22 (PMP22); myelin protein zero (MPZ); early growth response 2 (EGR2); ganglioside induced differentiation associated protein 1 (GDAP1); and N-Myc downstream regulated 1 (NDRG1); or
wherein the vector is capable of driving expression from the first nucleic acid sequence, optionally driving expression of a first polypeptide, optionally wherein the first polypeptide is selected from the group comprising or consisting of connexin-32 (Cx32); SH3 domain and tetratricopeptide repeats 2 (SH3TC2); peripheral myelin protein 22 (PMP22); myelin protein zero (MPZ); early growth response 2 (EGR2); ganglioside induced differentiation associated protein 1 (GDAP1); N-Myc downstream regulated 1 (NDRG1).

8. (canceled)

9. (canceled)

10. The method of claim 1 wherein the first nucleic acid sequence does not encode a polypeptide.

11. The method of claim 1 wherein the non-coding RNA causes a reduction in expression of a target polynucleotide.

12. The method of claim 11 wherein expression or overexpression of the target polynucleotide in a target organism is considered to be associated with a disease associated with Schwann cells, optionally wherein the disease is a dominant demyelinating neuropathy (CMT1), optionally wherein the target polynucleotide is a mutated allele of myelin protein zero (Mpz/P0) and the disease associated with Schwann cells is CMT1B, or wherein the target polynucleotide is another dominant gene associated with CMT1.

13. The method of claim 1 wherein the disease associated with Schwann cells
is selected from the group consisting of:
Charcot-Marie-Tooth disease (CMT);
hereditary neuropathy with liability to pressure palsies (HNPP);
diabetic and other toxic peripheral neuropathies;
motor neuron disease (MND);
Charcot-Marie-Tooth type 1X (CMT1X); Charcot-Marie-Tooth type 1A;
Charcot-Marie-Tooth type 1B;
Charcot-Marie-Tooth type 1C;
Charcot-Marie-Tooth type 1D;
Charcot-Marie-Tooth type 1E;
Charcot-Marie-Tooth type 1F;
Charcot-Marie-Tooth type 4A;
Charcot-Marie-Tooth type 4B;
Charcot-Marie-Tooth type 4C;
Charcot-Marie-Tooth type 4D;
Charcot-Marie-Tooth type 4E;
Charcot-Marie-Tooth type 4F;
Charcot-Marie-Tooth type 4G; and
Charcot-Marie-Tooth type 4H.

14. The method of claim 1, wherein administration of the viral vector results in improved functioning of Schwann cells and/or increased formation of myelin sheath by Schwann cells when compared to the formation of myelin sheath by Schwann cells in the subject prior to treatment, optionally
as detected by assessment of any one or more of the following parameters:
a) muscle strength;
b) sciatic nerve conduction velocity; and/or
c) response of blood biomarkers.

15. (canceled)

16. The method of claim 1, wherein the AAV is administered to the subject by:
intrathecal injection
intravenous injections
lumbar intrathecal injection;
thoracic intrathecal injection; or
cervical intrathecal injection.

17.-20. (canceled)

21. A minimal myelin specific promoter, wherein the minimal myelin specific promoter is a minimal myelin protein zero (miniMpz), wherein the minimal myelin specific promoter has a sequence with at least 75% sequence homology or sequence identity with SEQ ID NO. 5 or SEQ ID NO. 22.

22. (canceled)

23. A polynucleotide construct comprising:
a first nucleic acid sequence that is a Schwann cell specific promoter operably linked to a second nucleic acid sequence, wherein the Schwann cell specific promoter is:
a) a full-length Mpz promoter with at least 75% sequence homology or sequence identity with SEQ ID NO. 4 or SEQ ID NO. 18; or
b) a minimal Schwann cell specific promoter as defined by claim 21,
wherein the second nucleic acid sequence is transcribed into a first polynucleotide, that a) encodes for a polypeptide; or b) is a non-coding RNA.

24. A viral vector comprising:
a) the minimal myelin specific promoter wherein the promoter has a sequence with at least 75% sequence homology or sequence identity with SEQ ID NO: 5 or SEQ ID NO: 22;
b) a full-length Mpz promoter wherein the promoter has a sequence with at least 75% sequence homology or sequence identity with SEQ ID NO. 4 or SEQ ID NO. 18; or
c) the polynucleotide construct of claim 23.

25. The viral vector of claim 24, wherein the vector is able to transduce Schwann cells, and/or
wherein the vector does not integrate into the genome of the host cell.

26. The viral vector according to claim 25 wherein the viral vector comprises:
a) an AAV vector, optionally wherein the AAV vector is an AAV9 or AAVrh10;
b) an AAV-Mpz.Egfp vector comprising an AAV9 vector, the myelin protein zero (Mpz) promoter and an EGFP reporter gene, wherein the promoter has a sequence with at least 75% sequence homology or sequence identity with SEQ ID NO. 4 or SEQ ID NO. 18;
c) an AAV9-Mpz-GJB1 vector comprising an AAV9 vector, the myelin protein zero (Mpz) promoter and an open reading frame (ORF) of the gap junction beta 1 (GJB1) gene, wherein the promoter has a sequence with at least 75% sequence homology or sequence identity with SEQ ID NO. 4 or SEQ ID NO. 18;
d) an AAV9-miniMpz.Egfp vector comprising an AAV9 vector, the minimal myelin protein zero (miniMpz) promoter and an EGFP reporter gene, wherein the miniMPZ promoter has a sequence homology with at least 75 sequence homology or sequence identity with SEQ ID NO. 5 or SEQ ID NO. 221
e) An AAV9-human Mpz-GJB1 vector comprising an AAV9 vector, the human myelin protein zero (hP0) promoter and an open reading frame (ORF) of the gap junction beta 1 (GJB1) gene (SEQ ID NO. 17);
f) An AAV9-human Mpz-Egfp vector comprising an AAV9 vector, the human myelin protein zero (hP0) promoter and an EGFP reporter gene (SEQ ID NO. 19);
g) An AAV9-miniMpz-SH3TC2.myc.ITR vector comprising an AAV9 vector, a minimal myelin protein zero (Mpz) promoter and an open reading frame (ORF) of the SH3TC2 gene (SEQ ID NO. 20);
h) An AAV9-human-miniMpz-SH3TC2 vector comprising an AAV9 vector, a human minimal myelin protein zero (hP0) promoter and an open reading frame (ORF) of the SH3TC2 gene (SEQ ID NO. 21);
i) An AAV9-human-miniMpz-Egfp vector comprising an AAV9 vector, a human minimal myelin protein zero (hP0) promoter and an EGFP reporter gene (SEQ ID NO. 23); or
j) an AAV9-Mpz-GJB1 vector comprising an AAV9 vector, the minimal myelin protein zero (miniMpz) promoter and an open reading frame (ORF) of the gap junction beta 1 (GJB1) gene, wherein the miniMPZ promoter has a sequence homology with at least 75% sequence homology or sequence identity with SEQ ID NO. 5 or SEQ ID NO. 22.

27. A pharmaceutical composition comprising the viral vector of claim 26, wherein the composition comprises a therapeutically appropriate amount of the viral vector and further comprises a pharmaceutically acceptable carrier and/or excipient.

28.-34. (canceled)

35. The method of claim 10, wherein the first nucleic acid sequence is a non-coding RNA, optionally wherein the non-coding RNA is a short hairpin RNA (shRNA), microRNA (miRNA), or a guide RNA (gRNA).

36. A cell that has been transduced by the viral vector of claim 26.


SEQUENCE LISTING (26 sequences)

SEQ ID NO: 1 - DNA, 6323 bp, synthetic construct
  misc_feature 1..6323: note = AAV-Mpz.Egfp construct
  source 1..6323: mol_type = other DNA
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaccgggccccccctcgaggtcgacggtatcgataagcttcctgttcagactcgtttcctgctgtaccctttcaatggccccacatcaaatcaaacacagatggcacatatctactctaaatatatgcagagcttcacaaacgtcatacacgtacgtgtgtcacacacgcacacacacacccttccacctctgcccttacctttgctgtcccatctagacattatccctcccatccccttatttcccttatcaaaatggctgctccttcaaggttccaaataacactgcttcctggacctgactcctctttcctctgaacttcctgtgttaagtgtattcctagtgcactgtgccttggtagttgttgagattgccctctgcttctcccttctgcctcctcatctagtgatcttgagcttgtagaaagaactgaattaccattctaatacgagcattctcgaactctccaaatagccaccaagcaggacaataggcagtcttgatcatttaaactgctgcatggcaaaaggaatcgaaggatttcttaacagaagtgggggggggggagatctgggcttcttcctggaagtttcctgatagagaaaatcttctgcctgggtagaatctcccaggatgcagggagatggaaaaagtgttccccaaggactttgtagtctacaggttgtggagccatcggaacaacgagacaccctaatttgggagtgctctgaaagaaacttgcctctaggccctagggctctcaggcaaggaggctaagaaggaatcctttgctgtagccttttggatttaggtttctcagcttatctatccctcagagaagtgtgtctatgtcccttttctgtccctctgcctcaccccaccccaacattccaacctagggtagggggaggtcagtatacacaaagccctctgtgtaaggggtggtatgtgtccccccacccccctacccagagtatacaatgccccttctgctccatgcccctgccaccctcccaccacctctcaattgcacatgccaggctgcaattggtcactggctcaggacagccccctcatgctggggatccaggggattttaagcaggttccagaaaacaccactcagttccttgtcccccgctctctccaccccacagacgctctgccaagcttgatatcgaattgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccctagatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 2 - DNA, 6429 bp, synthetic construct
  misc_feature 1..6429: note = AAV-Mpz.GJB1 construct
  source 1..6429: mol_type = other DNA
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtacccctcgaagcttcctgttcagactcgtttcctgctgtaccctttcaatggccccacatcaaatcaaacacagatggcacatatctactctaaatatatgcagagcttcacaaacgtcatacacgtacgtgtgtcacacacgcacacacacacccttccacctctgcccttacctttgctgtcccatctagacattatccctcccatccccttatttcccttatcaaaatggctgctccttcaaggttccaaataacactgcttcctggacctgactcctctttcctctgaacttcctgtgttaagtgtattcctagtgcactgtgccttggtagttgttgagattgccctctgcttctcccttctgcctcctcatctagtgatcttgagcttgtagaaagaactgaattaccattctaatacgagcattctcgaactctccaaatagccaccaagcaggacaataggcagtcttgatcatttaaactgctgcatggcaaaaggaatcgaaggatttcttaacagaagtgggggggggggagatctgggcttcttcctggaagtttcctgatagagaaaatcttctgcctgggtagaatctcccaggatgcagggagatggaaaaagtgttccccaaggactttgtagtctacaggttgtggagccatcggaacaacgagacaccctaatttgggagtgctctgaaagaaacttgcctctaggccctagggctctcaggcaaggaggctaagaaggaatcctttgctgtagccttttggatttaggtttctcagcttatctatccctcagagaagtgtgtctatgtcccttttctgtccctctgcctcaccccaccccaacattccaacctagggtagggggaggtcagtatacacaaagccctctgtgtaaggggtggtatgtgtccccccacccccctacccagagtatacaatgccccttctgctccatgcccctgccaccctcccaccacctctcaattgcacatgccaggctgcaattggtcactggctcaggacagccccctcatgctggggatccaggggattttaagcaggttccagaaaacaccactcagttccttgtcccccgctctctccaccccacagacgctctgccaagcttcgagaatgaggcaggatgaactggacaggtttgtacaccttgctcagtggcgtgaaccggcattctactgccattggccgagtatggctctcggtcatcttcatcttcagaatcatggtgctggtggtggctgcagagagtgtgtggggtgatgagaaatcttccttcatctgcaacacactccagcctggctgcaacagcgtttgctatgaccaattcttccccatctcccatgtgcggctgtggtccctgcagctcatcctagtttccaccccagctctcctcgtggccatgcacgtggctcaccagcaacacatagagaagaaaatgctacggcttgagggccatggggaccccctacacctggaggaggtgaagaggcacaaggtccacatctcagggacactgtggtggacctatgtcatcagcgtggtgttccggctgttgtttgaggccgtcttcatgtatgtcttttatctgctctaccctggctatgccatggtgcggctggtcaagtgcgacgtctacccctgccccaacacagtggactgcttcgtgtcccgccccaccgagaaaaccgtcttcaccgtcttcatgctagctgcctctggcatctgcatcatcctcaatgtggccgaggtggtgtacctcatcatccgggcctgtgcccgccgagcccagcgccgctccaatccaccttcccgcaagggctcgggcttcggccaccgcctctcacctgaatacaagcagaatgagatcaacaagctgctgagtgagcaggatggctccctgaaagacatactgcgccgcagccctggcaccggggctgggctggctgaaaagagcgaccgctgctcggcctgctgaggatccctcgaggtcgacggtatcgataagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 3 - DNA, 5549 bp, synthetic construct
  misc_feature 1..5549: note = AAV-miniMpz.Egfp construct
  source 1..5549: mol_type = other DNA
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaccgctctcaggcaaggaggctaagaaggaatcctttgctgtagccttttggatttaggtttctcagcttatctatccctcagagaagtgtgtctatgtcccttttctgtccctctgcctcaccccaccccaacattccaacctagggtagggggaggtcagtatacacaaagccctctgtgtaaggggtggtatgtgtccccccacccccctacccagagtatacaatgccccttctgctccatgcccctgccaccctcccaccacctctcaattgcacatgccaggctgcaattggtcactggctcaggacagccccctcatgctggggatccaggggattttaagcaggttccagaaaacaccactcagttccttgtcccccgctctctccaccccacagacgctctgccaaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccctagatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 4 - DNA, 1127 bp, Rattus sp.
  misc_feature 1..1127: note = Mpz promoter
  source 1..1127: mol_type = genomic DNA
cctgttcagactcgtttcctgctgtaccctttcaatggccccacatcaaatcaaacacagatggcacatatctactctaaatatatgcagagcttcacaaacgtcatacacgtacgtgtgtcacacacgcacacacacacccttccacctctgcccttacctttgctgtcccatctagacattatccctcccatccccttatttcccttatcaaaatggctgctccttcaaggttccaaataacactgcttcctggacctgactcctctttcctctgaacttcctgtgttaagtgtattcctagtgcactgtgccttggtagttgttgagattgccctctgcttctcccttctgcctcctcatctagtgatcttgagcttgtagaaagaactgaattaccattctaatacgagcattctcgaactctccaaatagccaccaagcaggacaataggcagtcttgatcatttaaactgctgcatggcaaaaggaatcgaaggatttcttaacagaagtgggggggggggagatctgggcttcttcctggaagtttcctgatagagaaaatcttctgcctgggtagaatctcccaggatgcagggagatggaaaaagtgttccccaaggactttgtagtctacaggttgtggagccatcggaacaacgagacaccctaatttgggagtgctctgaaagaaacttgcctctaggccctagggctctcaggcaaggaggctaagaaggaatcctttgctgtagccttttggatttaggtttctcagcttatctatccctcagagaagtgtgtctatgtcccttttctgtccctctgcctcaccccaccccaacattccaacctagggtagggggaggtcagtatacacaaagccctctgtgtaaggggtggtatgtgtccccccacccccctacccagagtatacaatgccccttctgctccatgcccctgccaccctcccaccacctctcaattgcacatgccaggctgcaattggtcactggctcaggacagccccctcatgctggggatccaggggattttaagcaggttccagaaaacaccactcagttccttgtcccccgctctctccaccccacagacgctctgcc

SEQ ID NO: 5 - DNA, 410 bp, synthetic construct
  misc_feature 1..410: note = MiniMpz promoter
  source 1..410: mol_type = other DNA
gctctcaggcaaggaggctaagaaggaatcctttgctgtagccttttggatttaggtttctcagcttatctatccctcagagaagtgtgtctatgtcccttttctgtccctctgcctcaccccaccccaacattccaacctagggtagggggaggtcagtatacacaaagccctctgtgtaaggggtggtatgtgtccccccacccccctacccagagtatacaatgccccttctgctccatgcccctgccaccctcccaccacctctcaattgcacatgccaggctgcaattggtcactggctcaggacagccccctcatgctggggatccaggggattttaagcaggttccagaaaacaccactcagttccttgtcccccgctctctccaccccacagacgctctgcc

SEQ ID NO: 6 - DNA, 852 bp, Homo sapiens
  misc_feature 1..852: note = Connexin-32 (Cx32): GenBank: AY408135.1
  source 1..852: mol_type = genomic DNA
atgaactggacaggtttgtacaccttgctcagtggcgtgaaccggcattctactgccattggccgagtatggctctcggtcatcttcatcttcagaatcatggtgctggtggtggctgcagagagtgtgtggggtgatgagaaatcttccttcatctgcaacacactccagcctggctgcaacagcgtttgctatgaccaattcttccccatctcccatgtgcggctgtggtccctgcagctcatcctagtttccaccccagctctcctcgtggccatgcacgtggctcaccagcaacacatagagaagaaaatgctacggcttgagggccatggggaccccctacacctggaggaggtgaagaggcacaaggtccacatctcagggacactgtggtggacctatgtcatcagcgtggtgttccggctgttgtttgaggccgtcttcatgtatgtcttttatctgctctaccctggctatgccatggtgcggctggtcaagtgcgacgtctacccctgccccaacacagtggactgcttcgtgtcccgccccaccgagaaaaccgtcttcaccgtcttcatgctagctgcctctggcatctgcatcatcctcaatgtggccgaggtggtgtacctcatcatccgggcctgtgcccgccgagcccagcgccgctccaatccaccttcccgcaagggctcgggcttcggccaccgcctctcacctgaatacaagcagaatgagatcaacaagctgctgagtgagcaggatggctccctgaaagacatactgcgccgcagccctggcaccggggctgggctggctgaaaagagcgaccgctgctcggcctgctga

SEQ ID NO: 7 - DNA, 3846 bp, Homo sapiens
  misc_feature 1..3846: note = SH3 domain and tetratricopeptide repeats 2 (SH3TC2): GenBank: BC114486.1
  source 1..3846: mol_type = genomic DNA
atgggtggctgcttctgcatccccagggagcggagtctgacccggggcccaggtaaagaaactccttccaaggatccaactgtatcgagtgagtgtatagcctcatctgaatacaaggaaaaatgttttctgccacagaacattaatccagacctgacactctccttctgtgtaaagagccgctccaggaggtgtgtaaatggacccctacaggaagctgctcggaggcggctctgggcactggagaatgaggaccaggaggtgcgcatgctgtttaaggacctctcagcaaggttggtcagtatccagtctcagagggcccagtttctcatcaccttcaagaccatggaggaaatctggaagttctccacctaccttaatttagaacatctcctctttgaccacaagtactggctcaactgcatattggtggaggatacagagatccaagtgtctgtagatgataaacacctggaaacaatatacctgggactcctgatacaggaaggccacttcttctgcagagccctgtgctccgtgactccaccagccgagaaggaaggggaatgcttgacactttgcaagaatgagttaatctcagtgaagatggcagaagctggctccgagttggaaggcgtgtctttggtgacaggtcagcggggcctggtactggtgtcagccttggagcctctgcctctccctttccaccagtggttcctaaagaattatccaggaagctgtggcctttccaggaagagggattggacaggctcctatcagattggcagaggacgctgtaaggccttgacgggttatgagccaggagaaaaggatgaactgaatttctaccagggagaaagcattgagatcatcggctttgtcatacctgggcttcagtggttcattggaaagtcgacaagttcaggacaagtgggctttgtccccaccaggaacatagatcctgattcttattccccaatgagcaggaactctgcctttctcagtgatgaggagagatgctccctgttggccctgggaagtgataagcagactgagtgttccagcttcctccacactcttgctcgcactgacatcacatctgtctaccggctcagtgggtttgaatccatccagaatcctccaaatgatctgagtgcatcccagcctgaaggcttcaaggaggtcaggcctggcagagcctgggaggagcatcaggccgtggggtccagacagtccagcagctctgaggactccagcctggaggaggagctcctctcggccacctcagacagctatcgcctgccggagcctgatgaccttgatgacccggaactgctcatggacctaagcactggtcaggaggaggaggctgagaacttcgcccccatattggcttttctggatcatgagggttatgctgaccactttaagagtctctatgacttctccttctctttcctcacttcttccttttatagcttctctgaggaggatgagtttgtggcctacctggaggcatcaagaaagtgggccaagaagagccacatgacctgggcccatgcccggctctgcttcctcctgggccggctgagcatcaggaaggtcaaactctctcaggccagggtgtacttcgaggaggccatccacattctcaatggagcatttgaggacctatccttggtggccactctgtacatcaatttggctgccatctacctgaaacagaggctgagacataaaggctccgccctgttggaaaaggcaggtgccctgctggcctgcctgcctgaccgtgagtctagtgccaagcatgaactcgacgtggtggcctacgtgctgcgccaggggattgtggtgggcagcagcccgctggaggccagggcctgctttctggccatccgcttgctcctgagcctaggccggcacgaggaggtcctgccctttgccgagcgcctgcagctcctctctggacaccctcctgcctctgaggctgtggccagtgttttgagttttctgtatgacaagaaatatcttccacaccttgcagtggcctctgtccagcaacatggtatccagagtgcccaagggatgtctcttcctatttggcaggtccaccttgtcctccagaacacaaccaagctccttggctttccttccccaggctggggtgaagtttctgccttggcctgcccaatgctcagacaggccctggctgcctgtgaggaactagcagaccggagcacccagagggccctgtgtctcatcctttccaaagtgtacctcgagcacaggtctcctgacggtgccatccactacctgagccaggccttggtgctagggcagctgctgggtgagcaggaatcctttgagtcttctctctgcctggcatgggcctatctcttagccagccaggccaagaaggctttggatgtgcttgagccactgctatgctccctgaaggagacagagagtctcactcaaaggggagtcatctataacctcctgggacttgcactccaaggtgaaggccgggtgaacagggcagccaagagctatcttcgggccttgaacagagcccaggaggtgggagatgtgcataaccaggcagtggctatggccaatcttggccacctgagccttaagtcctgggctcagcatccagccagaaactatctcctgcaggctgtacgactctattgtgaacttcaggccagtaaggagacagacatggaattagtacaggtgtttctctggttggcccaagttctggtgtctggacaccagctgacccatggccttctttgttatgaaatggcattgctgtttggcttaaggcatcgacatctaaagagtcagcttcaggccaccaaatccctctgccatttctacagctctgtgtccccaaaccctgaggcatgcatcacctaccatgagcactggctggccctggctcagcaactcagggaccgggagatggaagggaggctgctggagtccctggggcagctttatcggaacctaaataccgccaggtccctcaggaggtcactcacatgcatcaaggagagcctgcgtatcttcattgacctgggggagacagacaaggctgctgaggcctggcttggggcggggcgactccactacctcatgcaggaagacgagctggtggagctgtgcctgcaggcagccatccagacagccctgaagtcagaggagcctttgctggctctcaaactttatgaagaagcaggtgatgtgttcttcaatgggacccgccacaggcatcatgcagtggagtactaccgagctggagctgttcctttagcaaggaggttgaaggcggtgagaactgagctccggattttcaataagctgacagagctgcagattagcctcgaaggctatgagaaggctttggaatttgccaccctggccgccaggctcagcacagtcacaggagatcagaggcaagagctggtggcctttcaccgcctggctacagtgtactactccctgcacatgtatgagatggctgaggactgctacctgaagaccctgtccctctgtccaccctggctgcagagtcccaaggaggccctgtactatgccaaggtgtattatcgcctgggcagactcaccttctgccagctgaaggatgcccatgatgccactgagtacttccttctggccctggcagcagcggtcctgctgggtgatgaggagcttcaggacaccattaggagcaggctggacaacatctgccagagccccctgtggcacagcaggccctccgggtgctcctcagagagggcgcggtggctgagtggtggtggcctggccctctga

SEQ ID NO: 8 - DNA, 1828 bp, Homo sapiens
  misc_feature 1..1828: note = Peripheral myelin protein 22 (PMP22): NCBI Reference Sequence: NM_000304.4
  source 1..1828: mol_type = genomic DNA
agttacagggagcaccaccagggaacatctcggggagcctggttggaagctgcaggcttagtctgtcggctgcgggtctctgactgccctgtggggagggtcttgccttaacatcccttgcatttggctgcaaagaaatctgcttggaagaaggggttacgctgtttggccgggcagaaactccgctgagcagaacttgccgccagaatgctcctcctgttgctgagtatcatcgtcctccacgtcgcggtgctggtgctgctgttcgtctccacgatcgtcagccaatggatcgtgggcaatggacacgcaactgatctctggcagaactgtagcacctcttcctcaggaaatgtccaccactgtttctcatcatcaccaaacgaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgtttttgctttgcattttctgattttataccaactgtgtggactaagatgcattaaaataaacatcagagtaactca

SEQ ID NO: 9 - DNA, 810 bp, Homo sapiens
  misc_feature 1..810: note = Myelin Protein Zero (MPZ): GenBank: AK313555.1
  source 1..810: mol_type = genomic DNA
agttcctggtcccccactttctcaaccccacagatgctccgggcccctgcccctgccccagctatggctcctggggctccctcatccagccccagccctatcctggctgtgctgctcttctcttctttggtgctgtccccggcccaggccatcgtggtttacaccgacagggaggtccatggtgctgtgggctcccgggtgaccctgcactgctccttctggtccagtgagtgggtctcagatgacatctccttcacctggcgctaccagcccgaagggggcagagatgccatttcgatcttccactatgccaagggacaaccctacattgacgaggtggggaccttcaaagagcgcatccagtgggtaggggaccctcgctggaaggatggctccattgtcatacacaacctagactacagtgacaatggcacgttcacttgtgacgtcaaaaaccctccagacatagtgggcaagacctctcaggtcacgctgtatgtctttgaaaaagtgccaactaggtacggggtcgttctgggagctgtgatcgggggtgtcctcggggtggtgctgttgctgctgctgcttttctacgtggttcggtactgctggctacgcaggcaggcggccctgcagaggaggctcagtgctatggagaaggggaaattgcacaagccaggaaaggacgcgtcgaagcgcgggcggcagacgccagtgctgtatgcaatgctggaccacagcagaagcaccaaagctgtcagtgagaagaaggccaaggggctgggggagtctcgcaaggataagaaatag

SEQ ID NO: 10 - DNA, 2979 bp, Homo sapiens
  misc_feature 1..2979: note = Early Growth Response 2 (EGR2): NCBI Reference Sequence: NM_000399.5
  source 1..2979: mol_type = genomic DNA
aactgagcgaggagcaattgattaatagctcggcgaggggactcactgactgttataataacactacaccagcaactcctggcttcccagcagccggaacacagacaggagagagtcagtggcaaatagacatttttcttatttcttaaaaaacagcaacttgtttgctacttttatttctgttgatttttttttcttggtgtgtgtggtggttgtttttaagtgtggagggcaaaaggagataccatcccaggctcagtccaacccctctccaaaacggcttttctgacactccaggtagcgagggagttgggtctccaggttgtgcgaggagcaaatgatgaccgccaaggccgtagacaaaatcccagtaactctcagtggttttgtgcaccagctgtctgacaacatctacccggtggaggacctcgccgccacgtcggtgaccatctttcccaatgccgaactgggaggcccctttgaccagatgaacggagtggccggagatggcatgatcaacattgacatgactggagagaagaggtcgttggatctcccatatcccagcagctttgctcccgtctctgcacctagaaaccagaccttcacttacatgggcaagttctccattgaccctcagtaccctggtgccagctgctacccagaaggcataatcaatattgtgagtgcaggcatcttgcaaggggtcacttccccagcttcaaccacagcctcatccagcgtcacctctgcctcccccaacccactggccacaggacccctgggtgtgtgcaccatgtcccagacccagcctgacctggaccacctgtactctccgccaccgcctcctcctccttattctggctgtgcaggagacctctaccaggacccttctgcgttcctgtcagcagccaccacctccacctcttcctctctggcctacccaccacctccttcctatccatcccccaagccagccacggacccaggtctcttcccaatgatcccagactatcctggattctttccatctcagtgccagagagacctacatggtacagctggcccagaccgtaagccctttccctgcccactggacaccctgcgggtgccccctccactcactccactctctacaatccgtaactttaccctggggggccccagtgctggggtgaccggaccaggggccagtggaggcagcgagggaccccggctgcctggtagcagctcagcagcagcagcagccgccgccgccgccgcctataacccacaccacctgccactgcggcccattctgaggcctcgcaagtaccccaacagacccagcaagacgccggtgcacgagaggccctacccgtgcccagcagaaggctgcgaccggcggttctcccgctctgacgagctgacacggcacatccgaatccacactgggcataagcccttccagtgtcggatctgcatgcgcaacttcagccgcagtgaccacctcaccacccatatccgcacccacaccggtgagaagcccttcgcctgtgactactgtggccgaaagtttgcccggagtgatgagaggaagcgccacaccaagatccacctgagacagaaagagcggaaaagcagtgccccctctgcatcggtgccagccccctctacagcctcctgctctgggggcgtgcagcctgggggtaccctgtgcagcagtaacagcagcagtcttggcggagggccgctcgccccttgctcctctcggacccggacaccttgagatgagactcaggctgatacaccagctcccaaaggtcccggaggccctttgtccactggagctgcacaacaaacactaccaccctttcctgtccctctctccctttgttgggcaaagggctttggtggagctagcactgccccctttccacctagaagcaggttcttcctaaaacttagcccattctagtctctcttaggtgagttgactatcaacccaaggcaaaggggaggctcagaaggaggtggtgtggggacccctggccaagagggctgaggtctgaccctgctttaaagggttgtttgactaggttttgctaccccacttccccttattttgacccatcacaggtttttgaccctggatgtcagagttgatctaagacgttttctacaataggttgggagatgctgatcccttcaagtggggacagcaaaaagacaagcaaaactgatgtgcactttatggcttgggactgatttgggggacattgtacagtgagtgaagtatagcctttatgccacactctgtggccctaaaatggtgaatcagagcatatctagttgtctcaacccttgaagcaatatgtattataaactcagagaacagaagtgcaatgtgatgggaggaacatagcaatatctgctccttttcgagttgtttgagaaatgtaggctattttttcagtgtatatccactcagattttgtgtatttttgatgtacactgttctctaaattctgaatctttgggaaaaaatgtaaagcatttatgatctcagaggttaacttatttaagggggatgtacatatattctctgaaactaggatgcatgcaattgtgttggaagtgtccttggtgccttgtgtgatgtagacaatgttacaaggtctgcatgtaaatgggttgccttattatggagaaaaaaaatcactccctgagtttagtatggctgtatatttctgcctattaatatttggaattttttttagaaagtatatttttgtatgctttgttttgtgacttaaaagtgttacctttgtagtcaaatttcagataagaatgtacataatgttaccggagctgatttgtttggtcattagctcttaatagttgtgaaaaaataaatctattctaacgcaaaaccactaactgaagttcagataatggatggtttgtgactatagtgtaaataaatacttttcaacaata

SEQ ID NO: 11 - DNA, 1077 bp, Homo sapiens
  misc_feature 1..1077: note = Ganglioside induced differentiation associated protein 1 (GDAP1): NCBI Reference Sequence: NM_018972.3
  source 1..1077: mol_type = genomic DNA
atggctgagaggcaggaagagcagagagggagcccgcccttgagggcggaaggcaaggccgacgcggaggttaagctcattctgtaccattggacgcattccttcagctctcaaaaggtgcgcttggtaattgctgaaaaggcattgaagtgcgaggaacatgatgtaagtctgcccttgagtgagcacaatgagccttggtttatgcgtttgaactcaactggagaagtgcctgtccttatccacggggaaaacataatttgtgaggccactcagatcattgattatcttgaacagactttcctggatgaaagaacacccaggttaatgcctgataaagaaagcatgtattacccacgggtacaacattaccgagagctgcttgactccttgccaatggatgcctatacacatggctgcattttacatcctgagttaactgtggactccatgatcccggcttatgcaactacaaggattcgtagccaaattggaaacacagagtctgagctgaagaaacttgctgaagaaaacccagatttacaagaagcatacattgcaaaacagaaacgacttaaatcaaagctgcttgatcatgacaatgtcaagtatttgaagaaaattcttgatgagttggagaaagtcttggatcaggttgaaactgaattgcaaagaagaaatgaagaaaccccagaagagggccagcaaccttggctctgcggtgaatccttcaccctggcagacgtctcactcgctgtcacattgcatcgactgaagttcctggggtttgcaaggagaaactggggaaacggaaagcgaccaaacttggaaacctattacgagcgtgtcttgaagagaaaaacatttaacaaggttttaggacatgtcaacaatatattaatctctgcagtgctgccaacagcattccgggtggccaagaaaagggccccaaaagttcttggcacgacccttgtggttggtttgcttgcaggagtgggatattttgcttttatgcttttcagaaagaggcttggcagcatgatattagcatttagacccagaccaaattatttctag

SEQ ID NO: 12 - DNA, 1185 bp, Homo sapiens
  misc_feature 1..1185: note = N-Myc downstream regulated 1 (NDRG1): NCBI Reference Sequence: NM_001135242.1
  source 1..1185: mol_type = genomic DNA
atgtctcgggagatgcaggatgtagacctcgctgaggtgaagcctttggtggagaaaggggagaccatcaccggcctcctgcaagagtttgatgtccaggagcaggacatcgagactttacatggctctgttcacgtcacgctgtgtgggactcccaagggaaaccggcctgtcatcctcacctaccatgacatcggcatgaaccacaaaacctgctacaaccccctcttcaactacgaggacatgcaggagatcacccagcactttgccgtctgccacgtggacgcccctggccagcaggacggcgcagcctccttccccgcagggtacatgtacccctccatggatcagctggctgaaatgcttcctggagtccttcaacagtttgggctgaaaagcattattggcatgggaacaggagcaggcgcctacatcctaactcgatttgctctaaacaaccctgagatggtggagggccttgtccttatcaacgtgaacccttgtgcggaaggctggatggactgggccgcctccaagatctcaggatggacccaagctctgccggacatggtggtgtcccacctttttgggaaggaagaaatgcagagtaacgtggaagtggtccacacctaccgccagcacattgtgaatgacatgaaccccggcaacctgcacctgttcatcaatgcctacaacagccggcgcgacctggagattgagcgaccaatgccgggaacccacacagtcaccctgcagtgccctgctctgttggtggttggggacagctcgcctgcagtggatgccgtggtggagtgcaactcaaaattggacccaacaaagaccactctcctcaagatggcggactgtggcggcctcccgcagatctcccagccggccaagctcgctgaggccttcaagtacttcgtgcagggcatgggatacatgccctcggctagcatgacccgcctgatgcggtcccgcacagcctctggttccagcgtcacttctctggatggcacccgcagccgctcccacaccagcgagggcacccgaagccgctcccacaccagcgagggcacccgcagccgctcgcacaccagcgagggggcccacctggacatcacccccaactcgggtgctgctgggaacagcgccgggcccaagtccatggaggtctcctgctag

SEQ ID NO: 13 - DNA, 24 bp, synthetic construct
  misc_feature 1..24: note = P0-Cx32 forward primer
  source 1..24: mol_type = other DNA
aggggtacccttcctgttcagact

SEQ ID NO: 14 - DNA, 22 bp, synthetic construct
  misc_feature 1..22: note = P0-Cx32 reverse primer
  source 1..22: mol_type = other DNA
ccgctcgagggatcctcagcag

SEQ ID NO: 15 - DNA, 21 bp, synthetic construct
  misc_feature 1..21: note = KpnI-P0 forward primer
  source 1..21: mol_type = other DNA
ggggtaccgctctcaggcaag

SEQ ID NO: 16 - DNA, 23 bp, synthetic construct
  misc_feature 1..23: note = AgeI-P0 reverse primer
  source 1..23: mol_type = other DNA
aaaccggttggcagagcgtctgt

SEQ ID NO: 17 - DNA, 6324 bp, synthetic construct
  misc_feature 1..6324: note = AAV-human-Mpz-GJB1 construct
  source 1..6324: mol_type = other DNA
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaccgcctggcataaacttcatttattaaagtttattttgtctttaatctctcatataacttagtcttcctgatattgcagctgtgtgtgcccctcttttgtactcccagcattttgttcattactaaaggaagtgtcatggcttattatacttgattgttgatgggtttgtcctctgatcttcccatctccacctccccaaaccaaattttcaactccttgctggaaggacttaatttttattcctctctctattacctgcattctcatactttacatattgctggcacttaatacaattttgtagccttgaaataaattgaaatggacttaaacagcagcatgaagcactgaaggacttcttgacaaacggaaaggtcaggggcttcttgcctggaaatagtccagtggagaaaaacttctgtctgggaagaatcgcacaggatgaagggaggtgcggggaaaaaaactcccataggacttggtcatctcaagaagtctgtaatgcagcccacattagaggagataacaggggatatcctattttcagagttctctgggggaaacctccctctagttcctagggctgtgaggcagcctctctcaggcaaggaggctgaggagaaatccctttttatggcctttaaattgaggttccatatctatccctcagagaagtgtgtctgtgtccctgtttttgtccctctccctcaccaccccccacaacattccagcctggggcagggggaggccagtggacacaaagccctctgtgtatggggtggtatgtgtccccccacccctccacccagactatacaatgccccttctgctccctgcactctgcccccctccccaccacctctcaactgcacatgccaggctgcaattggttactggctgaggacagccccctcatgctggggccctaggggattttaagcaggttccaggaaccccccgttcagttcctggtcccccactttctcaaccccacagatgctccgggcccctgcccctgccccagcaccggtcgcggatcctgaggcaggatgaactggacaggtttgtacaccttgctcagtggcgtgaaccggcattctactgccattggccgagtatggctctcggtcatcttcatcttcagaatcatggtgctggtggtggctgcagagagtgtgtggggtgatgagaaatcttccttcatctgcaacacactccagcctggctgcaacagcgtttgctatgaccaattcttccccatctcccatgtgcggctgtggtccctgcagctcatcctagtttccaccccagctctcctcgtggccatgcacgtggctcaccagcaacacatagagaagaaaatgctacggcttgagggccatggggaccccctacacctggaggaggtgaagaggcacaaggtccacatctcagggacactgtggtggacctatgtcatcagcgtggtgttccggctgttgtttgaggccgtcttcatgtatgtcttttatctgctctaccctggctatgccatggtgcggctggtcaagtgcgacgtctacccctgccccaacacagtggactgcttcgtgtcccgccccaccgagaaaaccgtcttcaccgtcttcatgctagctgcctctggcatctgcatcatcctcaatgtggccgaggtggtgtacctcatcatccgggcctgtgcccgccgagcccagcgccgctccaatccaccttcccgcaagggctcgggcttcggccaccgcctctcacctgaatacaagcagaatgagatcaacaagctgctgagtgagcaggatggctccctgaaagacatactgcgccgcagccctggcaccggggctgggctggctgaaaagagcgaccgctgctcggcctgctgactcgagatcgatatccatcacactggcggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 18 - DNA, 1024 bp, Homo sapiens
  misc_feature 1..1024: note = Human hP0 promoter
  source 1..1024: mol_type = genomic DNA
gcctggcataaacttcatttattaaagtttattttgtctttaatctctcatataacttagtcttcctgatattgcagctgtgtgtgcccctcttttgtactcccagcattttgttcattactaaaggaagtgtcatggcttattatacttgattgttgatgggtttgtcctctgatcttcccatctccacctccccaaaccaaattttcaactccttgctggaaggacttaatttttattcctctctctattacctgcattctcatactttacatattgctggcacttaatacaattttgtagccttgaaataaattgaaatggacttaaacagcagcatgaagcactgaaggacttcttgacaaacggaaaggtcaggggcttcttgcctggaaatagtccagtggagaaaaacttctgtctgggaagaatcgcacaggatgaagggaggtgcggggaaaaaaactcccataggacttggtcatctcaagaagtctgtaatgcagcccacattagaggagataacaggggatatcctattttcagagttctctgggggaaacctccctctagttcctagggctgtgaggcagcctctctcaggcaaggaggctgaggagaaatccctttttatggcctttaaattgaggttccatatctatccctcagagaagtgtgtctgtgtccctgtttttgtccctctccctcaccaccccccacaacattccagcctggggcagggggaggccagtggacacaaagccctctgtgtatggggtggtatgtgtccccccacccctccacccagactatacaatgccccttctgctccctgcactctgcccccctccccaccacctctcaactgcacatgccaggctgcaattggttactggctgaggacagccccctcatgctggggccctaggggattttaagcaggttccaggaaccccccgttcagttcctggtcccccactttctcaaccccacagatgctccgggcccctgcccctgccccagc

SEQ ID NO: 19 - DNA, 6162 bp, synthetic construct
  misc_feature 1..6162: note = AAV-human-Mpz-Egfp mock construct
  source 1..6162: mol_type = other DNA
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaccgcctggcataaacttcatttattaaagtttattttgtctttaatctctcatataacttagtcttcctgatattgcagctgtgtgtgcccctcttttgtactcccagcattttgttcattactaaaggaagtgtcatggcttattatacttgattgttgatgggtttgtcctctgatcttcccatctccacctccccaaaccaaattttcaactccttgctggaaggacttaatttttattcctctctctattacctgcattctcatactttacatattgctggcacttaatacaattttgtagccttgaaataaattgaaatggacttaaacagcagcatgaagcactgaaggacttcttgacaaacggaaaggtcaggggcttcttgcctggaaatagtccagtggagaaaaacttctgtctgggaagaatcgcacaggatgaagggaggtgcggggaaaaaaactcccataggacttggtcatctcaagaagtctgtaatgcagcccacattagaggagataacaggggatatcctattttcagagttctctgggggaaacctccctctagttcctagggctgtgaggcagcctctctcaggcaaggaggctgaggagaaatccctttttatggcctttaaattgaggttccatatctatccctcagagaagtgtgtctgtgtccctgtttttgtccctctccctcaccaccccccacaacattccagcctggggcagggggaggccagtggacacaaagccctctgtgtatggggtggtatgtgtccccccacccctccacccagactatacaatgccccttctgctccctgcactctgcccccctccccaccacctctcaactgcacatgccaggctgcaattggttactggctgaggacagccccctcatgctggggccctaggggattttaagcaggttccaggaaccccccgttcagttcctggtcccccactttctcaaccccacagatgctccgggcccctgcccctgccccagcaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccctagatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 20 - DNA, 7916 bp, synthetic construct
  misc_feature 1..7916: note = AAV-minMpz-SH3TC2.myc.ITR
  source 1..7916: mol_type = other DNA
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaccgctctcaggcaaggaggctaagaaggaatcctttgctgtagccttttggatttaggtttctcagcttatctatccctcagagaagtgtgtctatgtcccttttctgtccctctgcctcaccccaccccaacattccaacctagggtagggggaggtcagtatacacaaagccctctgtgtaaggggtggtatgtgtccccccacccccctacccagagtatacaatgccccttctgctccatgcccctgccacccctcccaccacctctcaattgcacatgccaggctgcaattggtcactggctcaggacagccccctcatgctggggatccaggggattttaagcaggttccagaaaacaccactcagttccttgtcccccgctctctccaccccacagacgctctgccaaccggtaccatgggtggctgcttctgcatccccagggagcggagtctgacccggggcccaggtaaagaaactccttccaaggatccaactgtatcgagtgagtgtatagcctcatctgaatacaaggaaaaatgttttctgccacagaacattaatccagacctgacactctccttctgtgtaaagagccgctccaggaggtgtgtaaatggacccctacaggaagctgctcggaggcggctctgggcactggagaatgaggaccaggaggtgcgcatgctgtttaaggacctctcagcaaggttggtcagtatccagtctcagagggcccagtttctcatcaccttcaagaccatggaggaaatctggaagttctccacctaccttaatttaggctacgtatccatgtgtctagaacatctcctctttgaccacaagtactggctcaactgcatattggtggaggatacagagatccaagtgtctgtagatgataaacacctggaaacaatatacctgggactcctgatacaggaaggccacttcttctgcagagccctgtgctccgtgactccaccagccgagaaggaaggggaatgcttgacactttgcaagaatgagttaatctcagtgaagatggcagaagctggctccgagttggaaggcgtgtctttggtgacaggtcagcggggcctggtactggtgtcagccttggagcctctgcctctccctttccaccagtggttcctaaagaattatccaggaagctgtggcctttccaggaagagggattggacaggctcctatcagattggcagaggacgctgtaaggccttgacgggttatgagccaggagaaaaggatgaactgaatttctaccagggagaaagcattgagatcatcggctttgtcatacctgggcttcagtggttcattggaaagtcgacaagttcaggacaagtgggctttgtccccaccaggaacatagatcctgattcttattccccaatgagcaggaactctgcctttctcagtgatgaggagagatgctccctgttggccctgggaagtgataagcagactgagtgttccagcttcctccacactcttgctcgcactgacatcacatctgtctaccggctcagtgggtttgaatccatccagaatcctccaaatgatctgagtgcatcccagcctgaaggtttcaaggaggtcaggcctggcagagcctgggaggagcatcaggccgtggggtccagacagtccagcagctctgaggactccagcctggaggaggagctcctctcggccacctcagacagctatcgcctgccggagcctgatgaccttgatgacccggaactgctcatggacctaagcactggtcaggaggaggaggctgagaacttcgcccccatattggcttttctggatcatgagggttatgctgaccactttaagagtctctatgacttctccttctctttcctcacttcttccttttatagcttctctgaggaggatgagtttgtggcctacctggaggcatcaagaaagtgggccaagaagagccacatgacctgggcccatgcccggctctgcttcctcctgggccggctgagcatcaggaaggtcaaactctctcaggccagggtgtacttcgaggaggccatccacattctcaatggagcatttgaggacctatccttggtggccactctgtacatcaatttggctgccatctacctgaaacagaggctgagacataaaggctccgccctgttggaaaaggcaggtgccctgctggcctgcctgcctgaccgtgagtctagtgccaagcatgaactcgacgtggtggcctacgtgctgcgccaggggattgtggtgggcagcagcccgctggaggccagggcctgctttctggccatccgcttgctcctgagcctaggccggcacgaggaggtcctgccctttgccgagcgcctgcagctcctctctggacaccctcctgcctctgaggctgtggccagtgttttgagttttctgtatgacaagaaatatcttccacaccttgcagtggcctctgtccagcaacatggtatccagagtgcccaagggatgtctcttcctatttggcaggtccaccttgtcctccagaacacaaccaagctccttggctttccttccccaggctggggtgaagtttctgccttggcctgcccaatgctcagacaggccctggctgcctgtgaggaactagcagaccggagcacccagagggccctgtgtctcatcctttccaaagtgtacctcgagcacaggtctcctgacggtgccatccactacctgagccaggccttggtgctagggcagctgctgggtgagcaggaatcctttgagtcttctctctgcctggcatgggcctatctcttagccagccaggccaagaaggctttggatgtgcttgagccactgctatgctccctgaaggagacagagagtctcactcaaaggggagtcatctataacctcctgggacttgcactccaaggtgaaggccgggtgaacagggcagccaagagctatcttcgggccttgaacagagcccaggaggtgggagatgtgcataaccaggcagtggctatggccaatcttggccacctgagccttaagtcctgggctcagcatccagccagaaactatctcctgcaggctgtacgactctattgtgaacttcaggccagtaaggagacagacatggaattagtacaggtgtttctctggttggcccaagttctggtgtctggacaccagctgacccatggccttctttgttatgaaatggcattgctgtttggcttaaggcatcgacatctaaagagtcagcttcaggccaccaaatccctctgccatttctacagctctgtgtccccaaaccctgaggcatgcatcacctaccatgagcactggctggccctggctcagcaactcagggaccgggagatggaagggaggctgctggagtccctggggcagctttatcggaacctaaataccgccaggtccctcaggaggtcactcacatgcatcaaggagagcctgcgtatcttcattgacctgggggagacagacaaggctgctgaggcctggcttggggcggggcgactccactacctcatgcaggaagacgagctggtggagctgtgcctgcaggcagccatccagacagccctgaagtcagaggagcctttgctggctctcaaactttatgaagaagcaggtgatgtgttcttcaatgggacccgccacaggcatcatgcagtggagtactaccgagctggagctgttcctttagcaaggaggttgaaggcggtgagaactgagctccggattttcaataagctgacagagctgcagattagcctcgaaggctatgagaaggctttggaatttgccaccctggccgccaggctcagcacagtcacaggagatcagaggcaagagctggtggcctttcaccgcctggctacagtgtactactccctgcacatgtatgagatggctgaggactgctacctgaagaccctgtccctctgtccaccatggctgcagagtcccaaggaggccctgtactatgccaaggtgtattatcgcctgggcagactcaccttctgccagctgaaggatgcccatgatgccactgagtacttccttctggccctggcagcagcggtcctgctgggtgatgaggagcttcaggacaccattaggagcaggctggacaacatctgccagagccccctgtggcacagcaggccctccgggtgctcctcagagagggcgcggtggctgagtggtggtggcctggccctcgagcagaagctgatcagcgaggaggacctgtaagatatccatcacactggcggccgcggagctctcgagaggcctaataaagagctcagatgcatcgatcagagtgtgttggttttttgtgtgagatctaagcttagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 21 - DNA, 7884 bp, synthetic construct
  misc_feature 1..7884: note = AAV-human-miniMpz-SH3TC2
  source 1..7884: mol_type = other DNA
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtacctctctcaggcaaggaggctgaggagaaatccctttttatggcctttaaattgaggttccatatctatccctcagagaagtgtgtctgtgtccctgtttttgtccctctccctcaccaccccccacaacattccagcctggggcagggggaggccagtggacacaaagccctctgtgtatggggtggtatgtgtccccccacccctccacccagactatacaatgccccttctgctccctgcactctgcccccctccccaccacctctcaactgcacatgccaggctgcaattggttactggctgaggacagccccctcatgctggggccctaggggattttaagcaggttccaggaaccccccgttcagttcctggtcccccactttctcaaccccacagatgctccgggcccctgcccctgccccagcggtaccatgggtggctgcttctgcatccccagggagcggagtctgacccggggcccaggtaaagaaactccttccaaggatccaactgtatcgagtgagtgtatagcctcatctgaatacaaggaaaaatgttttctgccacagaacattaatccagacctgacactctccttctgtgtaaagagccgctccaggaggtgtgtaaatggacccctacaggaagctgctcggaggcggctctgggcactggagaatgaggaccaggaggtgcgcatgctgtttaaggacctctcagcaaggttggtcagtatccagtctcagagggcccagtttctcatcaccttcaagaccatggaggaaatctggaagttctccacctaccttaatttaggctacgtatccatgtgtctagaacatctcctctttgaccacaagtactggctcaactgcatattggtggaggatacagagatccaagtgtctgtagatgataaacacctggaaacaatatacctgggactcctgatacaggaaggccacttcttctgcagagccctgtgctccgtgactccaccagccgagaaggaaggggaatgcttgacactttgcaagaatgagttaatctcagtgaagatggcagaagctggctccgagttggaaggcgtgtctttggtgacaggtcagcggggcctggtactggtgtcagccttggagcctctgcctctccctttccaccagtggttcctaaagaattatccaggaagctgtggcctttccaggaagagggattggacaggctcctatcagattggcagaggacgctgtaaggccttgacgggttatgagccaggagaaaaggatgaactgaatttctaccagggagaaagcattgagatcatcggctttgtcatacctgggcttcagtggttcattggaaagtcgacaagttcaggacaagtgggctttgtccccaccaggaacatagatcctgattcttattccccaatgagcaggaactctgcctttctcagtgatgaggagagatgctccctgttggccctgggaagtgataagcagactgagtgttccagcttcctccacactcttgctcgcactgacatcacatctgtctaccggctcagtgggtttgaatccatccagaatcctccaaatgatctgagtgcatcccagcctgaaggtttcaaggaggtcaggcctggcagagcctgggaggagcatcaggccgtggggtccagacagtccagcagctctgaggactccagcctggaggaggagctcctctcggccacctcagacagctatcgcctgccggagcctgatgaccttgatgacccggaactgctcatggacctaagcactggtcaggaggaggaggctgagaacttcgcccccatattggcttttctggatcatgagggttatgctgaccactttaagagtctctatgacttctccttctctttcctcacttcttccttttatagcttctctgaggaggatgagtttgtggcctacctggaggcatcaagaaagtgggccaagaagagccacatgacctgggcccatgcccggctctgcttcctcctgggccggctgagcatcaggaaggtcaaactctctcaggccagggtgtacttcgaggaggccatccacattctcaatggagcatttgaggacctatccttggtggccactctgtacatcaatttggctgccatctacctgaaacagaggctgagacataaaggctccgccctgttggaaaaggcaggtgccctgctggcctgcctgcctgaccgtgagtctagtgccaagcatgaactcgacgtggtggcctacgtgctgcgccaggggattgtggtgggcagcagcccgctggaggccagggcctgctttctggccatccgcttgctcctgagcctaggccggcacgaggaggtcctgccctttgccgagcgcctgcagctcctctctggacaccctcctgcctctgaggctgtggccagtgttttgagttttctgtatgacaagaaatatcttccacaccttgcagtggcctctgtccagcaacatggtatccagagtgcccaagggatgtctcttcctatttggcaggtccaccttgtcctccagaacacaaccaagctccttggctttccttccccaggctggggtgaagtttctgccttggcctgcccaatgctcagacaggccctggctgcctgtgaggaactagcagaccggagcacccagagggccctgtgtctcatcctttccaaagtgtacctcgagcacaggtctcctgacggtgccatccactacctgagccaggccttggtgctagggcagctgctgggtgagcaggaatcctttgagtcttctctctgcctggcatgggcctatctcttagccagccaggccaagaaggctttggatgtgcttgagccactgctatgctccctgaaggagacagagagtctcactcaaaggggagtcatctataacctcctgggacttgcactccaaggtgaaggccgggtgaacagggcagccaagagctatcttcgggccttgaacagagcccaggaggtgggagatgtgcataaccaggcagtggctatggccaatcttggccacctgagccttaagtcctgggctcagcatccagccagaaactatctcctgcaggctgtacgactctattgtgaacttcaggccagtaaggagacagacatggaattagtacaggtgtttctctggttggcccaagttctggtgtctggacaccagctgacccatggccttctttgttatgaaatggcattgctgtttggcttaaggcatcgacatctaaagagtcagcttcaggccaccaaatccctctgccatttctacagctctgtgtccccaaaccctgaggcatgcatcacctaccatgagcactggctggccctggctcagcaactcagggaccgggagatggaagggaggctgctggagtccctggggcagctttatcggaacctaaataccgccaggtccctcaggaggtcactcacatgcatcaaggagagcctgcgtatcttcattgacctgggggagacagacaaggctgctgaggcctggcttggggcggggcgactccactacctcatgcaggaagacgagctggtggagctgtgcctgcaggcagccatccagacagccctgaagtcagaggagcctttgctggctctcaaactttatgaagaagcaggtgatgtgttcttcaatgggacccgccacaggcatcatgcagtggagtactaccgagctggagctgttcctttagcaaggaggttgaaggcggtgagaactgagctccggattttcaataagctgacagagctgcagattagcctcgaaggctatgagaaggctttggaatttgccaccctggccgccaggctcagcacagtcacaggagatcagaggcaagagctggtggcctttcaccgcctggctacagtgtactactccctgcacatgtatgagatggctgaggactgctacctgaagaccctgtccctctgtccaccatggctgcagagtcccaaggaggccctgtactatgccaaggtgtattatcgcctgggcagactcaccttctgccagctgaaggatgcccatgatgccactgagtacttccttctggccctggcagcagcggtcctgctgggtgatgaggagcttcaggacaccattaggagcaggctggacaacatctgccagagccccctgtggcacagcaggccctccgggtgctcctcagagagggcgcggtggctgagtggtggtggcctggccctctgagcggccgcggagctctcgagaggcctaataaagagctcagatgcatcgatcagagtgtgttggttttttgtgtgagatctaagcttagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 22 - DNA, 429 bp, synthetic construct
  misc_feature 1..429: note = Mini-human hP0 promoter
  source 1..429: mol_type = other DNA
tctctcaggcaaggaggctgaggagaaatccctttttatggcctttaaattgaggttccatatctatccctcagagaagtgtgtctgtgtccctgtttttgtccctctccctcaccaccccccacaacattccagcctggggcagggggaggccagtggacacaaagccctctgtgtatggggtggtatgtgtccccccacccctccacccagactatacaatgccccttctgctccctgcactctgcccccctccccaccacctctcaactgcacatgccaggctgcaattggttactggctgaggacagccccctcatgctggggccctaggggattttaagcaggttccaggaaccccccgttcagttcctggtcccccactttctcaaccccacagatgctccgggcccctgcccctgccccagc

SEQ ID NO: 23 - DNA, 5567 bp, synthetic construct
  misc_feature 1..5567: note = AAV-human-miniMpz-Egfp
  source 1..5567: mol_type = other DNA
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtacctctctcaggcaaggaggctgaggagaaatccctttttatggcctttaaattgaggttccatatctatccctcagagaagtgtgtctgtgtccctgtttttgtccctctccctcaccaccccccacaacattccagcctggggcagggggaggccagtggacacaaagccctctgtgtatggggtggtatgtgtccccccacccctccacccagactatacaatgccccttctgctccctgcactctgcccccctccccaccacctctcaactgcacatgccaggctgcaattggttactggctgaggacagccccctcatgctggggccctaggggattttaagcaggttccaggaaccccccgttcagttcctggtcccccactttctcaaccccacagatgctccgggcccctgcccctgccccagcaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccctagatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 24 - DNA, 72 bp, synthetic construct
  misc_feature 1..72: note = Minimal synthetic poly A sequence
  source 1..72: mol_type = other DNA
ggagctctcgagaggcctaataaagagctcagatgcatcgatcagagtgtgttggttttttgtgtgagatct

SEQ ID NO: 25 - DNA, 22 bp, synthetic construct
  misc_feature 1..22: note = KpnhP0 forward primer
  source 1..22: mol_type = other DNA
aggggtaccgcctggcataaac

SEQ ID NO: 26 - DNA, 21 bp, synthetic construct
  misc_feature 1..21: note = AgehP0 reverse primer
  source 1..21: mol_type = other DNA
aatttaccggtgctggggcag